(19) United States
(12) Patent Application Publication
(10) Pub. No.: US 2010/0120784 A1
(43) Pub. Date: May 13, 2010

(54) NOVEL HETEROAROMATIC COMPOUNDS AS INHIBITORS OF STEAROYL-COENZYME A DELTA-9 DESATURASE

(76) Inventors: Nicolas Lachance, Pierrefonds (CA); Chun Sing Li, Dollard-des-Ormeaux (CA); Jean-Philppe Leclerc, Laval (CA); Yeeman K. Ramtohul, Pierrefonds (CA)

Correspondence Address: MERCK, P O BOX 2000, RAHWAY, NJ, 07065-0907, US

(21) Appl. No.: 12/594,615

(22) PCT Filed: Apr. 17, 2008
(86) PCT No.: PCT/CA08/00721

§ 371 Date: Oct. 5, 2009

Related U.S. Application Data
(60) Provisional application No. 60/925,530, filed on Apr. 20, 2007.

(51) Int. Cl. A61K 31/501; C07D 261/12; C07D 417/04; C07D 401/04; C07D 403/04; C07D 257/04; C07D 277/14; A61K 31/42; A61K 31/433; A61K 31/496; A61K 31/497; A61K 31/41; A61K 31/427; A61P 9/10; A61P 3/10; A61P 25/00; A61P 35/00

(52) U.S. Cl. 514/252.05; 548/243; 548/137; 544/333; 544/405; 544/238; 548/254; 548/187; 514/380; 514/363; 514/256; 514/255.05; 514/381; 514/369

(57) ABSTRACT

Heteroaromatic compounds of structural formula (I) or a pharmaceutically acceptable salt thereof, wherein W is a substituted heteroaryl, X and Y are each independently a bond, —O—, —S—, —S(O)—, —S(O)₂—, —NR⁶—, —C(O)—, —C(CH₃)(OH)— or —C(CH₃)═CH—, u (I) is an integer from 1 to 4, and Ar is an optionally substituted phenyl or naphtyl, are inhibitors of stearoyl-coenzyme A delta-9 desaturase (SCD) The compounds of the present invention are useful for the prevention and treatment of conditions related to abnormal lipid synthesis and metabolism, including cardiovascular disease, such as atherosclerosis, obesity, Type 2 diabetes, insulin resistance, hyperglycemia, Metabolic Syndrome, neurological disease, cancer, and liver steatosis

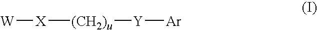

### FIELD OF THE INVENTION

The present invention relates to novel heteroaromatic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease, such as atherosclerosis; obesity; diabetes; neurological disease; metabolic syndrome; insulin resistance; cancer; and hepatic steatosis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCD's) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2 or SCD5)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCD's in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD gene (Zheng et al., Nature Genetics, 23: 268-270 (1999)), b) SCD-null mice from targeted gene deletion (Ntambi, et al., PNAS, 99: 11482-11486 (2002), and c) the suppression of SCD expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. SCD knock-out mice (−/−) are characterized by reduced adiposity and increased energy expenditure. Thus, SCD inhibition represents a novel therapeutic strategy in the treatment of Type 2 diabetes, obesity, and related metabolic disorders, such as the Metabolic Syndrome.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). The postprandial de novo lipogenesis is significantly elevated in obese subjects (Marques-Lopes, et al., American Journal of Clinical Nutrition, 73: 252-261 (2001)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications: WO 2005/011653; WO 2005/011654; WO 2005/011656; WO 2005/011657; WO 2006/014168; WO 2006/034279; WO 2006/034312; WO 2006/034315; WO 2006/034338; WO 2006/034341; WO 2006/034440; WO 2006/034441; WO 2006/034446; WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; and WO 2007/136746, all assigned to Xenon Pharmaceuticals, Inc. A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/038865 (12 Apr. 2007); WO 2007/056846 (24 May 2007); WO 2007/071023 (28 Jun. 2007); WO 2007/134457 (29 Nov. 2007); WO 2007/143823 (21 Dec. 2007); and WO 2007/143824 (21 Dec. 2007). WO 2008/003753 (assigned to Novartis) discloses a series of pyrazolo[1,5-a]pyrimidine analogs as SCD inhibitors, and WO 2007/143597 (assigned to Novartis and Xenon Pharmaceuticals) discloses heterocyclic derivatives as SCD inhibitors. Small molecule SCD inhibitors have also been described by G. Liu, et al., “Discovery of Potent, Selective, Orally Bioavailable SCD1 Inhibitors,” in J. Med. Chem., 50: 3086-3100 (2007) and by H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, hyperglycemia, Type 2 diabetes, Metabolic Syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobryzn and J. M. Ntambi, in “Stearoyl-CoA desaturase as a new drug target for obesity treatment,” Obesity Reviews, 6: 169-174 (2005).

### SUMMARY OF THE INVENTION

The present invention relates to heteroaromatic compounds of structural formula I:

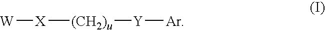

These heteroaromatic compounds are effective as inhibitors of SCD. They are therefore useful for the treatment, control or prevention of disorders responsive to the inhibition of SCD, such as Type 2 diabetes, insulin resistance, hyperglycemia, lipid disorders, obesity, atherosclerosis, and Metabolic Syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, hyperglycemia, insulin resistance, obesity, lipid disorders, atherosclerosis, and Metabolic Syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with novel heteroaromatic compounds useful as inhibitors of SCD. Compounds of the present invention are described by structural formula I:

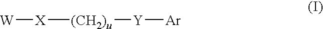

or a pharmaceutically acceptable salt thereof; wherein any methylene (CH₂) carbon atom in (CH₂)ᵤ is optionally substituted with one to two R⁵ substituents independently selected from fluorine, hydroxy, oxo, hydroxymethyl, and C₁₋₄ alkyl; or two R⁵ substituents, when on the same (CH₂) carbon atom, are taken together with the carbon atom to which they are attached to form a C₃₋₆ cycloalkyl group; or any two methylene (CH₂) carbon atoms are taken together to form a saturated or monounsaturated five- or six-membered cycloalkyl group;
X and Y are each independently a bond, —O—, —S—, —S(O)—, —S(O)₂—, —NR⁶—,

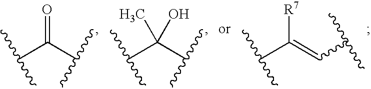

W is heteroaryl selected from the group consisting of:

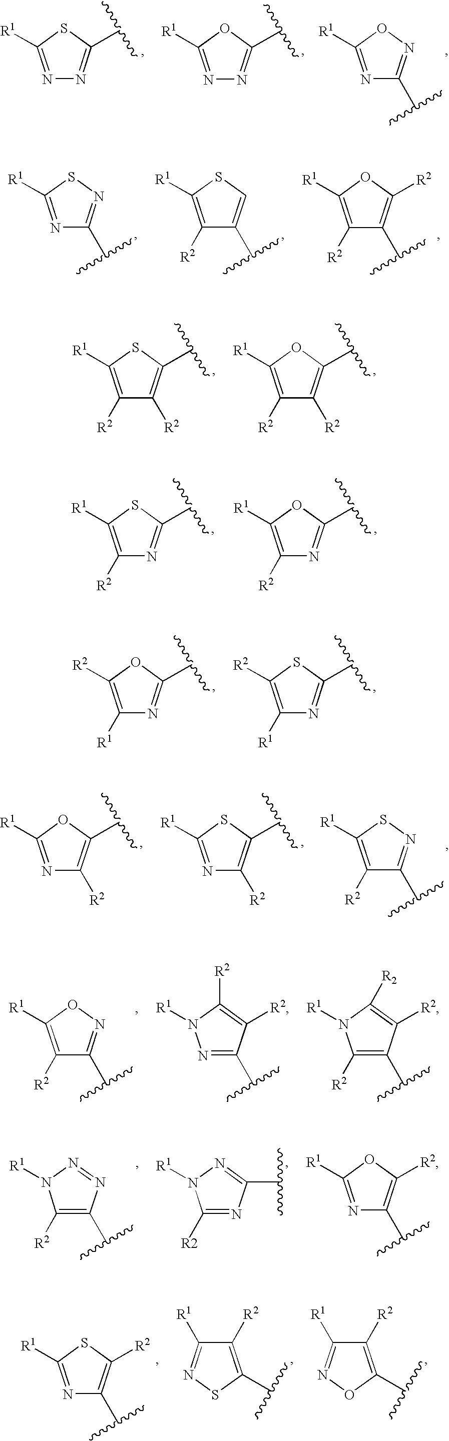

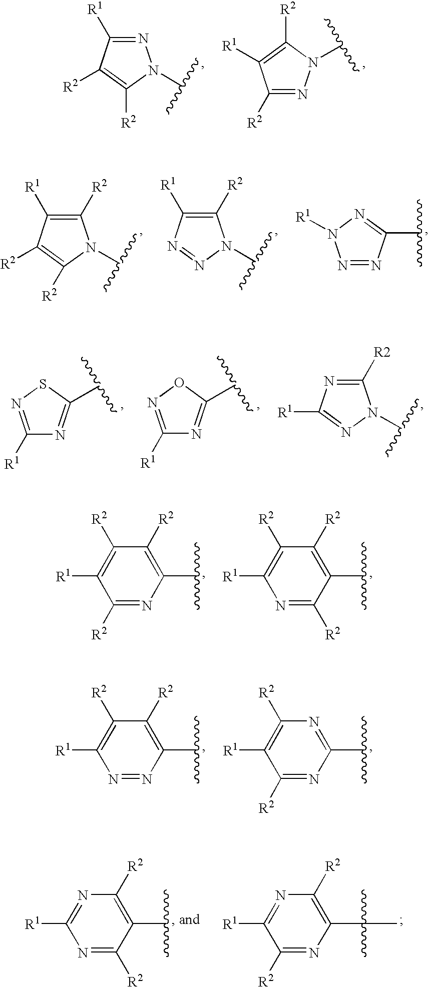

R¹ is heteroaryl selected from the group consisting of:

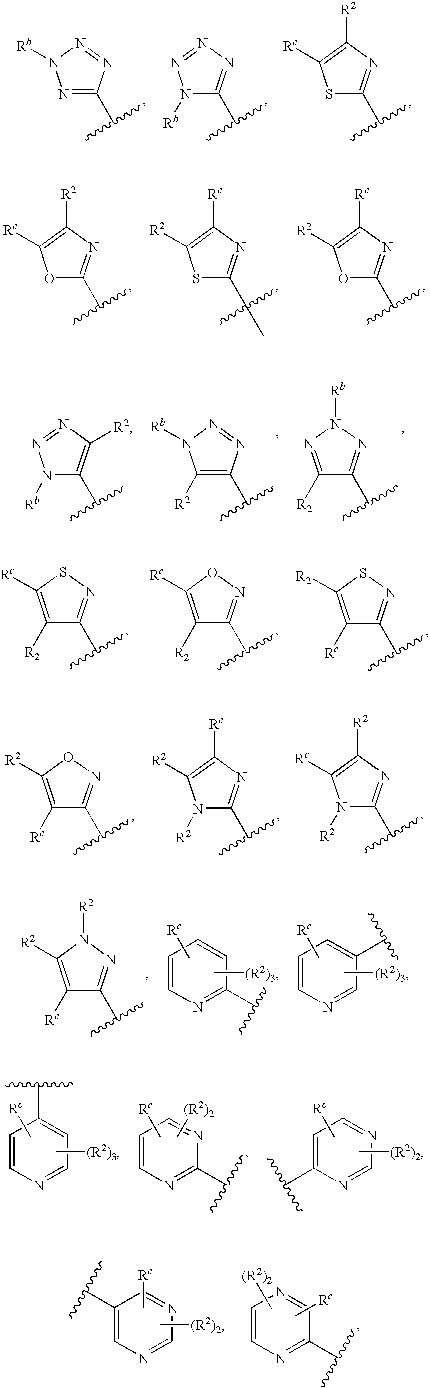

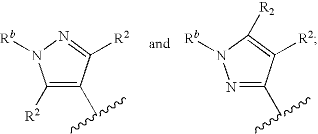

wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
and wherein said R¹ heteroaryl ring is optionally substituted with one substituent independently selected from the group consisting of cyano, halogen, C₁₋₄ alkyl, C₁₋₄ alkoxy, C₁₋₄ alkylthio, C₁₋₄ alkylsulfonyl, and trifluoromethyl;

each R² is independently selected from the group consisting of:

hydrogen,

halogen,

hydroxy,

cyano,

amino,

nitro,

C₁₋₄ alkyl, optionally substituted with one to five fluorines,

C₁₋₄ alkoxy, optionally substituted with one to five fluorines,

C₁₋₄ alkylthio, optionally substituted with one to five fluorines,

C₁₋₄ alkylsulfonyl,

carboxy,

C₁₋₄ alkyloxycarbonyl, and

C₁₋₄ alkylcarbonyl;

Ar is phenyl or naphthyl optionally substituted with one to five R³ substituents;
each R³ is independently selected from the group consisting of:

C₁₋₆ alkyl,

C₂₋₆ alkenyl,

(CH₂)ₙ-phenyl,

(CH₂)ₙ-naphthyl,

(CH₂)ₙ-heteroaryl,

(CH₂)ₙ-heterocyclyl,

(CH₂)ₙC₃₋₇ cycloalkyl,

halogen,

nitro,

(CH₂)ₙOR⁴,

(CH₂)ₙN(R⁴)₂,

(CH₂)ₙC≡N,

(CH₂)ₙCO₂R⁴,

(CH₂)ₙNR⁴SO₂R⁴

(CH₂)ₙSO₂N(R⁴)₂,

(CH₂)ₙS(O)₀₋₂R⁴,

(CH₂)ₙNR⁴C(O)N(R⁴)₂,

(CH₂)ₙC(O)N(R⁴)₂,

(CH₂)ₙNR⁴C(O)R⁴,

(CH₂)ₙNR⁴CO₂R⁴,

(CH₂)ₙC(O)R⁴,

O(CH₂)ₙC(O)N(R⁴)₂,

(CH₂)ₛ—Z—(CH₂)ₜ-phenyl,

(CH₂)ₛ—Z—(CH₂)ₜ-naphthyl,

(CH₂)ₛ—Z—(CH₂)ₜ-heteroaryl,

(CH₂)ₛ—Z—(CH₂)ₜ-heterocyclyl,

(CH₂)ₛ—Z—(CH₂)ₜ—C₃₋₇ cycloalkyl,

(CH₂)ₛ—Z—(CH₂)ₜ—OR⁴,

(CH₂)ₛ—Z—(CH₂)ₜ—N(R⁴)₂,

(CH₂)ₛ—Z—(CH₂)ₜ—NR⁴SO₂R⁴,

(CH₂)ₛ—Z—(CH₂)ₜ—C≡N,

(CH₂)ₛ—Z—(CH₂)ₜ—CO₂R⁴,

(CH₂)ₛ—Z—(CH₂)ₜ—SO₂N(R⁴)₂,

(CH₂)ₛ—Z—(CH₂)ₜ—S(O)₀₋₂R⁴,

(CH₂)ₛ—Z—(CH₂)ₜ—NR⁴C(O)N(R⁴)₂,

(CH₂)ₛ—Z—(CH₂)ₜ—C(O)N(R⁴)₂,

(CH₂)ₛ—Z—(CH₂)ₜ—NR⁴C(O)R⁴,

(CH₂)ₛ—Z—(CH₂)ₜ—NR⁴CO₂R⁴,

(CH₂)ₛ—Z—(CH₂)ₜ—C(O)R⁴,

CF₃,

CH₂CF₃,

OCF₃, and

OCH₂CF₃;

in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy optionally substituted with one to five fluorines; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
each R⁴ is independently selected from the group consisting of

hydrogen,

C₁₋₆ alkyl,

(CH₂)ₙ-phenyl,

(CH₂)ₙ-heteroaryl,

(CH₂)ₙ-naphthyl, and

(CH₂)ₙC₃₋₇ cycloalkyl;

wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
each R⁶ and R⁷ are independently hydrogen or C₁₋₃ alkyl, wherein alkyl is optionally substituted with one to five fluorines;
u is an integer from 1 to 4;
r is an integer from 1 to 3;
m is an integer from 0 to 3;
each p is independently an integer from 1 to 3;
each n is independently an integer from 0 to 2;
each s is independently an integer from 1 to 3; and
each t is independently an integer from 1 to 3.

In one embodiment of the compounds of the present invention, X and Y are both O.

In a second embodiment of the compounds of the present invention, u is 3. In a class of this embodiment, X and Y are both O. In another class of this embodiment, X is S and Y is O.

In a third embodiment, compounds of the present invention are of structural formula (II):

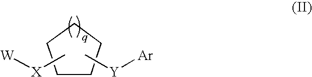

wherein q is 1 or 2, and W, X, Y, and Ar are as defined above. In a class of this embodiment, compounds of the present invention are of structural formula (II):

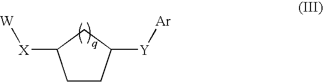

wherein q, W, X, Y, and Ar are as defined above. In a subclass of this class, q is 2, and X and Y are both O.

In a fourth embodiment of the compounds of the present invention, Ar is phenyl substituted with one to three R³ substituents as defined above.

In a fifth embodiment of the compounds of the present invention, W is heteroaryl selected from the group consisting of:

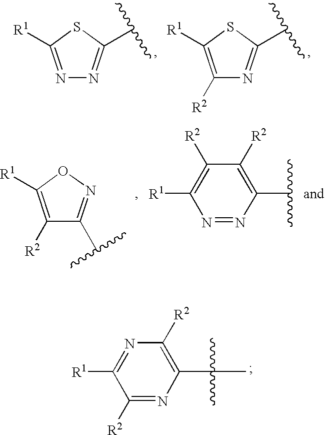

wherein R¹ and R² are as defined above. In a class of this embodiment, R² is hydrogen. In another class of this embodiment, W is

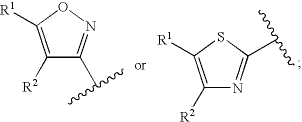

wherein R¹ and R² are as defined above. In a subclass of this class, R² is hydrogen. In another subclass of this class, W is

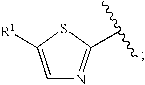

wherein R¹ is as defined above.

In a sixth embodiment of the compounds of the present invention, R¹ is heteroaryl selected from the group consisting of

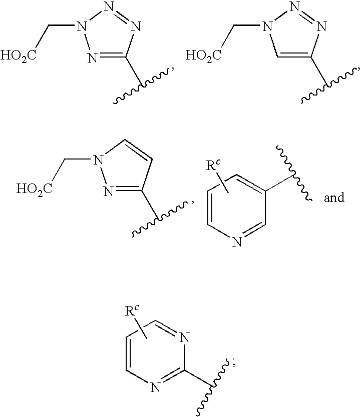

wherein R^{c} is —CO₂H, —CO₂C₁₋₃ alkyl, —CH₂CO₂H, or —CH₂CO₂C₁₋₃ alkyl. In a class of this embodiment, R¹ is

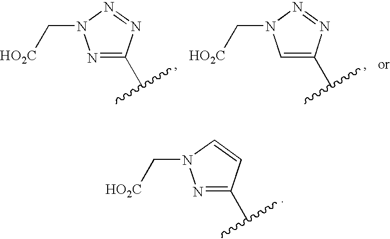

In a seventh embodiment of the compounds of the present invention, W is heteroaryl selected from the group consisting of:

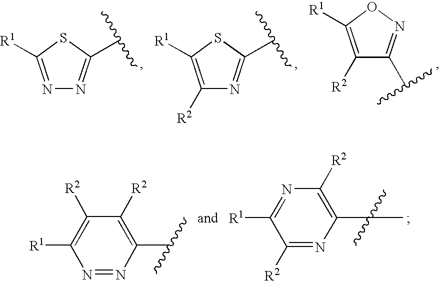

and R¹ is heteroaryl selected from the group consisting of:

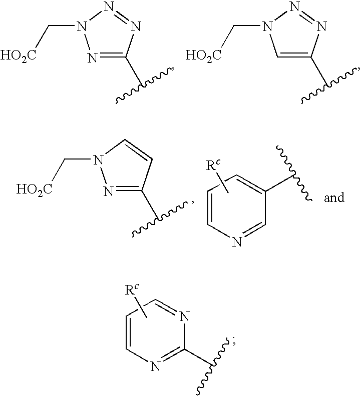

wherein R^{c} is —CO₂H, —CO₂C₁₋₃ alkyl, —CH₂CO₂H, or —CH₂CO₂C₁₋₃ alkyl.

In a class of this embodiment, W is

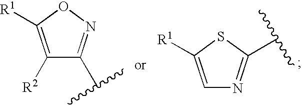

### and R¹ is

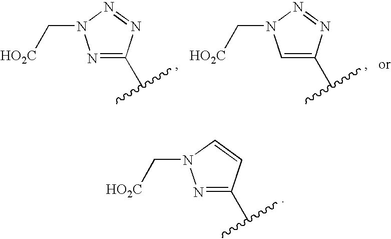

In a subclass of this class, W is

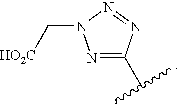

Illustrative, but nonlimiting examples, of compounds of the present invention that are useful as inhibitors of SCD are the following:

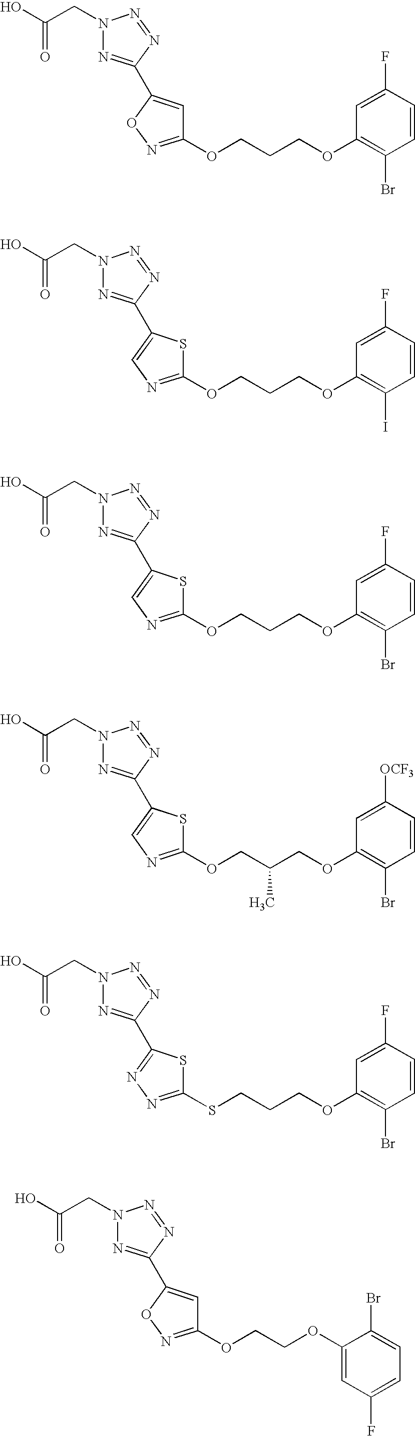

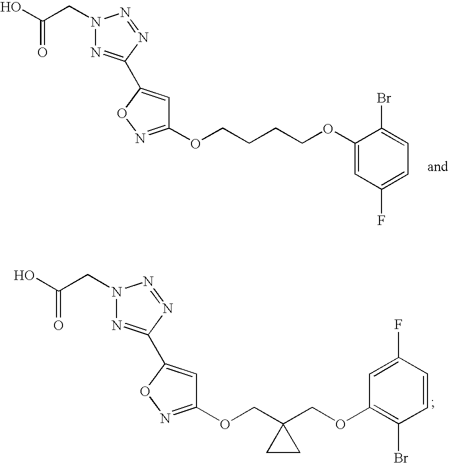

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” shall mean straight or branched-chain alkenes having the specified number of carbon atoms. Examples of alkenyl include vinyl, 1-propenyl, 1-butenyl, 2-butenyl, and the like.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “oxo” refers to a carbonyl oxygen as in C(═O).

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, and 2-oxoazetidin-1-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, and other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD-Induced Rat Liver Microsome Assay:

The activity of compounds of formula I against the SCD enzyme was determined by following the conversion of radiolabeled-stearoyl-CoA to oleoyl-CoA using SCD-induced rat liver microsome and a previously published procedure with some modifications (Joshi, et al., J. Lipid Res., 18: 32-36 (1977)). After feeding wistar rats with a high carbohydrate/fat-free rodent diet (LabDiet #5803, Purina) for 3 days, the SCD-induced livers were homogenized (1:10 w/v) in 250 mM sucrose, 1 mM EDTA, 5 mM DTT and 50 mM Tris-HCl (pH 7.5). After a 20 min centrifugation (18,000×g/4° C.) to remove tissue and cell debris, the microsome was prepared by a 100,000×g centrifugation (60 min) with the resulting pellet suspended in 100 mM sodium phosphate, 20% glycerol and 2 mM DTT. Test compound in 2 μL DMSO was incubated for 15 min. at room temperature with 180 μL of the microsome (typically at about 100 μg/mL, in Tris-HCl buffer (100 mM, pH 7.5), ATP (5 mM), Coenzyme A (0.1 mM), Triton X-100 (0.5 mM) and NADH (2 mM)). The reaction was initiated by the addition of 20 L of [³H]-Stearoyl-CoA (final concentration at 2 μM with the radioactivity concentration at 1 μCi/mL), and terminated by the addition of 150 L of 1N sodium hydroxide. After 60 min at room temperature to hydrolyze the oleoyl-CoA and stearoyl-CoA, the solution was acidified by the addition of 150 L of 15% phosphoric acid (v/v) in ethanol supplemented with 0.5 mg/mL stearic acid and 0.5 mg/mL oleic acid. [³H]-oleic acid and [³H]-stearic acid were then quantified on a HPLC that is equipped with a C-18 reverse phase column and a Packard Flow Scintillation Analyzer. Alternatively, the reaction mixture (80 μL) was mixed with a calcium chloride/charcoal aqueous suspension (100 L of 15% (w/v) charcoal plus 20 μL of 2 N CaCl₂). The resulting mixture was centrifuged to precipitate the radioactive fatty acid species into a stable pellet. Tritiated water from SCD-catalyzed desaturation of 9,10-[³H]-stearoyl-CoA was quantified by counting 50 μL of the supernant on a scintillation counter.

### II. Whole Cell-Based SCD (Delta-9), Delta-5 and Delta-6 Desaturase Assays:

Human HepG2 cells were grown on 24-well plates in MEM media (Gibco cat #11095-072) supplemented with 10% heat-inactivated fetal bovine serum at 37° C. under 5% CO₂ in a humidified incubator. Test compound dissolved in the media was incubated with the subconfluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 M of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 L of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of formula I, particularly the inhibitors of Examples 1 to 66, exhibit an inhibition constant IC₅₀ of less than 1 μM and more typically less than 0.1 μM. Generally, the IC₅₀ ratio for delta-5 or delta-6 desaturases to SCD for a compound of formula I, particularly for Examples 1 to 66 is at least about ten or more, and preferably about one hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase-IV (DPP-4) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CBI receptor inverse agonists and antagonists, (33 adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); vildagliptin (LAF 237); denagliptin; P93/01; saxagliptin (BMS 477118); RO0730699; MP513; SYR-322: ABT-279; PHX1149; GRC-8200; and TS021.

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y₅ antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors;

(u) AMPK activators; and

(v) agonists of GPR-119; and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug.

Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### List of Abbreviations:

Alk=alkyl
APCI=atmospheric pressure chemical ionization
Ar=aryl
Boc=tert-butoxycarbonyl
br=broad
t-BuONO=t-butyl nitrite
d=doublet
DBU=1,8-diazabicyclo[5.4.0]undec-7-ene

### DMF=N,N-dimethylformamide

DIBAL-H=diisobutylaluminum hydride
DMSO=dimethyl sulfoxide
ESI=electrospray ionization
ESMS=electrospray ion-mass spectroscopy
EtOAc=ethyl acetate
HPLC=high-performance liquid chromatography
Hunig's base=N,N-diisopropylethylamine
m=multiplet
mCPBA=m-chloroperbenzoic acid
min=minutes
MeOH=methyl alcohol
MS=mass spectroscopy
NaHMDS=sodium bis(trimethylsilyl)amide
NMP=1-methyl-2-pyrrolidinone
NMR=nuclear magnetic resonance spectroscopy
PG=protecting group
P=pentuplet
Q=quartet
rt=room temperature
s=singlet
t=triplet
TFAA=trifluoroacetic anhydride
Tf₂O=trifluoromethanesulfonic anhydride
THF=tetrahydrofuran
TLC=thin-layer chromatography
TsOH=toluene-4-sulfonic acid

### Preparation of Compounds of the Invention:

The compounds of structural formula I can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### Method A:

An appropriately substituted heteroaryl amine 1 is reacted with t-butyl nitrite and anhydrous copper (II) halide in a solvent such as acetonitrile to give heteroaryl halide 2. Treatment of 2 with ammonia in a solvent such as THF gives amide 3. Dehydration with TFAA or Tf₂O in a solvent such as CH₂ Cl₂ gives the nitrile intermediate 4.

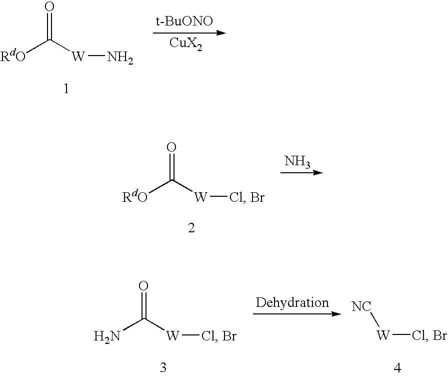

### Method B:

An appropriately substituted amino-heteroaryl halide is reacted with t-butyl nitrite and anhydrous cuprous cyanide in a solvent such as acetonitrile to give the nitrile intermediate 4.

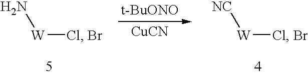

### Method C:

The nitrile intermediate 4 is reacted with an appropriately substituted nucleophile in the presence of a base such as DBU or an alkali metal (K, Na, Cs) carbonate in a solvent such as THF, 1,4-dioxane, and DMF at a temperature range of about room temperature to refluxing temperature. Extractive work-up and purification by flash column chromatography gives the condensed product 6.

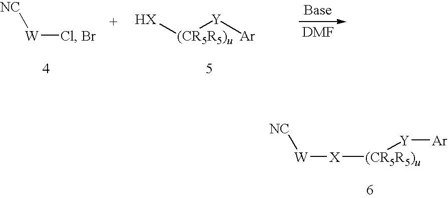

### Method D:

The ester intermediate 7 is reacted with an appropriately substituted electrophile in the presence of a base such as DBU or an alkali metal (K, Na, Cs) carbonate in a solvent such as THF, 1,4-dioxane, and DMF at a temperature range of about room temperature to refluxing temperature. Extractive work-up and purification by flash column chromatography gives the condensed product 9.

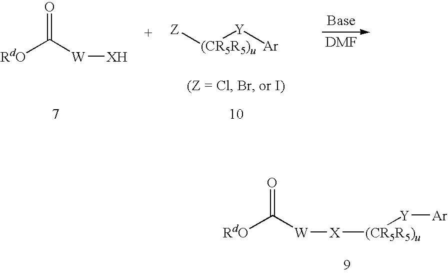

### Method E:

The ester intermediate 7 (X═O) is reacted with an appropriate heteroaryl alcohol intermediate 5 (X═O) under Mitsunobu conditions (an azodicarboxylate, such as diethyl azodicarboxylate, in the presence of a phosphine, such as triphenylphosphine). Extractive work-up and purification by flash column chromatography gives the condensed product 9 (X═O).

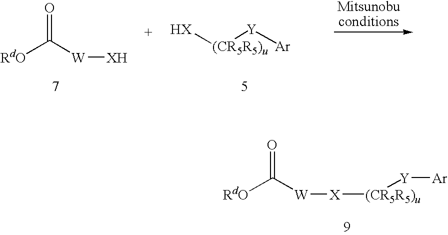

### Method F:

The ester intermediate 7 is reacted with an appropriate electrophile 11 in the presence of a base such as DBU or an alkali metal (K, Na, Cs) carbonate in a solvent such as THF, 1,4-dioxane, and DMF at a temperature range of about room temperature to refluxing temperature. Extractive work-up and purification by flash column chromatography gives the condensed product 12. The ester intermediate 12 is then reacted with an appropriate nucleophile 13 in the presence of a base such as DBU or an alkali metal (K, Na, Cs) carbonate in a solvent such as THF, 1,4-dioxane, and DMF at a temperature range of about room temperature to refluxing temperature. Extractive work-up and purification by flash column chromatography gives the condensed product 9.

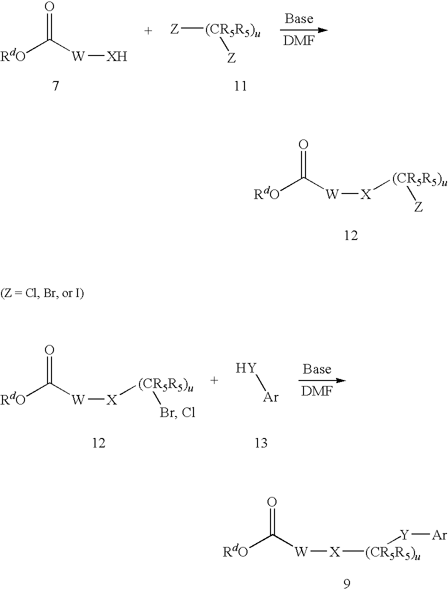

### Method G:

The ester intermediate 9 prepared according to Method D, E or F is reacted with ammonia in a solvent such as THF to give amide 14. Alternatively, the amide 14 can be prepared by reacting the ester intermediate 9 with ammonia in MeOH. Dehydration with TFAA or Tf20 in a solvent such as CH₂Cl₂ gives the nitrile intermediate 6.

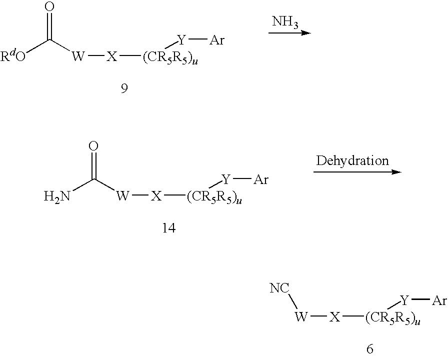

### Method H:

The nitrile intermediate 6 prepared according to Method C or G is reacted with NaN₃ in the presence of a Lewis acid catalyst, such as pyridinium hydrochloride, in a solvent such as NMP, or with NaN₃ in the presence of a Lewis acid catalyst, such as ZnBr₂, in a solvent such as 2-propanol and water to give the tetrazole intermediate 15. Alkylation with a haloalkanoic acid ester, such as ethyl bromoacetate, in the presence of a base such as Cs₂ CO₃ or KOt-Bu in a solvent such as DMF usually gives a mixture of 16 and 17, which can be separated by chromatography. Hydrolysis of the ester groups in 16 and 17 under alkaline conditions, such as with aqueous sodium hydroxide, in a solvent such as THF with an alcoholic solvent such as MeOH, at a temperature range of about room temperature to refluxing gives the carboxylic acids 18 and 19.

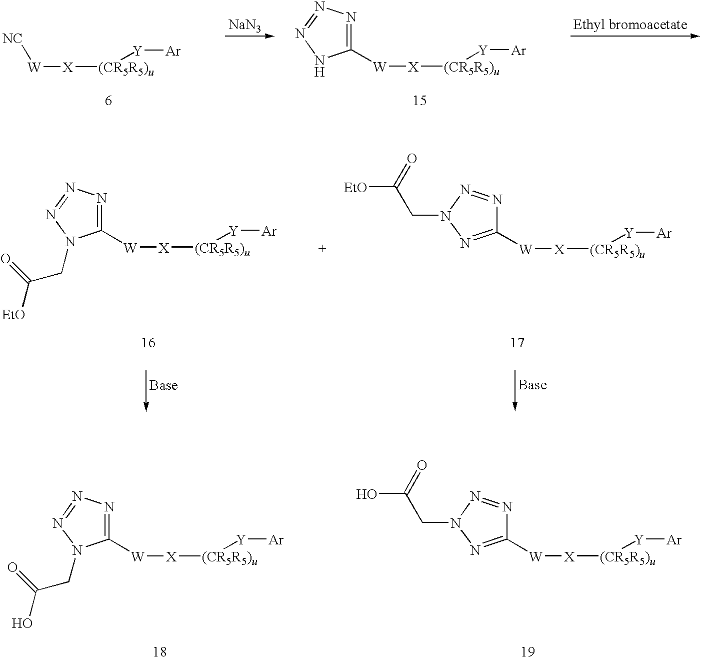

The following methods (Method I, J, K and L) describe an alternative route for the preparation of Intermediate 17.

### Method I:

The tetrazole intermediate 22 is deprotected in the presence of an acid such as TFA and a nucleophile such as dimethylsulfide in a solvent such as the mixture of water and CH₂ Cl₂ at a temperature such as room temperature. Removal of solvents under vacuum at low temperature followed by purification under trituration with an appropriate solvent such as water and toluene gives the cleaved product 20.

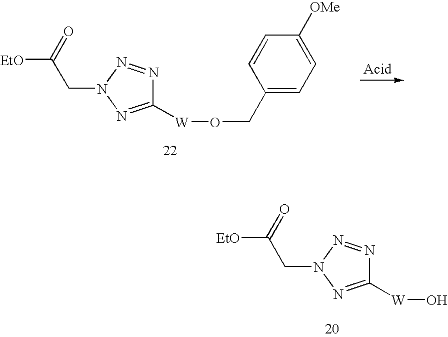

### Method J:

The ester intermediate 20 is reacted with an appropriately substituted electrophile in the presence of a base such as DBU or an alkali metal (K, Na, Cs) carbonate in a solvent such as THF, 1,4-dioxane, and DMF at a temperature range of about room temperature to refluxing temperature. Extractive work-up and purification by column chromatography gives the condensed product 17 (X═O).

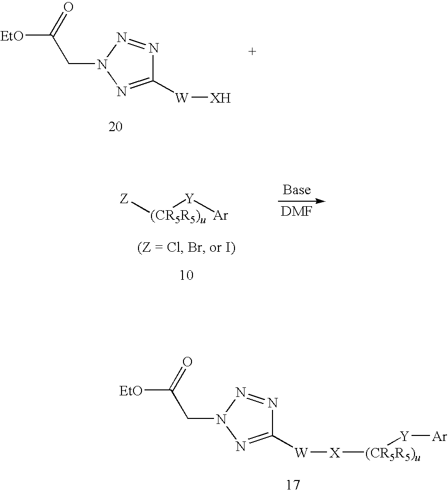

### Method K:

The ester intermediate 20 (X═O) is reacted with an appropriate aryl alcohol intermediate 5 (X═O) under Mitsunobu conditions. Extractive work-up and purification by flash column chromatography gives the condensed product 17 (X═O).

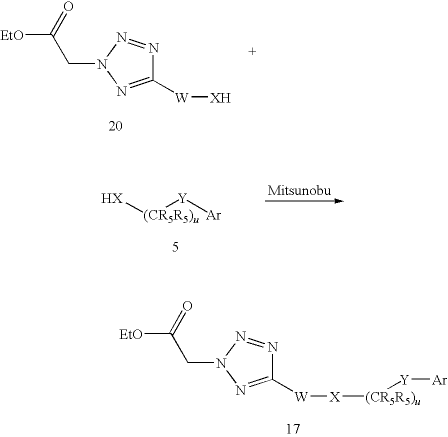

### Method L:

The ester intermediate 20 is reacted with an appropriate electrophile 11 in the presence of a base such as DBU or an alkali metal (K, Na, Cs) carbonate in a solvent such as THF, 1,4-dioxane, and DMF at a temperature range of about room temperature to refluxing temperature. Extractive work-up and purification by column chromatography gives the condensed product 21. The ester intermediate 21 is then reacted with an appropriate nucleophile 13 in the presence of a base such as DBU or an alkali metal (K, Na, Cs) carbonate in a solvent such as THF, 1,4-dioxane, and DMF at a temperature range of about room temperature to refluxing temperature. Extractive work-up and purification by column chromatography gives the condensed product 17.

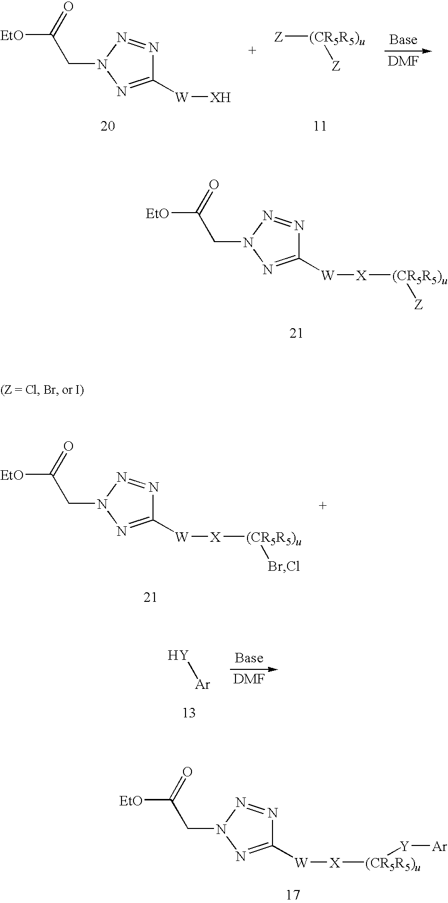

### Method M:

A cyclic diol 23 is reacted with an appropriately substituted aryl fluoride 24 in the presence of a base, such as sodium hydride and potassium carbonate in a solvent, such as DMF and THF under reflux conditions to afford the ether derivative 25. Reaction of the ether derivative 25 with the hydroxyheteroarene derivative 26 under standard Mitsunobu conditions with triphenyl phosphine, di-tert-butyl azodicarboxylate or di-ethyl azodicarboxylate in a solvent such as THF or toluene at about room temperature or under reflux conditions gives the heteroaryl ester 27. Hydrolysis of the heteroaryl ester 27 with aqueous NaOH or LiOH in a solvent such as THF and MeOH at a temperature range of about room temperature to about refluxing temperature followed by extractive work up and purification by flash column chromatography or recrystallization affords the heteroaryl carboxylic acid 28.

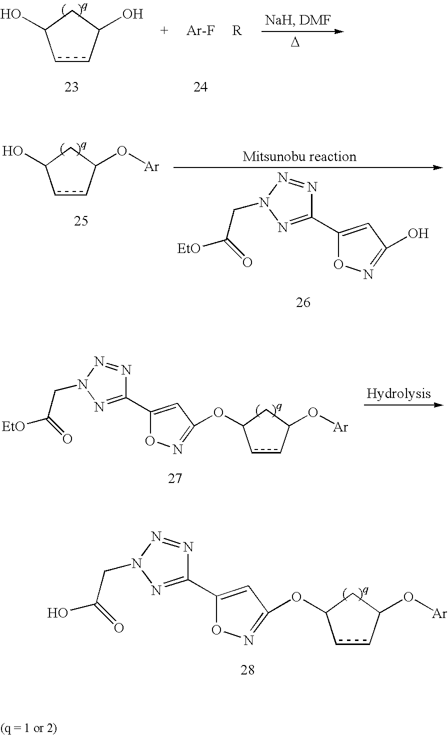

### Method N:

The nitrile intermediate 6 prepared according to Method C or G is reacted first with LiHMDS in a solvent such as DMF to give the carboximidamide intermediate 29 in situ.

Formation of the pyrimidine ring of intermediate 30 is accomplished according to the literature conditions described by P. Zhichkin et al. (Synthesis 2002, 6, 720-722) by using sodium 3,3-dimethoxy-2-carbomethoxyprop-1-ene-1-oxide (Zhichkin, P.; Fairfax, D. J.; Eisenbeis, S. A. Synthesis 2002, 6, 720-722) and a proton source such as NH₄Cl in an appropriate solvent such as DMF. Hydrolysis of the ester group in 30 is performed under alkaline conditions, such as with aqueous sodium hydroxide, in a solvent such as THF with an alcoholic solvent such as MeOH, at a temperature range of about room temperature to refluxing temperature affords the carboxylic

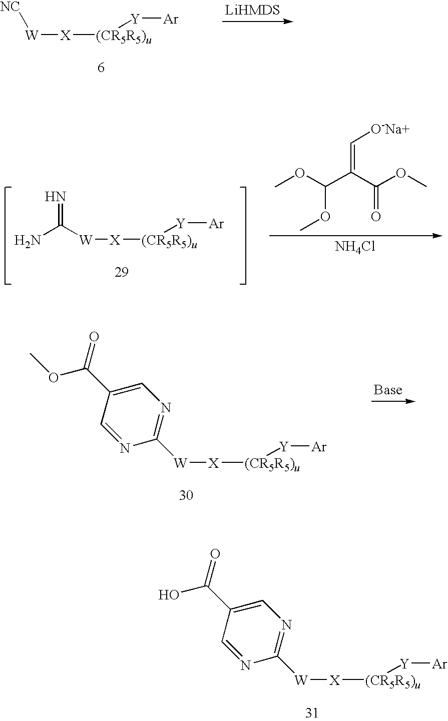

### Method O:

The ester intermediate 32 is reacted with an appropriately substituted electrophile 10 in the presence of a base such as DBU or an alkali metal (K, Na, Cs) carbonate in a solvent such as THF, 1,4-dioxane, and DMF at a temperature range of about room temperature to refluxing temperature. Extractive work-up and purification by flash column chromatography gives the condensed product 9.

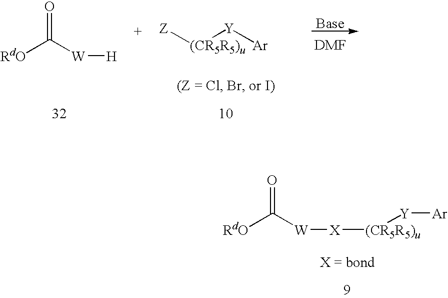

### Preparation of Intermediates

### Intermediate 1

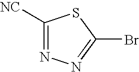

### 5-Bromo-1,3,4-thiadiazole-2-carbonitrile

### Step 1: Ethyl 5-bromo-1,3,4-thiadiazole-2-carboxylate

To a suspension of ethyl 5-amino-1,3,4-thiadiazole-2-carboxylate in CH₃ CN (0.32 M) was added CuBr₂ (2 equiv). The mixture turned dark green and was stirred for 15 min at room temperature. t-BuONO, 90% (2 equiv) was added dropwise over 15-20 min. The mixture became slightly warm and gas was evolved after about 5 min and then throughout the addition. After completion of the addition and gas evolution subsided, the mixture was heated at 60° C. for min. Solvent was then evaporated under diminished pressure. Water and EtOAc were added and the mixture was stirred until the dark green color disappeared. The organic phase became light brown and the aqueous phase was green with insoluble material. The entire mixture was filtered through celite and washed with EtOAc. The EtOAc layer was separated, washed with diluted brine, dried (Na₂SO₄) and concentrated to give the title compound. ¹H NMR (400 MHz, acetone-d₆): δ 4.52 (q, 2H), 1.43 (t, 3H).

### Step 2: 5-Bromo-1,3,4-thiadiazole-2-carboxamide

To a solution of ethyl 5-bromo-1,3,4-thiadiazole-2-carboxylate in THF (1.1 M) at room temperature was added concentrated NH₄ OH (2.9 equiv). The mixture was stirred at room temperature overnight and a precipitate appeared in the aqueous layer. Volatile solvent was removed under diminished pressure. The mixture was diluted with water and the precipitate was collected, washed with water and dried under vacuum to give the title compound. ¹H NMR (400 MHz, acetone-d₆): δ 7.99 (s, 1H), 7.55 (s, 1H).

### Step 3: 5-Bromo-1,3,4-thiadiazole-2-carbonitrile

To a solution of 5-bromo-1,3,4-thiadiazole-2-carboxamide and Et₃ N (2.3 equiv) in THF (0.5 M) at 0° C. was added TFAA (1.1 equiv). The mixture was then warmed to room temperature and stirred for 30 min. Solvent was evaporated under diminished pressure. The residue was diluted with water. The precipitate was collected, washed with water, and dried to give the title compound.

### Intermediate 2

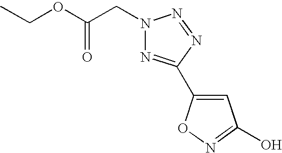

### Ethyl[5-(3-hydroxyisoxazol-5-yl)-2H-tetrazol-2-yl]acetate

### Step 1: Methyl 3-[(4-methoxybenzyl)oxy]isoxazole-5-carboxylate

To a solution of methyl 3-hydroxyisoxazole-5-carboxylate (20.1055 g, 141 mmol) in DMF (100 mL) at 0° C. was added potassium carbonate (22.0119 g, 159 mmol), and after 10 min 4-methoxybenzyl chloride (23 mL, 169 mmol). The yellow suspension was stirred 15 min at 0° C., 15 min at room temperature and 1.5 h at 60° C. The reaction mixture was poured into aqueous 1N HCl, extracted with EtOAc and washed four times with 1N HCl and brine. The organic layer was dried (Na₂SO₄) and filtered. Solvents were removed under diminished pressure to afford the crude product. The crude product was purified by column chromatography on silica gel (gradient 10-30% EtOAc/hexanes) to afford the title compound as a colorless oil. ¹H NMR (500 MHz, acetone-d₆): δ 7.50-7.44 (m, 2H), 7.01-6.97 (m, 2H), 6.80 (s, 1H), 5.27 (s, 2H), 3.94 (s, 3H), 3.83 (s, 3H).

### Step 2: 3-[(4-Methoxybenzyl)oxy]isoxazole-5-carboxamide

To a solution of methyl 3-[(4-methoxybenzyl)oxy]isoxazole-5-carboxylate (24.5 g, 93 mmol) in THF (40 mL) was added concentrated ammonium hydroxide (100 mL, 719 mmol) at 0° C. The final suspension was warmed and stirred at room temperature for 2 d. Water was added to the reaction mixture and the precipitate was collected by filtration and dried under high vacuum to afford the title compound as a white solid. ¹H NMR (400 MHz, DMSO-d₆): δ 8.27 (br s, 1H), 7.95 (br s, 1H), 7.43 (d, 2H), 6.97 (d, 2H), 6.81 (s, 1H), 5.21 (s, 2H), 3.78 (s, 3H).

### Step 3: 3-[(4-Methoxybenzyl)oxy]isoxazole-5-carbonitrile

To a suspension of 3-[(4-methoxybenzyl)oxy]isoxazole-5-carboxamide (20.21 g, 81 mmol) and N,N-diisopropylethylamine (140 mL, 802 mmol) in CH₂Cl₂ (200 mL) was added dropwise trifluoroacetic anhydride (16 mL, 113 mmol) at −78° C. The solution was warmed slowly to 0° C. and TLC indicated that the reaction was over. The reaction mixture was then poured into aqueous ammonium chloride, extracted with EtOAc and washed with brine. The organic layer was dried (Na₂SO₄) and filtered. Solvents were removed under diminished pressure to afford the crude product. The crude material was purified by column chromatography on silica gel (gradient from 0 to 30% EtOAc/hexanes) to afford the title compound as a pale yellow solid. ¹H NMR (500 MHz, acetone-d₆): δ 7.48 (d, 2H), 7.18 (s, 1H), 6.99 (d, 2H), 5.31 (s, 2H), 3.84 (s, 3H).

### Step 4: 5-{3-[(4-Methoxybenzyl)oxy]lisoxazol-5-yl}-1H-tetrazole

A suspension of 3-[(4-methoxybenzyl)oxy]isoxazole-5-carbonitrile (20.55 g, 89 mmol), sodium azide (29.0 g, 446 mmol) and pyridine hydrochloride (20.63 g, 179 mmol) (dried by heating under vacuum) in NMP (248 ml) was heated to 140° C. for 1.5 h. The reaction mixture was diluted with EtOAc (1 L) and 1N HCl (1.5 L). The organic phase was separated and washed with 1N HCl (5×500 mL), brine (500 mL) and dried (Na₂SO₄). The first aqueous phase was extracted again with EtOAc (3×500 mL) and washed with aqueous phases as described above. The organic phases were combined and concentrated to give the title compound as a beige solid. ¹H NMR (500 MHz, DMSO-d₆): δ 7.46 (d, 2H), 7.08 (s, 1H), 6.98 (d, 2H), 5.27 (s, 2H), 3.78 (s, 3H).

### Step 5: Ethyl(5-{3-[(4-methoxybenzyl)oxy]isoxazol-5-yl}-2H-tetrazol-2-yl)acetate & ethyl(5-{3-[(4-methoxybenzyl)oxy]isoxazol-5-yl}-1H-tetrazol-1-yl)acetate (ratio 4:1)

To a solution of 5-{3-[(4-methoxybenzyl)oxy]isoxazol-5-yl}-1H-tetrazole (24.3 g, 89 mmol) in 1,4-dioxane (450 mL) was added N,N-diisopropylethylamine (50 mL, 286 mmol) and ethyl bromoacetate (20 mL, 180 mmol). The reaction was heated at 90° C. for 1 h. The reaction mixture was poured into 1N HCl, extracted twice with EtOAc and washed with brine. The organic layer was dried (Na₂SO₄) and filtered. Solvents were removed under diminished pressure to afford the crude product. The crude material was purified by column chromatography on silica gel (gradient from 0 to 30% EtOAc/hexanes). The material was triturated with ether/hexanes to afford the title compound as a beige solid (regioisomeric ratio 4:1).

Major isomer: ¹H NMR (400 MHz, acetone-d₆): δ 7.54-7.48 (m, 2H), 7.03-6.96 (m, 2H), 6.86 (s, 1H), 5.85 (s, 2H), 5.32 (s, 2H), 4.31 (q, 2H), 3.85 (s, 3H), 1.31 (t, 3H).

Minor isomer: ¹H NMR (400 MHz, acetone-d₆): δ 7.54-7.48 (m, 2H), 7.03-6.96 (m, 3H), 5.79 (s, 2H), 5.32 (s, 2H), 4.34-4.26 (m, 2H), 3.85 (s, 3H), 1.34-1.22 (m, 3H).

### Step 6: Ethyl[5-(3-hydroxyisoxazol-5-yl)-2H-tetrazol-2-yl]acetate

To a solution of a mixture of ethyl(5-{3-[(4-methoxybenzyl)oxy]isoxazol-5-yl}-2H-tetrazol-2-yl)acetate & ethyl(5-{3-[(4-methoxybenzyl)oxy]isoxazol-5-yl}-1H-tetrazol-1-yl)acetate (ratio 4:1) (14.5 g, 40.4 mmol) in CH₂Cl₂ (200 mL) was added dimethyl sulfide (35 mL, 473 mmol), water (35 mL, 1943 mmol) and TFA (100 mL, 1298 mmol) at 0° C. The reaction was stirred at room temperature for 3 h. The reaction mixture was concentrated to 5-10 mL of volume (water-TFA mixture) under vacuum by keeping the external temperature below 40° C. Water (200 mL) was added and the precipitate was filtered and washed with water (3×50 mL). The precipitate was triturated with hot toluene (750 mL) and cooled to 0° C. before filtration to afford the title compound as a white solid. ¹H NMR (400 MHz, acetone-d₆): δ 10.48 (br s, 1H), 6.77 (s, 1H), 5.84 (s, 2H), 4.31 (q, 2H), 1.31 (t, 3H). MS: m/z 240.0 (MH+).

### Intermediate 3

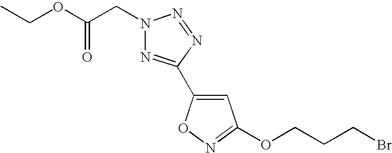

### Ethyl{5-[3-(3-bromopropoxy)isoxazol-5-yl]-2H-tetrazol-2-yl}acetate

To a solution of ethyl[5-(3-hydroxyisoxazol-5-yl)-2H-tetrazol-2-yl]acetate (INTERMEDIATE 2) (1 g, 4.18 mmol) in DMF (5.00 mL) at 0° C. was added potassium carbonate (0.636 g, 4.60 mmol) and 1,3-dibromopropane (2.2 mL, 21.67 mmol). The yellow suspension was warmed slowly to room temperature and heated to 60° C. for 1.5 h. The reaction mixture was poured into aqueous 1N HCl, extracted with EtOAc and washed with brine. The organic layer was dried (Na₂SO₄) and filtered. Solvents were removed under diminished pressure to afford the crude product which was purified by column chromatography on silica gel (gradient 10-50% EtOAc/hexanes) followed by a trituration with ether/heptane to afford the title compound as a white solid. ¹H NMR (400 MHz, acetone-d₆): δ 6.87 (s, 1H), 5.86 (s, 2H), 4.51 (t, 2H), 4.31 (q, 2H), 3.71 (t, 2H), 2.45-2.41 (m, 2H), 1.31 (t, 3H).

### Intermediate 4

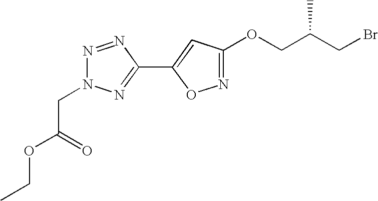

### Ethyl[5-(3-{[(2S)-3-bromo-2-methylpropy]oxy}isoxazol-5-yl)-2H-tetrazol-2-yl]acetate

To a solution of (2S)-3-bromo-2-methylpropan-1-ol (108 mg, 0.706 mmol) and ethyl[5-(3-hydroxyisoxazol-5-yl)-2H-tetrazol-2-yl]acetate (INTERMEDIATE 2) (100 mg, 0.418 mmol) in THF (2 mL) was added di-tert-butyl azodicarboxylate (130 mg, 0.565 mmol). The yellow solution was cooled to −78° C. and treated with a solution of triphenylphosphine (152 mg, 0.580 mmol) in CH₂Cl₂ (2 mL). The final mixture was warmed and stirred overnight at room temperature. Solvents were removed under diminished pressure to afford the crude product. The crude material was purified by column chromatography on silica gel (gradient from 0 to 60% EtOAc/hexanes) to afford the title compound as a white solid.

¹H NMR (400 MHz, acetone-d₆): δ 6.89 (s, 1H), 5.86 (s, 2H), 4.36-4.28 (m, 4H), 3.69 (dd, 2H), 2.49-2.43 (m, 1H), 1.31 (t, 3H), 1.19 (d, 3H).

### Intermediate 5

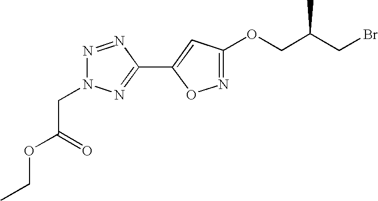

### Ethyl[5-(3-{[(2R)-3-bromo-2-methylpropyl]oxy}isoxazol-5-yl)-2H-tetrazol-2-yl]acetate

The title compound was prepared in a similar manner as that described for intermediate 4 from ethyl[5-(3-hydroxyisoxazol-5-yl)-2H-tetrazol-2-yl]acetate (INTERMEDIATE 2) and (2R)-3-bromo-2-methylpropan-1-ol.

### Intermediate 6

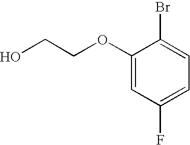

### 2-(2-Bromo-5-fluorophenoxy)ethanol

A mixture of 2-bromo-5-fluorophenol (1.0067 g, 5.27 mmol), ethylene carbonate (477 mg, 5.42 mmol) and imidazole (11 mg, 0.162 mmol) was immersed into a preheated oil bath at 150° C. The reaction was maintained at this temperature for 5 h. The crude product was purified by column chromatography on silica gel (gradient: 10-50% EtOAc/hexanes) to afford the title compound as a colorless oil. ¹H NMR (500 MHz, acetone-d₆): δ 7.59 (dd, 1H), 6.99 (dd, 1H), 6.72 (td, 1H), 4.21 (t, 2H), 4.03 (t, 1H), 3.95 (q, 2H). MS: m/z 236.8, 235.0 (MH+).

### Intermediate 7

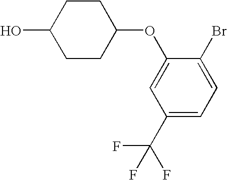

### 4-[2-Bromo-5-(trifluoromethyl)phenoxy]cyclohexanol

To a solution of 4-bromo-3-fluorobenzotrifluoride (2 g, 8.23 mmol) and a mixture of cis and trans cyclohexane-1,4-diol (3.82 g, 32.9 mmol) in DMF (41.2 ml) was added NaH (0.658 g, 16.46 mmol) at 0° C. The reaction mixture was warmed to room temperature then heated at 80° C. for 2 h. The mixture was poured onto 1N HCl (100 mL) and extracted with EtOAc (3×25 mL). The combined organic fractions were washed with water (50 mL) then dried over Na₂SO₄. Purification by Combiflash chromatography (SiO₂—40 g, gradient elution of 10-50% EtOAc/hexanes over 25 min) afforded the title product as a 7:3 mixture of isomers.

Major isomer: ¹H NMR (500 MHz, acetone-d₆): δ 7.82 (dd, 1H), 7.43 (d, 1H), 7.23 (d, 1H), 4.76-4.70 (m, 1H), 3.84-3.76 (m, 1H), 3.67 (d, 1H), 2.18-2.12 (m, 1H), 2.06-1.95 (m, 1H), 1.82-1.71 (m, 2H), 1.69-1.60 (m, 2H), 1.55-1.47 (m, 2H). MS: m/z 339, 341 (MH⁺).

### Intermediate 8

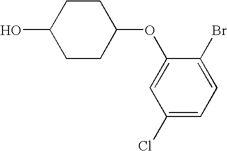

### 4-(2-Bromo-5-chlorophenoxy)cyclohexanol

The title compound was prepared in a similar manner as that described for Intermediate 7 from 1-bromo-4-chloro-2-fluorobenzene, a mixture of cis and trans-cyclohexane-1,4-diol and sodium hydride. The product was obtained as a 7:3 mixture of isomers.

Major isomer: ¹H NMR (500 MHz, acetone-d₆): δ 7.58 (dd, 1H), 7.21 (d, 1H), 6.93 (dd, 1H), 4.63-4.57 (m, 1H), 3.82-3.75 (m, 1H), 3.69 (d, 1H), 2.15-2.10 (m, 1H), 2.02-1.95 (m, 1H), 1.83-1.70 (m, 2H), 1.66-1.58 (m, 2H), 1.54-1.46 (m, 2H). MS: m/z 305, 307 (MH⁺).

### Intermediate 9

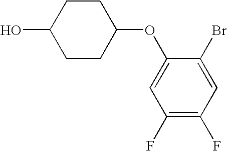

### 4-(2-Bromo-4,5-difluorophenoxy)cyclohexanol

The title compound was prepared in a similar manner as that described for Intermediate 7 from 1-bromo-2,4,5-trifluorobenzene, a mixture of cis and trans-cyclohexane-1,4-diol and sodium hydride. The product was obtained as a 7:3 mixture of isomers.

Major isomer: ¹H NMR (500 MHz, acetone-d₆): δ 7.53-7.47 (m, 1H), 7.30-7.20 (m, 1H), 4.57-4.44 (m, 1H), 3.85-3.67 (m, 2H), 2.17-2.10 (m, 2H), 2.04-1.94 (m, 1H), 1.80-1.67 (m, 2H), 1.64-1.54 (m, 2H), 1.53-1.42 (m, 2H). MS: m/z 305, 307 (MH⁺).

### Intermediate 10

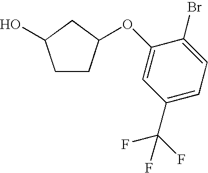

### 3-[2-Bromo-5-(trifluoromethyl)phenoxy]cyclopentanol

The title compound was prepared in a similar manner as that described for Intermediate 7 from 4-bromo-3-fluorobenzotrifluoride, a mixture of cis and trans cyclopentane-1,3-diol and sodium hydride. The product was obtained as a 7:3 mixture of isomers.

Major isomer: MS: m/z 325, 327 (MH⁺).

### Intermediate 11

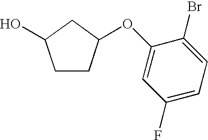

### 3-(2-Bromo-5-fluorophenoxy)cyclopentanol

The title compound was prepared in a similar manner as that described for Intermediate 7 from 1-bromo-2,4-difluorobenzene, a mixture of cis and trans-cyclopentane-1,3-diol and sodium hydride. The product was obtained as a 7:3 mixture of isomers.

Major isomer: ¹H NMR (500 MHz, acetone-d₆): δ 7.61-7.55 (m, 1H), 6.94-6.88 (m, 1H), 6.70 (td, 1H), 5.07-5.04 (m, 1H), 4.50-4.47 (m, 1H), 3.78 (d, 1H), 2.34-2.24 (m, 1H), 2.12-2.00 (m, 3H), 1.86-1.74 (m, 1H), 1.72-1.64 (m, 1H). MS: m/z 325, 327 (MH⁺).

### Intermediate 12

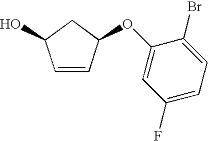

### Cis-4-(2-Bromo-5-fluorophenoxy)cyclopent-2-en-1-ol

The title compound was prepared in a similar manner as that described for Intermediate 7 from 1-bromo-2,4-difluorobenzene, cis-cyclopent-4-ene-1,3-diol and sodium hydride. ¹H NMR (500 MHz, acetone-d₆): δ 7.60 (dd, 1H), 7.02 (dd, 1H), 6.72 (td, 1H), 6.15 (d, 1H), 6.08 (d, 1H), 5.30 (t, 1H), 4.80-4.74 (m, 1H), 4.23 (d, 1H), 3.07 (dt, 1H), 1.71 (dt, 1H).

### Intermediate 13

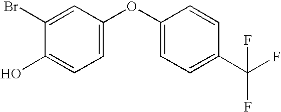

### 2-Bromo-4-[4-(trifluoromethyl)phenoxy]phenol

To a suspension of 4-[4-(trifluoromethyl)phenoxy]phenol (1.02 g, 4.02 mmol) in acetic acid (8 mL) at room temperature was slowly added bromine (217 μL, 4.22 mmol) dropwise over 30 min. The resulting solution was stirred for 2.5 h. The reaction mixture was then carefully partitioned between EtOAc and NaHCO₃, the organic layer was dried over Na₂SO₄ and concentrated. The resulting crude product was purified on a 120-g silica gel cartridge eluted with EtOAc in hexanes going from 5 to 25% over 28 min@80 mL/min to give the title compound as a colorless oil.

¹H NMR (400 MHz, acetone-d₆): δ 9.01 (s, 1H), 7.73-7.68 (m, 2H), 7.34 (d, 1H), 7.15-7.09 (m, 3H), 7.07-7.03 (m, 1H).

### Intermediate 14

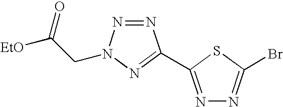

### Ethyl[5-(5-bromo-1,3,4-thiadiazol-2-yl)-2H-tetrazol-2-yl]acetate

To a suspension of 5-bromo-1,3,4-thiadiazole-2-carbonitrile (1 g, 5 mmol) and ZnBr₂ (1.1 g, 5 mmol) in i-PrOH (10 mL) and H₂O (5 mL) was added NaN₃ (0.65 g, 10 mmol) in a sealed tube. The mixture was stirred at 120° C. overnight and cooled to room temperature. The mixture was adjusted to pH 4 with 2N HCl and extracted with EtOAc (50 mL×3). The combined organic layers were dried over Na₂SO₄, filtered and concentrated under diminished pressure to afford the crude 5-(5-bromo-1,3,4-thiadiazol-2-yl)-1H-tetrazole. ¹³C NMR (DMSO, 300 MHz): δ 159.12, 150.65, 142.84.

To a solution of 5-(5-bromo-1,3,4-thiadiazol-2-yl)-1H-tetrazole (1 g, 4.3 mmol) in DMF (20 mL) was added Cs₂ CO₃ (2.1 g, 6.45 mmol) and ethyl bromoacetate (0.95 mL, 8.6 mmol). The resulting solution was stirred at 90° C. for 1 h. The mixture was partitioned between EtOAc (100 mL) and water (200 mL). The combined organic layers were dried over anhydrous Na₂SO₄, filtered and evaporated under vacuum. Chromatography over silica afforded the title compound as a white solid, contaminated with the 1-alkylated isomer ethyl[5-(5-bromo-1,3,4-thiadiazol-2-yl)-1H-tetrazol-1-yl]acetate.

¹HNMR (CDCl₃, 300 MHz): δ 5.70 (s, 2H), 4.26 (q, J=7 Hz, 2H), 1.28 (t, J=7 Hz, 3H).

### Intermediate 15

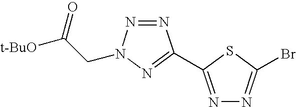

### tert-Butyl[5-(5-bromo-1,3,4-thiadiazol-2-yl)-2H-tetrazol-2-yl]acetate

The title compound was prepared in a similar manner as described for Intermediate 14 from 5-(5-bromo-1,3,4-thiadiazol-2-yl)-1H-tetrazole and tert-butyl bromoacetate, contaminated with about 20% of tert-butyl[5-(5-bromo-1,3,4-thiadiazol-2-yl)-1H-tetrazol-1-yl]acetate. ¹HNMR (CDCl₃ 300 MHz): δ 5.43 (s, 2H), 1.47 (s, 9H).

### Intermediate 16

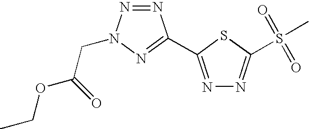

### Ethyl{5-[5-(methylsulfonyl)-1,3,4-thiadiazol-2-yl]-2H-tetrazol-2-yl}acetate

### Step 1: 5-(Methylthio)-1,3,4-thiadiazole-2-carboxamide

To a solution of 5-bromo-1,3,4-thiadiazole-2-carboxamide (5 g, 24.03 mmol) in EtOH (80 mL) was added NaSMe (2.021 g, 28.8 mmol). The reaction mixture was stirred at room temperature for 2.5 h. The reaction mixture was diluted with water (40 mL) and the precipitate was filtered and washed with water. The filtrate was evaporated under reduced pressure and extracted with EtOAc (3×20 mL). The combined organic layers were dried (MgSO₄), filtered and evaporated under reduced pressure. The product was triturated overnight in Et₂O, then filtered to afford the title compound as a solid.

¹H NMR (400 MHz, acetone-d₆): δ 7.79 (s, 1H), 7.30 (s, 1H), 2.76 (s, 3H). MS (+ESI) m/z 176 (MH⁺).

### Step 2: 5-(Methylthio)-1,3,4-thiadiazole-2-carbonitrile

To a solution of 5-(methylthio)-1,3,4-thiadiazole-2-carboxamide (3.4 g, 19.2 mmol) and triethylamine (8.0 mL, 57.5 mmol) in CH₂ Cl₂ (75 mL) was added TFAA (4.1 mL, 28.8 mmol) at 0° C. After 5 min the mixture was warmed to room temperature and stirred for a further 1 h. The solvent was evaporated and the residue was diluted with water (25 mL). The aqueous layer was extracted with EtOAc (3×20 mL). The combined organic fractions were dried over MgSO₄ and the solvent was evaporated under reduced pressure. The product was triturated with Et₂O/Hexanes to afford the title product as a solid.

¹H NMR (500 MHz, acetone-d₆): δ 2.92 (s, 3H). MS (+ESI) m/z 158 (MH⁺).

### Step 3: 5-[5-(Methylthio)-1,3,4-thiadiazol-2-yl]-2H-tetrazole

A suspension of 5-(methylthio)-1,3,4-thiadiazole-2-carbonitrile (2.4 g, 15.1 mmol), NaN₃ (4.9 g, 76 mmol) and pyridinium hydrochloride (3.5 g, 30.3 mmol) in NMP (35 mL) was heated at 130° C. for 2 h. The reaction mixture was cooled to room temperature, diluted with water (30 mL) and extracted with EtOAc (2×15 mL). The aqueous layer was acidified to pH 1 with 1N HCl and extracted with EtOAc (10×20 mL). The combined organic layers were dried (MgSO₄), filtered and evaporated under reduced pressure to afford title compound.

¹H NMR (500 MHz, acetone-d₆): δ 2.72 (s, 3H).

### Step 4: 5-[5-(methylthio)-1,3,4-thiadiazol-2-yl]-2H-tetrazole-H-tetrazol-2yl}acetate

A mixture of 5-[5-(methylthio)-1,3,4-thiadiazol-2-yl]-2H-tetrazole (3 g, 15 mmol), triethylamine (4.2 mL, 30 mmol), ethyl bromoacetate (2.5 mL, 22.5 mmol) in THF (25 mL) was heated at 80° C. for 2 h. The solvent was evaporated, the residue was diluted with water (15 mL) and extracted with EtOAc (3×15 mL). The combined organic fractions were dried over MgSO₄. The solvent was evaporated under reduced pressure and purification by Combiflash chromatography (SiO₂—120 g, gradient elution of 20-40% EtOAc/hexanes over 30 min) afforded the title product as the more polar isomer.

¹H NMR (500 MHz, acetone-d₆): δ 5.84 (s, 2H), 4.28 (q, 2H), 2.90 (s, 3H), 1.28 (t, 3H). MS (+ESI) m/z 287 (MH⁺).

### Step 5: Ethyl{5-[5-(methylsulfonyl)-1,3,4-thiadiazol-2-yl]-2H-tetrazol-2-yl}acetate

To a solution of ethyl 5-[5-(methylthio)-1,3,4-thiadiazol-2-yl]-2H-tetrazole-H-tetrazol-2-yl}acetate (2.6 g, 9.1 mmol) in chloroform (45 mL) was added mCPBA (8 g, 25.5 mmol). The reaction mixture was stirred at room temperature for 1 h. Additional mCPBA was added and the reaction mixture was further stirred for 1 h. The reaction mixture was diluted with chloroform (20 mL) and washed with 0.5N NaOH (2×) and brine. The organic layer was dried (MgSO₄) and evaporated under reduced pressure to afford the title compound as a solid.

¹H NMR (500 MHz, acetone-d₆): δ 5.91 (s, 2H), 4.29 (q, 2H), 3.65 (s, 3H), 1.28 (t, 3H). MS (+ESI) m/z 319 (MH⁺).

### Intermediate 17

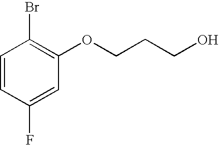

### 3-(2-Bromo-5-fluorophenoxy)propan-1-ol

To a solution of 2-bromo-5-fluorophenol (10.18 g, 53.3 mmol) in DMF (25 mL) cooled at 0° C. was added K₂ CO₃ (8.31 g, 60.1 mmol) and 3-bromopropan-1-ol (6 mL, 66.3 mmol). The reaction mixture was heated to 60° C. for 2 h. The suspension was then poured into aqueous 1 N HCl, extracted with EtOAc and washed with 1 N HCl and brine. The organic layer was dried (Na₂SO₄) and filtered. Solvents were removed under diminished pressure and the resulting crude product was purified by column chromatography on silica gel (gradient 10-50% EtOAc/hexanes) to afford the title compound as a white solid. ¹H NMR (500 MHz, acetone-d₆): δ 7.58 (dd, 1H), 6.97 (dd, 1H), 6.71 (td, 1H), 4.24 (t, 2H), 3.83-3.77 (m, 2H), 3.70 (t, 1H), 2.06-1.99 (m, 2H).

### Intermediate 18

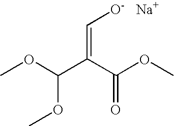

### Sodium 3,3-dimethoxy-2-carbomethoxyprop-1-ene-1-oxide

The intermediate 18 was prepared according to this literature procedure: Zhichkin, P.; Fairfax, D. J.; Eisenbeis, S. A. Synthesis 2002, 6, 720-722.

### Intermediate 19

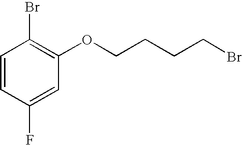

### 1-Bromo-2-(4-bromobutoxy)-4-fluorobenzene

To a solution of 2-bromo-5-fluorophenol (1.3803 g, 7.23 mmol) in DMF (5.09 mL) at 0° C. was added K₂CO₃ (1.11 g, 8.03 mmol) and 1,4-dibromobutane (4.2 mL, 35.5 mmol). The yellow suspension was heated and stirred 1.5 h at 60° C. The reaction mixture was poured into aqueous 1N HCl, extracted with EtOAc and washed with 1N HCl and brine. The organic layer was dried (Na₂SO₄) and filtered. Solvents were removed under diminished pressure to afford the crude product. The crude product was purified by column chromatography on silica gel (40 g) (gradient 0-20% EtOAc/hexanes) to afford the title compound as a colorless oil. ¹H NMR (500 MHz, acetone-d₆): δ 7.61-7.56 (m, 1H), 6.96 (dd, 1H), 6.72 (td, 1H), 4.20 (t, 2H), 3.65 (t, 2H), 2.18-2.10 (m, 2H), 2.06-1.98 (m, 2H).

The following Examples are provided to illustrate the invention and are not to be construed as limiting the scope of the invention in any manner.

### Example 1

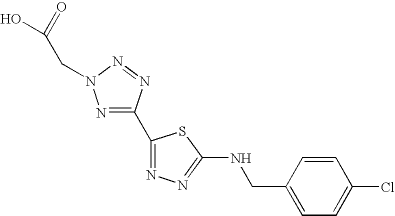

### (5-{5-[(4-Chlorobenzyl)amino]-1,3,4-thiadiazol-2-yl}-2H-tetrazol-2-yl)acetic acid

### Step 1: 5-[(4-Chlorobenzyl)amino]-1,3,4-thiadiazole-2-carbonitrile

To a solution of 4-chlorobenzylamine (3.12 g, 22.1 mmol) in DMF (30 mL) was added K₂ CO₃ (3.66 g, 26.5 mmol) and 5-bromo-1,3,4-thiadiazole-2-carbonitrile (4.2 g, 22.1 mmol). The mixture was stirred at 60° C. for 3 h. The mixture was partitioned between water and ethyl acetate. The water layer was then extracted with ethyl acetate. The combined organic layers were washed with brine, dried over anhydrous Na₂SO₄, filtered, concentrated. The crude product was purified by column chromatography on silica gel to give the title compound. ¹H NMR (400 MHz, CDCl₃): δ 7.29-7.79 (m, 4H), 7.03 (s, 1H), 4.58 (s, 2H). MS: m/z 251 (MH⁺).

### Step 2: N-(4-Chlorobenzyl)-5-(1H-tetrazol-5-yl)-1,3,4-thiadiazol-2-amine

To a suspension of 5-[(4-chlorobenzyl)amino]-1,3,4-thiadiazole-2-carbonitrile (1.0 g, 4 mmol) and ZnBr₂ (0.887 g, 4 mmol) in i-PrOH (10 mL) and H₂O (5 mL) was added NaN₃ (0.52 g, 8 mmol) in a sealed tube. The mixture was stirred at 120° C. overnight. The reaction mixture was cooled to room temperature and then adjusted to pH 4 with 2M aqueous HCl solution. The reaction mixture was extracted with dichloromethane and the combined organic layers were dried over anhydrous Na₂SO₄, filtered and concentrated in vacuum to afford the title compound. ¹H NMR (300 MHz, MeOH-d₄): δ 7.32-7.41 (m, 4H), 4.62 (s, 2H). MS: m/z 294 (MH⁺).

### Step 3: Ethyl(5-{5-[(4-chlorobenzyl)amino]-1,3,4-thiadiazol-2-yl}-2H-tetrazol-2-yl)acetate

To a solution of N-(4-chlorobenzyl)-5-(1H-tetrazol-5-yl)-1,3,4-thiadiazol-2-amine (564 mg, 1.92 mmol) and ethyl bromoacetate (637 mg, 3.84 mmol) in CH₂Cl₂ (20 mL) was added Et₃N (970 mg, 9.6 mmol). The resulting solution was stirred at room temperature overnight. The solvent was removed in vacuum. The residue was partitioned between ethyl acetate and water. The combined organic layers were dried over anhydrous Na₂SO₄, filtered and evaporated in vacuum. The crude product was purified by preparative TLC eluting with petroleum ether:EtOAc (1:1) to afford the title compound. ¹H NMR (300 MHz, CDCl₃): δ 7.32-7.39 (m, 4H), 5.49 (s, 2H), 4.66 (s, 2H), 4.29 (q, J=7 Hz, 2H), 1.30 (t, J=7 Hz, 3H). MS: m/z 380.

### Step 4: (5-{5-[(4-Chlorobenzyl)amino]-1,3,4-thiadiazol-2-yl}-2H-tetrazol-2-yl)acetic acid

To a solution of ethyl(5-{5-[(4-chlorobenzyl)amino]-1,3,4-thiadiazol-2-yl}-2H-tetrazol-2-yl)acetate (179 mg, 0.47 mmol) in EtOH (2 mL) was added 1N aqueous NaOH solution (1.5 mL, 1.5 mmol). The resulted solution was stirred at room temperature overnight.

The solvent was removed in vacuum. The residue was adjusted to pH 1 with N aqueous HCl solution, then extracted with ethyl acetate. The combined organic layers were dried over anhydrous Na₂SO₄, filtered and evaporated in vacuum. The crude product was washed with a mixture of petroleum ether and ethyl acetate to afford the title compound. ¹H NMR (400 MHz, MeOH-d₄): δ 7.40 (d, J=8 Hz, 2H), 7.36 (d, J=8 Hz, 2H), 5.65 (s, 2H), 4.85 (s, 2H). MS: m/z 352 (MH⁺).

### Examples 2 & 3

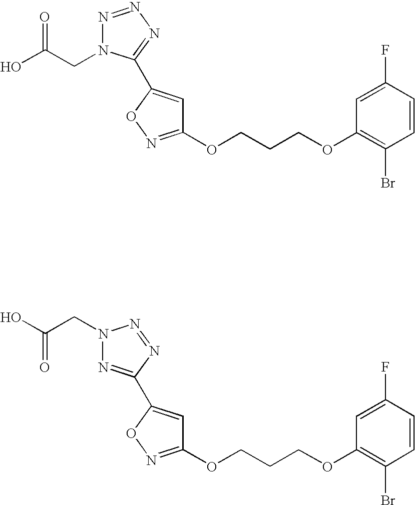

### (5-{3-[3-(2-Bromo-5-fluorophenoxy)propoxy]isoxazol-5-yl}-2H-tetrazol-2-yl)acetic acid (Major Isomer) & (5-{3-[3-(2-bromo-5-fluorophenoxy)propoxy]isoxazol-5-yl}-1H-tetrazol-1-yl)acetic acid (Minor Isomer)

### Step 1: Methyl 3-(3-bromopropoxy)isoxazole-5-carboxylate

To a solution of methyl 3-hydroxyisoxazole-5-carboxylate (1.0150 g, 7.09 mmol) in DMF (5 mL) at 0° C. was added potassium carbonate (1.0872 g, 7.87 mmol) and 1,3-dibromopropane (3.6 mL, 35.5 mmol). The yellow suspension was warmed and heated to 60° C. for 1.5 h. The reaction mixture was poured into aqueous 1 N HCl, extracted with EtOAc and washed with 1 N HCl and brine. The organic layer was dried (Na₂SO₄) and filtered. Solvents were removed under diminished pressure to afford the crude product. The crude product was purified by column chromatography on silica gel (gradient 10-30% EtOAc/hexanes) to afford the title compound as a white solid. ¹H NMR (500 MHz, acetone-d₆): δ 6.81 (s, 1H), 4.45 (t, 2H), 3.95 (s, 3H), 3.67 (t, 2H), 2.39 (p, 2H).

### Step 2: 3-[3-(2-Bromo-5-fluorophenoxy)propox]isoxazole-5-carboxamide

A mixture of 2-bromo-5-fluorophenol (540 mg, 2.83 mmol), methyl 3-(3-bromopropoxy)isoxazole-5-carboxylate (679 mg, 2.57 mmol) and potassium carbonate (426 mg, 3.09 mmol) in DMF (5 ml) was heated at 60° C. for 45 min. The reaction mixture was poured into aqueous 1 N HCl, extracted with EtOAc and washed with 1 N HCl and brine. The organic layer was dried (Na₂SO₄) and filtered. Solvents were removed under diminished pressure to afford the crude product. The crude product was dissolved into THF (15 mL) and treated with ammonia in MeOH (20 mL, 140 mmol) (7.0 M). The reaction mixture was heated in a sealed tube at 125° C. for 30-60 min. After cooling to room temperature, the solvents were evaporated. The crude solid was triturated with ether/hexanes to give the title compound as a white solid. ¹H NMR (400 MHz, acetone-d₆): δ 7.63 (br s, 1H), 7.60 (dd, 1H), 7.21 (br s, 1H), 7.01 (dd, 1H), 6.74 (td, 1H), 6.63 (s, 1H), 4.55 (t, 2H), 4.33 (t, 2H), 2.38 (p, 2H).

### Step 3: 3-[3-(2-Bromo-5-fluorophenoxy)propoxy]isoxazole-5-carbonitrile

A suspension of 3-[3-(2-bromo-5-fluorophenoxy)propoxy]isoxazole-5-carboxamide (701 mg, 1.952 mmol) in CH₂Cl₂ (11 mL) was treated with triethylamine (740 μL, 5.31 mmol), followed by trifluoroacetic anhydride (530 μL, 3.75 mmol) at room temperature. After 30 min, the reaction mixture was poured into aqueous ammonium chloride, extracted with EtOAc and washed with brine. The organic layer was dried (Na₂SO₄) and filtered. Solvents were removed under diminished pressure to afford the crude product. The crude material was purified by column chromatography on silica gel (gradient from 0 to 30% EtOAc/hexanes) to afford the title compound as a colorless oil. ¹H NMR (400 MHz, acetone-d₆): δ 7.61 (dd, 1H), 7.21 (s, 1H), 7.00 (dd, 1H), 6.75 (td, 1H), 4.62 (t, 2H), 4.33 (t, 2H), 2.40 (p, 2H).

### Step 4: 5-{3-[3-(2-Bromo-5-fluorophenoxy)propoxy]isoxazol-5-yl}-1H-tetrazole

A suspension of 3-[3-(2-bromo-5-fluorophenoxy)propoxy]isoxazole-5-carbonitrile (148 mg, 0.434 mmol), sodium azide (373 mg, 5.74 mmol) and pyridine hydrochloride (304 mg, 2.63 mmol) (dried by heating under vacuum) in NMP (3 mL) was heated to 130° C. for 1 h. The reaction mixture was diluted with EtOAc, washed four times with 1 N HCl, washed with brine and dried (Na₂SO₄). The crude material was triturated with ether/hexanes, filtered, and dried to afford the title compound as an off-white solid. ¹H NMR (500 MHz, acetone-d₆): δ 7.16 (dd, 1H), 6.69 (dd, 1H), 6.58 (s, 1H), 6.34 (td, 1H), 4.04 (t, 2H), 3.80 (t, 2H), 1.85-1.80 (m, 2H). MS: m/z 385.9, 383.8 (MH+).

### Step 5: Ethyl(5-{3-[3-(2-bromo-5-fluorophenoxy)propoxy]isoxazol-5-yl}-2H-tetrazol-2-yl)acetate (Major Isomer) & ethyl(5-{3-[3-(2-bromo-5-fluorophenoxy)propoxy]isoxazol-5-yl}-1H-tetrazol-1-yl)acetate (Minor Isomer)

To a solution of 5-{3-[3-(2-bromo-5-fluorophenoxy)propoxy]isoxazol-5-yl}-1H-tetrazole (103 mg, 0.268 mmol) in 1,4-dioxane (2 mL) was added N,N-diisopropylethylamine (140 μL, 0.804 mmol) and ethyl bromoacetate (60 L, 0.539 mmol). The reaction was heated at 90° C. for 1 h in a sealed vial. A white precipitate was filtered off. The reaction mixture was poured into 1N HCl, extracted with EtOAc and washed with brine. The organic layer was dried (Na₂SO₄) and filtered. Solvents were removed under diminished pressure to afford the crude product. The crude material was purified by column chromatography on silica gel (gradient from 0 to 30% EtOAc/hexanes) to afford a mixture of the title compounds as a white solid (regioisomeric ratio 4:1).

Major isomer: ¹H NMR (500 MHz, acetone-d₆): δ 7.76 (dd, 1H), 7.19-7.16 (m, 1H), 7.02 (s, 1H), 6.90 (m, 1H), 6.00 (s, 2H), 4.77 (t, 2H), 4.54-4.44 (m, 4H), 2.57 (m, 2H), 1.46 (t, 3H).

Minor isomer: ¹H NMR (500 MHz, acetone-d₆): δ 7.78-7.74 (m, 1H), 7.19-7.16 (m, 2H), 6.90-6.89 (m, 1H), 5.94 (s, 2H), 4.79-4.75 (m, 2H), 4.54-4.44 (m, 4H), 2.59-2.55 (m, 2H), 1.44-1.41 (m, 3H).

### Step 6: (5-{3-[3-(2-Bromo-5-fluorophenoxy)propoxy]isoxazol-5-yl}-2H-tetrazol-2-yl)acetic acid (Major Isomer) & (5-{3-[3-(2-bromo-5-fluorophenoxy)propoxy]isoxazol-5-yl}-1H-tetrazol-1-yl)acetic acid (Minor Isomer)

To a solution of a mixture of ethyl(5-{3-[3-(2-bromo-5-fluorophenoxy)propoxy]isoxazol-5-yl}-2H-tetrazol-2-yl)acetate (major isomer) & ethyl(5-{3-[3-(2-bromo-5-fluorophenoxy)propoxy]-isoxazol-5-yl}-1H-tetrazol-1-yl)acetate (minor isomer) (ratio 4:1) (64 mg, 0.136 mmol) in THF (4 mL) and MeOH (2 mL) was added 1N aqueous sodium hydroxide (2 mL, 2.0 mmol). After 5 min, the reaction mixture was poured into 1N HCl, extracted with EtOAc and washed with brine. The organic layer was dried (Na₂SO₄) and filtered.

Solvents were removed under diminished pressure to afford the crude product. The crude material was triturated with ether/hexanes to give a mixture of the title compounds as a white solid (regioisomeric ratio 4:1).

Major isomer: ¹H NMR (500 MHz, acetone-d₆): δ 7.60 (dd, 1H), 7.04-7.00 (m, 1H), 6.86 (s, 1H), 6.74 (td, 1H), 5.85 (s, 2H), 4.61 (t, 2H), 4.35 (t, 2H), 2.43-2.38 (m, 2H). MS: m/z 443.8, 441.9 (MH+).

Minor isomer: ¹H NMR (500 MHz, acetone-d₆): δ 7.62-7.58 (m, 1H), 7.04-7.00 (m, 2H), 6.76-6.71 (m, 1H), 5.78 (s, 2H), 4.63-4.59 (m, 2H), 4.37-4.33 (m, 2H), 2.43-2.38 (m, 2H). MS: m/z 443.8, 441.9 (MH+).

### Alternative Method for the Preparation of Example 2

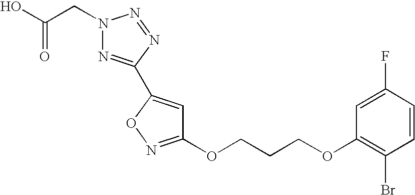

### (5-{3-[3-(2-Bromo-5-fluorophenoxy)propoxy]isoxazol-5-yl}-2H-tetrazol-2-yl)acetic acid

To a solution of 2-bromo-5-fluorophenol (125 mg, 0.654 mmol) in DMF (0.75 mL) was added potassium carbonate (85 mg, 0.615 mmol) and ethyl{5-[3-(3-bromopropoxy) isoxazol-5-yl]-2H-tetrazol-2-yl}acetate (INTERMEDIATE 3) (190 mg, 0.528 mmol) at room temperature. The yellow suspension was heated to 60° C. for 1 h. The reaction mixture was poured into aqueous 1N HCl, extracted with EtOAc and washed with 1N HCl and brine. The organic layer was dried (Na₂SO₄) and filtered. Solvents were removed under diminished pressure to afford the crude product. The crude material was dissolved into MeOH (2 mL) and THF (4 mL) and treated with 1N NaOH (2 mL, 2 mmol). After 5 min, the reaction was poured into aqueous 1N HCl, extracted with EtOAc and washed with 1N HCl and brine. The organic layer was dried (Na₂SO₄) and filtered. Solvents were removed under diminished pressure to afford the crude product. The crude material was triturated twice with ether/heptane to afford the title compound as a white solid.

¹H NMR (400 MHz, acetone-d₆): δ 7.62 (dd, 1H), 7.04 (dd, 1H), 6.88 (s, 1H), 6.76 (td, 1H), 5.87 (s, 2H), 4.64 (t, 2H), 4.38 (t, 2H), 2.46-2.42 (m, 2H). MS: m/z 443.8, 442.0 (MH+).

### Example 4

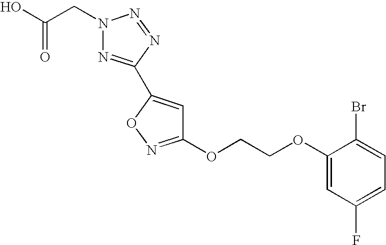

### (5-{3-[2-(2-Bromo-5-fluorophenoxy)ethoxy]isoxazol-5-yl}-2H-tetrazol-2-yl)acetic acid

### Step 1: Methyl 3-[2-(2-bromo-5-fluorophenoxy)ethoxy]isoxazole-5-carboxylate

To a solution of 2-(2-bromo-5-fluorophenoxy)ethanol (510 mg, 2.170 mmol) (INTERMEDIATE 6) and methyl 3-hydroxyisoxazole-5-carboxylate (461 mg, 3.22 mmol) in THF (10 ml) was added di-tert-butyl azodicarboxylate (737 mg, 3.20 mmol). The yellow solution was cooled to −78° C. and treated with a solution of triphenylphosphine (846 mg, 3.23 mmol) in CH₂Cl₂ (5 ml). The final mixture was warmed and stirred for 24 h at room temperature. Solvents were removed under diminished pressure to afford the crude product. The crude material was purified by column chromatography on silica gel (gradient from 0 to 30% EtOAc/hexanes) to afford the title compound as a white solid. ¹H NMR (500 MHz, acetone-d₆): δ 7.61 (dd, 1H), 7.06 (dd, 1H), 6.87 (s, 1H), 6.77 (td, 1H), 4.75-4.72 (m, 2H), 4.56-4.53 (m, 2H), 3.95 (s, 3H).

### Step 2: 3-[2-(2-Bromo-5-fluorophenoxy)ethoxy]isoxazole-5-carboxamide

Methyl 3-[2-(2-bromo-5-fluorophenoxy)ethoxy]isoxazole-5-carboxylate (700 mg, 1.944 mmol) was dissolved into THF (10 mL) and treated with ammonia in MeOH (13.88 mL, 97 mmol) (7.0 M). The reaction mixture was heated in a sealed tube at 125° C. for 30-60 min. The reaction mixture was cooled to room temperature and the solvents were evaporated. The crude material was purified by column chromatography on silica gel by eluting with EtOAc and triturated with ether/hexanes to afford the title compound as a white solid. ¹H NMR (500 MHz, acetone-d₆): δ 7.66 (br s, 1H), 7.61 (dd, 1H), 7.22 (br s, 1H), 7.07 (dd, 1H), 6.77 (td, 1H), 6.68 (s, 1H), 4.73-4.70 (m, 2H), 4.56-4.53 (m, 2H).

### Step 3: 3-[2-(2-Bromo-5-fluorophenoxy)ethoxy]isoxazole-5-carbonitrile

A suspension of 3-[2-(2-bromo-5-fluorophenoxy)ethoxy]isoxazole-5-carboxamide (494 mg, 1.431 mmol) in CH₂Cl₂ (8 mL) was treated with triethylamine (0.5 mL, 3.58 mmol), followed with a solution of trifluoroacetic anhydride (400 L, 2.83 mmol) and triethylamine (0.5 mL, 3.58 mmol) in CH₂Cl₂ (3 mL) at −78° C. The solution was warmed to room temperature. After 45 min, the reaction mixture was poured into aqueous ammonium chloride, extracted with EtOAc and washed with aqueous ammonium chloride and brine. The organic layer was dried (Na₂SO₄) and filtered. Solvents were removed under diminished pressure to afford the crude product. The crude material was purified by column chromatography on silica gel (gradient from 0 to 30% EtOAc/hexanes) to afford the title compound as a white solid. ¹H NMR (400 MHz, acetone-d₆): δ 7.62 (dd, 1H), 7.27 (s, 1H), 7.07 (dd, 1H), 6.78 (td, 1H), 4.82-4.78 (m, 2H), 4.59-4.55 (m, 2H).

### Step 4: 5-{3-[2-(2-Bromo-5-fluorophenoxy)ethoxy]isoxazol-5-yl}-1H-tetrazole

A suspension of 3-[2-(2-bromo-5-fluorophenoxy)ethoxy]isoxazole-5-carbonitrile (315 mg, 0.963 mmol), sodium azide (317 mg, 4.88 mmol) and pyridine hydrochloride (220 mg, 1.904 mmol) (dried by heating under vacuum) in NMP (3 mL) was heated to 140° C. for 30-60 min. The reaction mixture was diluted with EtOAc, washed four times with 1 N HCl, washed with brine and dried (Na₂SO₄). The crude material was triturated with ether/hexanes to afford the title compound as an off-white solid. ¹H NMR (400 MHz, acetone-d₆): δ 7.63 (dd, 1H), 7.09 (dd, 1H), 6.98 (s, 1H), 6.78 (td, 1H), 4.81-4.77 (m, 2H), 4.61-4.57 (m, 2H). MS: m/z 372.0, 369.8 (MH+).

### Step 5: Ethyl(5-{3-[2-(2-bromo-5-fluorophenox)ethoxy]isoxazol-5-yl}-2H-tetrazol-2-yl)acetate and ethyl(5-{3-[2-(2-bromo-5-fluorophenoxy)ethoxy]isoxazol-5-yl}-1H-tetrazol-1-yl)acetate

To a solution of 5-{3-[2-(2-bromo-5-fluorophenoxy)eth oxy]isoxazol-5-yl}-1H-tetrazole (251 mg, 0.678 mmol) in 1,4-dioxane (4 mL) was added N,N-diisopropylethylamine (360 μL, 2.061 mmol) and ethyl bromoacetate (150 μL, 1.347 mmol). The reaction was heated at 90° C. for 1 h in a sealed vial. A white precipitate was filtered off. The reaction mixture was poured into 1N HCl, extracted with EtOAc and washed with brine. The organic layer was dried (Na₂SO₄) and filtered. Solvents were removed under diminished pressure to afford the crude product. The crude material was purified twice by column chromatography on silica gel (gradient from 0 to 50% EtOAc/hexanes) to afford the title compounds.

### Ethyl(5-{3-[2-(2-bromo-5-fluorophenoxy)ethoxy]isoxazol-5-yl}-2H-tetrazol-2-yl)acetate

Major isomer (less polar isomer, R_{f} 0.14 in 80% EtOAc/hexanes) (regioisomeric ratio 43:1). ¹H NMR (400 MHz, acetone-d₆): δ 7.63 (dd, 1H), 7.10 (dd, 1H), 6.93 (s, 1H), 6.78 (td, 1H), 5.86 (s, 2H), 4.81-4.77 (m, 2H), 4.61-4.57 (m, 2H), 4.31 (q, 2H), 1.31 (t, 3H).

### Ethyl(5-{3-[2-(2-bromo-5-fluorophenoxy)ethoxy]isoxazol-5-yl-}-1H-tetrazol-1-yl)acetate

Minor isomer (more polar isomer, R_{f} 0.07 in 80% EtOAc/hexanes) (regioisomeric ratio 3.5:1). ¹H NMR (500 MHz, acetone-d₆): δ 7.61 (dd, 1H), 7.09 (s, 1H), 7.07 (dd, 1H), 6.77 (td, 1H), 5.79 (s, 2H), 4.81-4.77 (m, 2H), 4.60-4.56 (m, 2H), 4.29 (q, 2H), 1.28 (t, 3H).

### Step 6: (5-{3-[2-(2-Bromo-5-fluorophenoxy)ethoxy]isoxazol-5-yl}-2H-tetrazol-2-yl)acetic acid

To a solution of ethyl(5-{3-[2-(2-bromo-5-fluorophenoxy)ethoxy]isoxazol-5-yl}-2H-tetrazol-2-yl)acetate (179 mg, 0.392 mmol) in THF (4 mL) and MeOH (2 mL) was added N aqueous sodium hydroxide (2 mL, 2.0 mmol). After 5 min, the reaction mixture was poured into 1 N HCl, extracted with EtOAc and washed with brine. The organic layer was dried (Na₂SO₄) and filtered. Solvents were removed under diminished pressure to afford the crude product. The crude material was triturated with ether/hexanes, filtered, and dried to afford the title compound as a white solid. ¹H NMR (400 MHz, acetone-d₆): δ 7.62 (dd, 1H), 7.10 (dd, 1H), 6.93 (s, 1H), 6.78 (td, 1H), 5.87 (s, 2H), 4.80-4.77 (m, 2H), 4.60-4.57 (m, 2H).

MS: m/z 429.8, 427.8 (MH+).

### Example 5

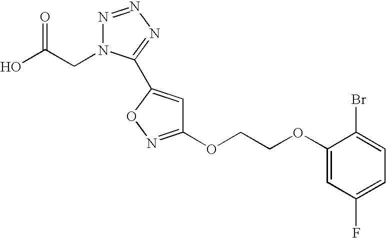

### (5-{3-[2-(2-Bromo-5-fluorophenoxy)ethoxy]isoxazol-5-yl}-1H-tetrazol-1-yl)acetic acid

To a solution of ethyl(5-{3-[2-(2-bromo-5-fluorophenoxy)ethoxy]isoxazol-5-yl}-1H-tetrazol-1-yl)acetate (regioisomers ratio 3.5:1) (59 mg, 0.129 mmol) in THF (4 mL) and MeOH (2 mL) was added 1 N aqueous sodium hydroxide (2 mL, 2.0 mmol). After 5 min, the reaction mixture was poured into 1 N HCl, extracted with EtOAc and washed with brine. The organic layer was dried (Na₂SO₄) and filtered. Solvents were removed under diminished pressure to afford the crude product. The crude material was triturated with ether/hexanes. The suspension was cooled to 0° C. and filtered to give the title compound as a white solid (regioisomeric ratio 4:1 Example 5/Example 4). ¹H NMR (400 MHz, acetone-d₆): δ 7.62 (dd, 1H), 7.10 (s, 1H), 7.09-7.06 (m, 1H), 6.78 (td, 1H), 5.80 (s, 2H), 4.80-4.77 (m, 2H), 4.60-4.57 (m, 2H). MS: m/z 429.8, 427.8 (MH+).

### Example 6

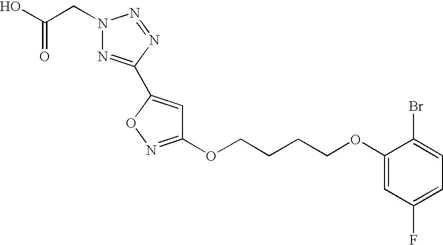

### (5-{3-[4-(2-Bromo-5-fluorophenoxy)butoxy]isoxazol-5-yl}-2H-tetrazol-2-yl)acetic acid

### Step 1: Ethyl(5-{3-[4-(2-bromo-5-fluorophenoxy)butoxy]isoxazol-5-yl}-2H-tetrazol-2-yl)acetate

To a solution of ethyl[5-(3-hydroxyisoxazol-5-yl)-2H-tetrazol-2-yl]acetate (INTERMEDIATE 2) (200 mg, 0.836 mmol) in DMF (0.6 mL) at 0° C. was added potassium carbonate (127 mg, 0.920 mmol) and 1-bromo-2-(4-bromobutoxy)-4-fluorobenzene (INTERMEDIATE 19) (300 mg, 0.920 mmol). The yellow suspension was warmed to room temperature and heated for 1.5 h at 60° C. The reaction mixture was poured into aqueous 1N HCl, extracted with EtOAc and washed with 1N HCl and brine. The organic layer was dried (Na₂SO₄) and filtered. Solvents were removed under diminished pressure to afford the crude product. The crude product was purified by column chromatography on silica gel (40 g) (gradient 10-60% EtOAc/hexanes) followed by trituration with ether/heptane to afford the title compound as a white solid. ¹H NMR (500 MHz, acetone-d₆): δ 7.59 (dd, 1H), 6.98 (dd, 1H), 6.85 (s, 1H), 6.72 (td, 1H), 5.85 (s, 2H), 4.47 (t, 2H), 4.30 (q, 2H), 4.25 (t, 2H), 2.13-2.05 (m, 4H), 1.30 (t, 3H).

### Step 2: (5-{3-[4-(2-Bromo-5-fluorophenoxy)butoxy]isoxazol-5-yl}-2H-tetrazol-2-yl)acetic acid

To a solution of ethyl(5-{3-[4-(2-bromo-5-fluorophenoxy)butoxy]isoxazol-5-yl}-2H-tetrazol-2-yl)acetate (233 mg, 0.481 mmol) in THF (4 mL) and MeOH (2 mL) was added 1N sodium hydroxide (2 mL, 2.0 mmol). After 5 min, the reaction mixture was poured into 1N HCl, extracted with EtOAc and washed with brine. The organic layer was dried (Na₂SO₄) and filtered. Solvents were removed under diminished pressure to afford the crude product. The crude material was triturated with ether/heptane to afford the title compound as a white solid. ¹H NMR (500 MHz, acetone-d₆): δ 7.59 (dd, 1H), 6.98 (dd, 1H), 6.84 (s, 1H), 6.72 (td, 1H), 5.85 (s, 2H), 4.47 (t, 2H), 4.25 (t, 2H), 2.12-2.05 (m, 4H). MS: m/z 456.0, 454.0 (M-H).

### Example 7

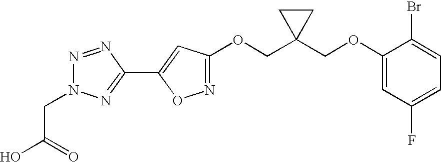

### {5-[3-({1-[(2-Bromo-5-fluorophenoxy)methyl]cyclopropyl}methoxy)isoxazol-5-yl]-2H-tetrazol-2-yl}acetic acid

### Step 1: {1-[(2-Bromo-5-fluorophenoxy)methyl]cyclopropyl}methanol

To a solution of 2-bromo-5-fluorophenol (1.0197 g, 5.34 mmol) and di-tert-butyl azodicarboxylate (1.5115 g, 6.56 mmol) in THF (15 mL) was added 1,1-bis(hydroxymethyl)cyclopropane (2.0175 g, 17.78 mmol). The yellow solution was cooled to −78° C. and treated with a solution of triphenylphosphine (1.6576 g, 6.32 mmol) in CH₂Cl₂ (15 mL). The final mixture was warmed to room temperature and stirred overnight. Solvents were removed under diminished pressure to afford the crude product. The crude material was first purified twice by column chromatography on silica gel (40 g) (gradient from 0 to 30% EtOAc/hexanes). The product was dissolved into heptane and a white solid impurity was removed by filtration. The organic phase was concentrated and purified again by column chromatography on silica gel (120 g) (gradient from 0 to 30% EtOAc/hexanes) to afford the title compound as a colorless oil. ¹H NMR (500 MHz, acetone-d₆): δ 7.58 (dd, 1H), 6.94 (dd, 1H), 6.71 (td, 1H), 4.08 (s, 2H), 3.74 (t, 1H), 3.63 (d, 2H), 0.66-0.57 (m, 4H).

### Step 2: Ethyl{5-[3-({1-[(2-bromo-5-fluorophenoxy)methyl]cyclopropyl}methoxy)isoxazol-5-yl]-2H-tetrazol-2-yl}acetate

To a solution of ethyl[5-(3-hydroxyisoxazol-5-yl)-2H-tetrazol-2-yl]acetate (INTERMEDIATE 2) (200 mg, 0.836 mmol) and {1-[(2-bromo-5-fluorophenoxy)methyl]-cyclopropyl}methanol (345 mg, 1.254 mmol) in THF (3 mL) was added di-tert-butyl azodicarboxylate (289 mg, 1.254 mmol). The yellow solution was cooled to −78° C. and treated with a solution of triphenylphosphine (329 mg, 1.254 mmol) in CH₂Cl₂ (1.5 mL). The final mixture was warmed to room temperature and stirred 24 h. Solvents were removed under diminished pressure to afford the crude product. The crude material was purified twice by column chromatography on silica gel (120 g) (gradient from 0 to 30% EtOAc/hexanes) to afford the title compound as a colorless oil. ¹H NMR (500 MHz, acetone-d₆): δ 7.58 (dd, 1H), 6.97 (dd, 1H), 6.85 (s, 1H), 6.72 (td, 1H), 5.84 (s, 2H), 4.45 (s, 2H), 4.30 (q, 2H), 4.19 (s, 2H), 1.30 (t, 3H), 0.92-0.86 (m, 4H).

### Step 3: {5-[3-({1-[(2-Bromo-5-fluorophenoxy)methyl]cyclopropyl}methoxy)isoxazol-5-yl]-2H-tetrazol-2-yl}acetic acid

To a solution of ethyl{(5-[3-({1-[(2-bromo-5-fluorophenoxy)methyl]cyclopropyl}methoxy)isoxazol-5-yl]-2H-tetrazol-2-yl}acetate (320 mg, 0.645 mmol) in THF (4 mL) and MeOH (2 mL) was added 1N sodium hydroxide (2 mL, 2.0 mmol). After 5 min, the reaction mixture was poured into 1N HCl, extracted with EtOAc and washed with brine. The organic layer was dried (Na₂SO₄) and filtered. Solvents were removed under diminished pressure to afford the crude product. The crude material was triturated with CH₂Cl₂/heptane to afford the title compound as a white solid. ¹H NMR (400 MHz, acetone-d₆): δ 7.59 (dd, 1H), 6.97 (dd, 1H), 6.85 (s, 1H), 6.73 (td, 1H), 5.85 (s, 2H), 4.46 (s, 2H), 4.19 (s, 2H), 0.92-0.89 (m, 4H). MS: m/z 468.0, 465.9 (M-H).

### Example 8

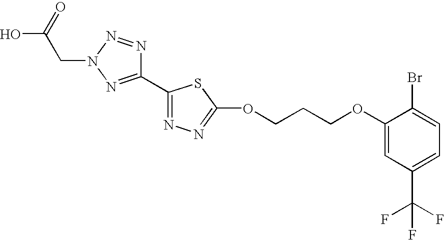

### [5-(5-{3-[2-Bromo-5-(trifluoromethyl)phenoxy]propoxy}-1,3,4-thiadiazol-2-yl)-2H-tetrazol-2-yl]acetic acid

### Step 1: 5-{3-[2-Bromo-5-(trifluoromethyl)phenoxy]propoxy}-1,3,4-thiadiazole-2-carboxamide

To a solution of 3-[2-bromo-5-(trifluoromethyl)phenoxy]propan-1-ol (604 mg, 2.019 mmol) in DMF (6.7 mL) was added sodium hydride (202 mg, 5.05 mmol). After 5 min the 5-bromo-1,3,4-thiadiazole-2-carboxamide (420 mg, 2.019 mmol) was added and the mixture was heated at 60° C. for 0.5 h. The mixture was cooled to room temperature then diluted with water (30 mL). The solid was filtered and washed with water followed by hexanes. The solid was dried under high vacuum to afford the title product. MS: m/z 426, 428 (MH+).

### Step 2: 5-{3-[2-Bromo-5-(trifluoromethyl)phenoxy]propoxy}-1,3,4-thiadiazole-2-carbonitrile

To a solution of 5-{3-[2-bromo-5-(trifluoromethyl)phenoxy]propoxy}-1,3,4-thiadiazole-2-carboxamide and triethylamine (5.7 mL, 4.11 mmol) in THF (5.5 mL) was added TFAA (278 μL, 1.971 mmol) at 0° C. After 5 min the mixture was warmed to room temperature and stirred for a further 0.5 h. The solvent was evaporated and the residue was diluted with water (4 mL). The aqueous layer was extracted with EtOAc (3×4 mL). The combined organic fractions were dried over Na₂SO₄ and the solvent was evaporated. Purification by Combiflash chromatography (SiO₂—12 g, gradient elution of 20-50% EtOAc/hexanes over 25 min) afforded the title product as an oil. MS: m/z 408, 410 (MH+).

### Step 3: 5-(5-{3-[2-Bromo-5-(trifluoromethyl)phenoxy]propoxy}-1,3,4-thiadiazol-2-yl)-1H-tetrazole

A mixture of 5-{3-[2-bromo-5-(trifluoromethyl)phenoxy]propoxy}-1,3,4-thiadiazole-2-carbonitrile (650 mg, 1.592 mmol), sodium azide (155 mg, 2.389 mmol) and ammonium chloride (170 mg, 3.18 mmol) in DMF (4 mL) was heated at 100° C. for 1 h. The mixture was cooled to room temperature, diluted with 1N NaOH (1 mL) and washed with Et₂O (2×2 mL). The aqueous layer was acidified to pH about 1 with 2N HCl and extracted with EtOAc (3×3 mL). The combined organic fractions were washed with water (3 mL) and dried over Na₂SO₄. The solvent was evaporated under reduced pressure to afford the title compound as a solid. MS: m/z 451, 453 (MH+).

### Step 4: Ethyl-[5-(5-{3-[2-bromo-5-(trifluoromethyl)phenoxy]propoxy}-1,3,4-thiadiazol-2-yl)-2H-tetrazol-2-yl]acetate

A mixture of 5-(5-{3-[2-bromo-5-(trifluoromethyl)phenoxy]propoxy}-1,3,4-thiadiazol-2-yl)-1H-tetrazole (310 mg, 0.687 mmol), triethylamine (192 μL, 1.374 mmol), ethyl bromoacetate (115 μL, 1.031 mmol) in THF (3.4 mL) was heated at 80° C. for 1 h. The solvent was evaporated, the residue was diluted with 1N HCl (2 mL) and extracted with EtOAc (3×3 mL). The combined organic fractions were dried over Na₂SO₄. The solvent was evaporated and purification by Combiflash chromatography (SiO₂—12 g, gradient elution of 0-10% EtOAc/CHCl₃ over 25 min) afforded the title compound as the more polar isomer.

¹H NMR (500 MHz, acetone-d₆): δ 7.83 (d, 1H), 7.44 (s, 1H), 7.27 (d, 1H), 5.84 (s, 2H), 4.97-4.88 (m, 2H), 4.49 (t, 2H), 4.31 (q, 2H), 2.55-2.44 (m, 2H), 1.30 (t, 3H). MS: m/z 537, 539 (MH+).

### Step 5: [5-(5-{3-[2-Bromo-5-(trifluoromethyl)phenoxy]propoxy}-1,3,4-thiadiazol-2-yl)-2H-tetrazol-2-yl]acetic acid

To a solution of ethyl-[5-(5-{3-[2-bromo-5-(trifluoromethyl)phenoxy]propoxy}-1,3,4-thiadiazol-2-yl)-2H-tetrazol-2-yl]acetate (55 mg, 0.102 mmol) in THF (341 μL) and MeOH (171 μL) was added 1N NaOH (205 μL, 0.205 mmol) and mixture was stirred at room temperature for 10 min. The THF and MeOH were removed by rotary evaporation and the aqueous layer was washed with Et₂O (2×2 mL). The aqueous layer was acidified to pH 1 with 1N HCl and extracted with EtOAc (3×2 mL). The combined organic fractions were dried over Na₂SO₄ and the solvent was evaporated to afford the title compound as a solid.

¹H NMR (500 MHz, acetone-d₆): δ 7.83 (d, 1H), 7.45 (s, 1H), 7.27 (d, 1H), 5.85 (s, 2H), 4.96-4.90 (m, 2H), 4.49 (t, 2H), 2.55-2.48 (m, 2H). MS: m/z 509, 511 (MH+).

### Example 9

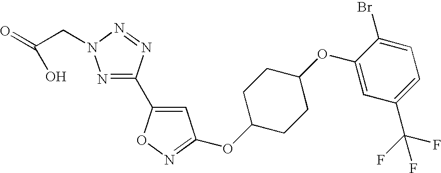

### {5-[3-({4-[2-bromo-5-(trifluoromethyl)phenoxy]cyclohexyl}oxy)isoxazol-5-yl]-2H-tetrazol-2-yl}acetic acid

### Step 1: Ethyl{5-[3-({4-[2-bromo-5-(trifluoromethyl)phenoxy]cyclohexyl}oxy)isoxazol-5-yl]-2H-tetrazol-2-yl}acetate

To a solution of ethyl[5-(3-hydroxyisoxazol-5-yl)-2H-tetrazol-2-yl]acetate (169 mg, 0.708 mmol), 4-[2-bromo-5-(trifluoromethyl)phenoxy]cyclohexanol (200 mg, 0.590 mmol) and triphenylphosphine (186 mg, 0.708 mmol) in THF (5897 μl) was added di-tert-butyl azodicarboxylate (163 mg, 0.708 mmol) at 0° C. The mixture was warmed to room temperature and stirred for 18 h. The solvent was evaporated and the crude product was purified by Combiflash chromatography (SiO₂—40 g, gradient elution of 0-30% EtOAc/hexanes over 25 min) to afford the more polar major isomer as a solid.

¹H NMR (500 MHz, acetone-d₆): δ 7.84 (d, 1H), 7.49 (s, 1H), 7.26 (d, 1H), 6.88 (d, 1H), 5.85 (s, 2H), 4.97-4.90 (m, 2H), 4.30 (q, 2H), 2.35-2.25 (m, 2H), 2.25-2.17 (m, 2H), 1.90 (dd, 4H), 1.35-1.26 (m, 3H). MS: m/z 560, 562 (MH+).

### Step 2: {5-[3-({4-[2-Bromo-5-(trifluoromethyl)phenoxy]cyclohexyl}oxy)isoxazol-5-yl]-2H-tetrazol-2-yl}acetic acid

To a solution of ethyl{5-[3-({4-[2-bromo-5-(trifluoromethyl)phenoxy]cyclohexyl}oxy)isoxazol-5-yl]-2H-tetrazol-2-yl}acetate (205 mg, 0.366 mmol) in THF (1220 μL) and MeOH (610 μL) was added 1N NaOH (732 μL, 0.732 mmol) and the mixture was stirred at room temperature for 10 min. The THF and MeOH were removed by rotary evaporation and the aqueous was layer washed with Et₂O (2×2 mL). The aqueous layer was acidified to pH 1 with 1N HCl and extracted with EtOAc (3×2 mL). The combined organic fractions were dried over Na₂SO₄ and the solvent was evaporated to afford the title compound as a solid.

¹H NMR (500 MHz, acetone-d₆): δ 7.85 (d, 1H), 7.49 (s, 1H), 7.26 (d, 1H), 6.88 (s, 1H), 5.85 (s, 2H), 4.95 (s, 1H), 4.89 (s, 1H), 2.15-1.91 (m, 8H). MS: m/z 532, 534 (MH+).

### Example 10

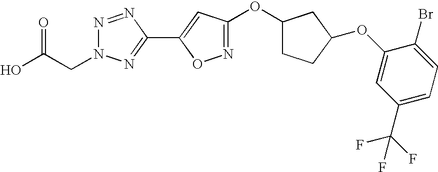

### {5-[3-({3-[2-Bromo-5-(trifluoromethyl)phenoxy]cyclopentyl}oxy)isoxazol-5-yl]-2H-tetrazol-2-yl}acetic acid

### Step 1: Ethyl{5-[3-({3-[2-bromo-5-(trifluoromethyl)phenoxy]cyclopentyl}oxy)isoxazol-5-yl]-2H-tetrazol-2-yl}acetate

The title compound was prepared in a similar manner as that described for Example 9 (step 1) from ethyl{5-[3-({4-[2-bromo-5-(trifluoromethyl)phenoxy]cyclohexyl}oxy)isoxazol-5-yl]-2H-tetrazol-2-yl}acetate, 3-[2-bromo-5-(trifluoromethyl)phenoxy]cyclopentanol, triphenylphosphine and di-tert-butyl azodicarboxylate and isolated as the more polar and major isomer.

¹H NMR (500 MHz, acetone-d₆): δ 7.82 (d, 1H), 7.40 (s, 1H), 7.24 (d, 1H), 6.81 (s, 1H), 5.84 (s, 2H), 5.31-5.23 (m, 2H), 4.30 (q, 2H), 2.35-2.14 (m, 6H), 1.30 (t, 3H). MS: m/z 546, 548 (MH+).

### Step 2: {5-[3-({3-[2-Bromo-5-(trifluoromethyl)phenoxy]cyclopentyl}oxy)isoxazol-5-yl]-2H-tetrazol-2-yl}acetic acid

The title compound was prepared in a similar manner as that described for Example 9 (step 2) from ethyl{5-[3-({3-[2-bromo-5-(trifluoromethyl)phenoxy]-cyclopentyl}oxy)isoxazol-5-yl]-2H-tetrazol-2-yl}acetate and NaOH.

¹H NMR (500 MHz, acetone-d₆): δ 7.82 (d, 1H), 7.40 (s, 1H), 7.24 (d, 1H), 6.81 (s, 1H), 5.84 (s, 2H), 5.31-5.22 (m, 2H), 2.34-2.16 (m, 6H). MS: m/z 518, 520 (MH+).

### Example 11

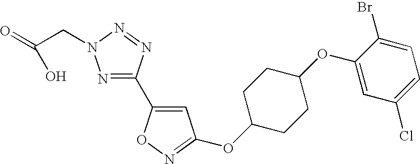

### [5-(3-{[4-(2-Bromo-5-chlorophenoxy)cyclohexyl]oxy}isoxazol-5-yl)-2H-tetrazol-2-yl]acetic acid

### Step 1: Ethyl[5-(3-{[4-(2-bromo-5-chlorophenoxy)cyclohexyl]oxy}isoxazol-5-yl)-2H-tetrazol-2-yl]acetate

The title compound was prepared in a similar manner as that described for Example 9 (step 1) from ethyl{5-[3-({4-[2-bromo-5-(trifluoromethyl)phenoxy]cyclohexyl}oxy)isoxazol-5-yl]-2H-tetrazol-2-yl}acetate, 4-(2-bromo-5-chlorophenoxy)cyclohexanol, triphenylphosphine and di-tert-butyl azodicarboxylate and isolated as the more polar major isomer.

¹H NMR (500 MHz, acetone-d₆): δ 7.59 (dd, 1H), 7.26 (d, 1H), 6.95 (ddd, 1H), 6.88 (s, 1H), 5.85 (s, 2H), 4.91-4.86 (m, 1H), 4.82 (s, 1H), 4.37-4.26 (m, 2H), 2.18-2.06 (m, 6H), 2.00-1.91 (m, 2H), 1.30 (t, 3H). MS: m/z 526, 528 (MH+).

### Step 2 [: 5-(3-{[4-(2-Bromo-5-chlorophenoxy)cyclohexyl]oxy}isoxazol-5-yl)-2H-tetrazol-2-yl]acetic acid

The title compound was prepared in a similar manner as that described for Example 9 (step 2) from ethyl[5-(3-{[4-(2-bromo-5-chlorophenoxy)cyclohexyl]oxy}isoxazol-5-yl)-2H-tetrazol-2-yl]acetate and NaOH.

¹H NMR (500 MHz, acetone-d₆): δ 7.60 (d, 1H), 7.25 (d, 1H), 6.95 (dd, 1H), 6.87 (s, 1H), 5.84 (s, 2H), 4.87 (s, 1H), 4.81 (s, 1H), 2.12-2.04 (m, 6H), 1.97-1.91 (m, 2H). MS: m/z 498, 500 (MH+).

### Example 12

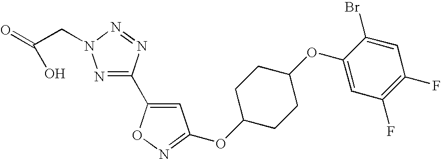

### [5-(3-{[4-(2-Bromo-4,5-difluorophenoxy)cyclohexyl]oxy}isoxazol-5-yl)-2H-tetrazol-2-yl]acetic acid

### Step 1: Ethyl[5-(3-{[4-(2-bromo-4,5-difluorophenoxy)cyclohexyl]oxy}isoxazol-5-yl)-2H-tetrazol-2-yl]acetate

The title compound was prepared in a similar manner as that described for Example 9 (step 1) from ethyl{5-[3-({4-[2-bromo-5-(trifluoromethyl)phenoxy]cyclohexyl}oxy)isoxazol-5-yl]-2H-tetrazol-2-yl}acetate, 4-(2-bromo-4,5-difluorophenoxy)cyclohexanol, triphenylphosphine and di-tert-butyl azodicarboxylate and isolated as the more polar major isomer.

¹H NMR (500 MHz, acetone-d₆): δ 7.52 (dd, 1H), 7.31 (dd, 1H), 6.87 (s, 1H), 5.85 (s, 2H), 4.90 (s, 1H), 4.70 (s, 1H), 4.30 (q, 2H), 2.17-1.94 (m, 8H), 1.30 (t, 3H). MS: m/z 428, 430 (MH+).

### Step 2: [5-(3-{[4-(2-Bromo-4,5-difluorophenoxy)cyclohexyl]oxy}isoxazol-5-yl)-2H-tetrazol-2-yl]acetic acid

The title compound was prepared in a similar manner as that described for Example 9 (step 2) from ethyl[5-(3-{[4-(2-bromo-4,5-difluorophenoxy)cyclohexyl]oxy}isoxazol-5-yl)-2H-tetrazol-2-yl]acetate and NaOH.

¹H NMR (500 MHz, acetone-d₆): δ 7.51 (dd, 1H), 7.29 (dd, 1H), 6.86 (s, 1H), 5.83 (s, 2H), 4.88 (s, 1H), 4.68 (s, 1H), 2.15-1.93 (m, 8H). MS: m/z 500, 502 (MH+).

### Example 13

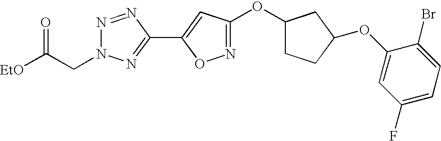

### [5-(3-{[3-(2-Bromo-5-fluorophenoxy)cyclopentyl]oxy}isoxazol-5-yl)-2H-tetrazol-2-yl]acetic acid

### Step 1: Ethyl[5-(3-{[3-(2-bromo-5-fluorophenoxy)cyclopentyl]oxy}isoxazol-5-yl)-2H-tetrazol-2-yl]acetate

The title compound was prepared in a similar manner as that described for Example 9 (step 1) from ethyl{5-[3-({4-[2-bromo-5-(trifluoromethyl)phenoxy]cyclohexyl}oxy)isoxazol-5-yl]-2H-tetrazol-2-yl}acetate, 3-(2-bromo-5-fluorophenoxy)cyclopentanol, triphenylphosphine and di-tert-butyl azodicarboxylate and isolated as the more polar major isomer.

¹H NMR (500 MHz, acetone-d₆): δ 7.61-7.51 (m, 1H), 6.95 (ddd, 1H), 6.82 (d, 1H), 6.71 (td, 1H), 5.84 (s, 2H), 5.26 (s, 1H), 5.08 (s, 1H), 4.30 (q, 2H), 2.73-2.65 (m, 1H), 2.33-2.12 (m, 5H), 1.30 (t, 3H). MS: m/z 496, 498 (MH+).

### Step 2: [5-(3-{[3-(2-Bromo-5-fluorophenoxy)cyclopentyl]oxy}isoxazol-5-yl)-2H-tetrazol-2-yl]acetic acid

The title compound was prepared in a similar manner as that described for Example 9 (step 2) from ethyl[5-(3-{[3-(2-bromo-5-fluorophenoxy)cyclopentyl]oxy}isoxazol-5-yl)-2H-tetrazol-2-yl]acetate and NaOH.

¹H NMR (500 MHz, acetone-d₆): δ 8.03 (s, 1H), 7.43 (d, 1H), 7.26 (s, 1H), 7.16 (s, 1H), 6.29 (s, 2H), 5.71 (s, 1H), 5.54 (s, 1H), 3.14 (d, 1H), 2.79-2.51 (m, 5H). MS: m/z 468, 470 (MH+).

### Example 14

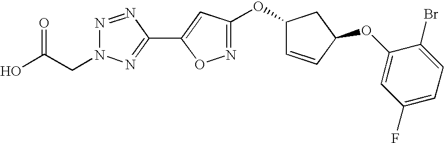

### trans-[5-(3-{[4-(2-Bromo-5-fluorophenoxy)cyclopent-2-en-1-yl]oxy}isoxazol-5-yl)-2H-tetrazol-2-yl]acetic acid

### Step 1: Trans-ethyl[5-(3-{[4-(2-bromo-5-fluorophenoxy)cyclopent-2-en-1-yl]oxy}isoxazol-5-yl)-2H-tetrazol-2-yl]acetate

The title compound was prepared in a similar manner as that described for Example 9 (step 1) from ethyl{5-[3-({4-[2-bromo-5-(trifluoromethyl)phenoxy]cyclohexyl}oxy)isoxazol-5-yl]-2H-tetrazol-2-yl}acetate, cis-4-(2-bromo-5-fluorophenoxy)cyclopent-2-en-1-ol, triphenylphosphine and di-tert-butyl azodicarboxylate.

¹H NMR (500 MHz, acetone-d₆): δ 7.62 (dd, 1H), 7.13 (dd, 1H), 6.87 (s, 1H), 6.76 (td, 1H), 6.51 (d, 2H), 5.97 (d, 1H), 5.85 (s, 2H), 5.79 (d, 1H), 4.31 (q, 2H), 2.71-2.66 (m, 1H), 2.58-2.53 (m, 1H), 1.30 (t, 3H). MS: m/z 505, 507 (MH+).

### Step 2: trans-[[5-(3-{[4-(2-Bromo-5-fluorophenoxy)cyclopent-2-en-1-yl]oxy}isoxazol-5-yl)-2H-tetrazol-2-yl]acetic acid

The title compound was prepared in a similar manner as that described for Example 9 (step 2) from trans-ethyl[5-(3-{[4-(2-bromo-5-fluorophenoxy)cyclopent-2-en-1-yl]oxy}isoxazol-5-yl)-2H-tetrazol-2-yl]acetate and NaOH.

¹H NMR (500 MHz, acetone-d₆): δ 7.62-7.59 (m, 1H), 7.13 (dd, 1H), 6.87 (s, 1H), 6.78-6.75 (m, 1H), 6.50 (s, 2H), 5.97 (d, 1H), 5.85 (s, 2H), 5.79 (s, 1H), 2.71-2.66 (m, 1H), 2.58 (dd, 1H). MS: m/z 466, 468 (MH+).

### Example 15

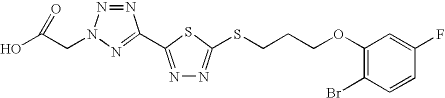

### [5-(5-{[3-(2-Bromo-5-fluorophenoxy)propyl]thio}-1,3,4-thiadiazol-2-yvl)-2H-tetrazol-2-yl]acetic acid

### Step 1: 3-(2-Bromo-5-fluorophenoxy)propane-1-thiol

To a solution of 2-bromo-5-fluorophenol (20 g, 105 mmol) and 1-bromo-3-chloropropane (10.83 mL, 110 mmol) in DMF (200 mL) was added 50% aqueous sodium hydroxide (8.80 g, 110 mmol). The mixture was stirred at 120° C. for 2 d. After cooling, the mixture was diluted with water and extracted with EtOAc. The EtOAc extract was washed with 0.5 M NaOH (2×), brine, dried (Na₂SO₄) and concentrated. Chromatography over silica gel and elution initially with hexanes followed by hexanes:EtOAc (9:1) gave the partially purified 1-bromo-2-(3-chloropropoxy)-4-fluorobenzene intermediate (least polar fraction) as a pale yellow liquid.

To a solution of the 1-bromo-2-(3-chloropropoxy)-4-fluorobenzene (10 g, 37.4 mmol) in DMF (100 mL) was added potassium thioacetate (5.12 g, 44.9 mmol). The mixture was heated at 80° C. bath for 1 h. After cooling, the mixture was diluted with water and extracted with EtOAc. The EtOAc extract was washed with water (3×), brine, dried (Na₂SO₄) and concentrated. Chromatography over silica gel and elution with hexanes:EtOAc (9:1) afforded S-[3-(2-bromo-5-fluorophenoxy)propyl]ethanethioate as light brown liquid. ¹H NMR (400 MHz, acetone-d₆): δ 7.60 (dd, 1H), 6.97 (dd, 1H), 6.74 (td, 1H), 4.20 (t, 2H), 3.13 (t, 2H), 2.35 (s, 3H), 2.17-2.08 (m, 2H).

A solution of S-[3-(2-bromo-5-fluorophenoxy)propyl]ethanethioate (7.6 g, 24.74 mmol) in EtOH (100 mL) was purged with N₂ gas for 15 min. A solution of 5N NaOH (5.94 mL, 29.7 mmol) was added. The mixture was stirred at room temperature for 1 h, diluted with water, acidified with 1N HCl and extracted with EtOAc. The EtOAc extract was washed with diluted brine (2×), dried (Na₂SO₄) and concentrated to give the title compound as a brown liquid. ¹H NMR (500 MHz, CDCl₃): δ 7.49 (dd, 1H), 6.68 (dd, 1H), 6.61 (td, 1H), 4.15 (t, 2H), 2.83 (q, 2H), 2.19-2.12 (m, 2H), 1.45 (t, 1H).

### Step 2: [5-(5-{[3-(2-Bromo-5-fluorophenoxy)propyl]thio}-1,3,4-thiadiazol-2-yl)-2H-tetrazol-2-yl]acetic acid

To a solution of 3-(2-bromo-5-fluorophenoxy)propane-1-thiol (0.7 g, 2.29 mmol) and tert-butyl[5-(5-bromo-1,3,4-thiadiazol-2-yl)-2H-tetrazol-2-yl]acetate (0.79 g, 2.29 mmol) in DMF (15 mL) was added K₂ CO₃ (22 g, 160 mmol). The mixture was stirred at room temperature overnight and then partitioned between EtOAc (10 mL) and water (10 mL). The EtOAc layer was separated and the water layer was extracted with EtOAc (20 mL). The combined organic layers were washed with brine, dried over anhydrous Na₂SO₄, filtered and concentrated. Purification by preparative TLC on silica gel and elution with 1:1 petroleum ether/EtOAc gave tert-butyl[5-(5-{[3-(2-bromo-5-fluorophenoxy)propyl]thio}-1,3,4-thiadiazol-2-yl)-1H-tetrazol-1-yl]acetate and tert-butyl[5-(5-{[3-(2-bromo-5-fluorophenoxy)propyl]thio}-1,3,4-thiadiazol-2-yl)-2H-tetrazol-2-yl]acetate.

The title compound was prepared by treatment of tert-butyl[5-(5-{[3-(2-bromo-5-fluorophenoxy)propyl]thio}-1,3,4-thiadiazol-2-yl)-2H-tetrazol-2-yl]acetate with TFA in CH₂ Cl₂.

¹H NMR (MeOH-d₄, 400 MHz): δ 7.50 (dd, J=6 Hz and 8 Hz, 1H), 6.88 (dd, J=2 Hz and 10 Hz, 1H), 6.62-6.66 (m, 1H), 5.71 (s, 2H), 4.22 (t, J=6 Hz, 2H), 3.67 (t, J=6 Hz, 2H), 2.36-2.42 (m, 2H). MS: m/z 475 (MH⁺).

### Example 16

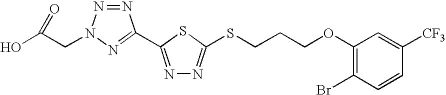

### {5-[5-({3-[2-Bromo-5-(trifluoromethyl)phenoxy]propyl}thio)-1,3,4-thiadiazol-2-yl]-2H-tetrazol-2-yl}acetic acid

The title compound was prepared in a similar manner as that described for Example 15 from 3-[2-bromo-5-(trifluoromethyl)phenoxy]propane-1-thiol and tert-butyl[5-(5-bromo-1,3,4-thiadiazol-2-yl)-2H-tetrazol-2-yl]acetate.

¹HNMR (MeOH-d₆, 400 MHz): δ 7.73 (d, 1H), 7.29 (s, 1H), 7.16 (d, 1H), 5.50 (s, 2H), 4.30 (t, 2H), 3.68 (t, 2H), 2.38-2.45 (m, 2H). MS: m/z 527 (MH⁺).

### Example 17

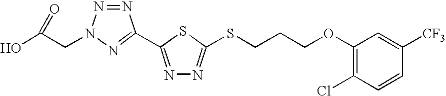

### {5-[5-({3-[2-Chloro-5-(trifluoromethyl)phenoxy]propyl}thio)-1,3,4-thiadiazol-2-yl]-2H-tetrazol-2-yl}acetic acid

The title compound was prepared in a similar manner as described for Example 15 from 3-[2-chloro-5-(trifluoromethyl)phenoxy]propane-1-thiol and tert-butyl[5-(5-bromo-1,3,4-thiadiazol-2-yl)-2H-tetrazol-2-yl]acetate.

¹HNMR (MeOH-d₆, 400 MHz): δ 7.53 (d, 1H), 7.31 (s, 1H), 7.21 (d, 1H), 5.71 (s, 2H), 4.29 (t, 2H), 3.66 (t, 2H), 2.38-2.44 (m, 2H). MS: m/z 481 (MH⁺).

### Example 18

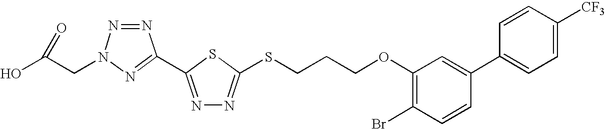

### (5-{5-[(3-{[4-Bromo-4′-(trifluoromethyl)biphenyl-3-yl]oxy}propyl)thio]-1,3,4-thiadiazol-2-yl}-2H-tetrazol-2-yl)acetic acid

The title compound was prepared in a similar manner as that described for Example 15 from 3-{[4-bromo-4′-(trifluoromethyl)biphenyl-3-yl]oxy}propane-1-thiol and tert-butyl [5-(5-bromo-1,3,4-thiadiazol-2-yl)-2H-tetrazol-2-yl]acetate.

¹HNMR (MeOH-d₆, 300 MHz): δ 7.83 (d, 2H), 7.62 (d, 2H), 7.62 (d, 1H), 7.30 (d, 1H), 7.16 (dd, 1H), 5.59 (s, 2H), 4.35 (t, 2H), 3.71 (t, 2H), 2.40-2.48 (m, 2H). MS: m/z 603 (MH⁺).

### Example 19

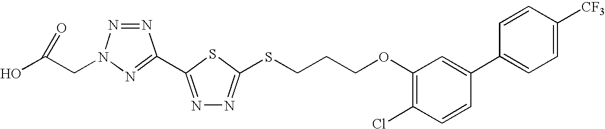

### (5-{5-[(3-{[4-Chloro-4′-(trifluoromethyl)biphenyl-3-yl]oxy}propyl)thiol]-1,3,4-thiadiazol-2-yl}-2H-tetrazol-2-yl)acetic acid

The title compound was prepared in a similar manner as described for Example 15 from 3-{[4-chloro-4′-(trifluoromethyl)biphenyl-3-yl]oxy}propane-1-thiol and tert-butyl[5-(5-bromo-1,3,4-thiadiazol-2-yl)-2H-tetrazol-2-yl]acetate.

¹HNMR (MeOH-d₆, 400 MHz): δ 7.80 (d, 2H), 7.73 (d, 2H), 7.44 (d, 1H), 7.32 (d, 1H), 7.21 (dd, 1H), 5.59 (s, 2H), 4.34 (t, 2H), 3.68 (t, 2H), 2.38-2.45 (m, 2H). MS: m/z 557 (MH⁺).

### Example 20

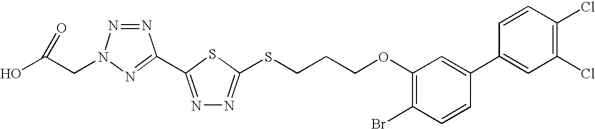

### {5-[5-({3-[(4-Bromo-3′,4′-dichlorobiphenyl-3-yl)oxy]propyl}thio)-1,3,4-thiadiazol-2-yl]-2H-tetrazol-2-yl}acetic acid

The title compound was prepared in a similar manner as that described for Example 15 from 3-[(4-bromo-3′,4′-dichlorobiphenyl-3-yl)oxy]propane-1-thiol and tert-butyl[5-(5-bromo-1,3,4-thiadiazol-2-yl)-2H-tetrazol-2-yl]acetate.

¹HNMR (MeOH-d₆, 300 MHz): δ 7.80 (d, 1H), 7.57-7.63 (m, 3H), 7.24 (d, 1H), 7.08-7.12 (m, 1H), 5.59 (s, 2H), 4.34 (t, 2H), 3.70 (t, 2H), 2.38-2.47 (m, 2H). MS: m/z 603 (MH⁺).

### Example 21

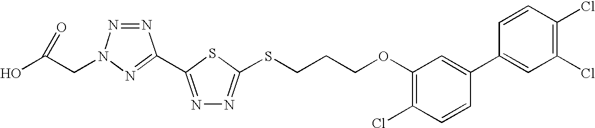

### {5-[5-({3-[(4-Chloro-3′,4′-dichlorobiphenyl-3-yl)oxy]propyl}thio)-1,3,4-thiadiazol-2-yl]-2H-tetrazol-2-yl}acetic acid

The title compound was prepared in a similar manner as described for Example 15 from 3-[(4-chloro-3′,4′-dichlorobiphenyl-3-yl)oxy]propane-1-thiol and tert-butyl[5-(5-bromo-1,3,4-thiadiazol-2-yl)-2H-tetrazol-2-yl]acetate.

¹HNMR (MeOH-d₆, 400 MHz): δ 7.77 (d, 1H), 7.56 (s, 1H), 7.54 (t, 1H), 7.40 (d, 1H), 7.25 (d, 1H), 7.13 (dd, 1H), 5.54 (s, 2H), 4.32 (t, 2H), 3.66 (t, 2H), 2.37-2.43 (m, 2H). MS: m/z: 559 (MH⁺).

### Example 22

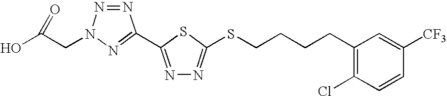

### {5-[5-({4-[2-Chloro-5-(trifluoromethyl)phenyl]butyl}thio)-1,3,4-thiadiazol-2-yl]-2H-tetrazol-2-yl}acetic acid

The title compound was prepared in a similar manner as described for Example 15 from 4-[2-chloro-5-(trifluoromethyl)phenyl]butane-1-thiol and tert-butyl[5-(5-bromo-1,3,4-thiadiazol-2-yl)-2H-tetrazol-2-yl]acetate.

¹HNMR (MeOH-d₆, 400 MHz): δ 7.62 (s, 1H), 7.56 (d, 1H), 7.52 (d, 1H), 5.53 (s, 2H), 3.47 (t, 2H), 2.89 (t, 2H), 1.78-1.97 (m, 4H). MS: m/z 479 (MH⁺).

### Example 23

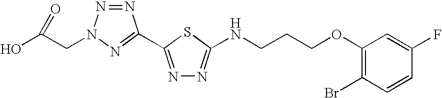

### [5-(5-{[3-(2-Bromo-5-fluorophenoxy)propyl]amino}-1,3,4-thiadiazol-2-yl)-2H-tetrazol-2-yl]acetic acid

### Step 1: 3-(2-Bromo-5-fluorophenoxy)propan-1-amine hydrochloride

A mixture of 2-bromo-5-fluorophenol (30 g, 97 mmol), K₂ CO₃ (20 g, 145 mmol) and N-Boc-3-bromopropylamine (25 g, 106 mmol) in DMF (200 mL) was stirred at 80-100° C. for 2 h. Solvent was removed under vacuum. The residue was diluted with water and extracted Et₂O (3×). The combined organic layers were washed with brine (100 mL), dried over anhydrous Na₂SO₄, filtered, concentrated and purified on silica gel to afford tert-butyl[3-(2-bromo-5-fluorophenoxy)propyl]carbamate. ¹H NMR (CDCl₃ 400 MHz): δ 7.47 (dd, J=6 Hz and 8 Hz, 1H), 6.57-6.68 (m, 2H), 4.07 (t, J=6 Hz, 2H), 3.39 (dd, J=6 Hz and 11 Hz, 2H), 2.02-2.08 (m, 2H), 1.44 (s, 9H).

To a solution of tert-butyl[3-(2-bromo-5-fluorophenoxy)propyl]-carbamate (20 g, 58 mmol) in dioxane (100 mL) was added dropwise a solution of HCl in dioxane (4-5 M, 100 mL). The reaction mixture was stirred at room temperature for 2 h. The solvent was removed under diminised pressure and the residue was washed with Et₂O (100 mL) to afford the title compound as a white solid.

¹HNMR (DMSO-d₆ 300 MHz): δ 8.15 (br, 2H), 7.59 (dd, 1H), 7.06 (dd, 1H), 6.75-6.77 (m, 1H), 4.15 (t, J=6 Hz, 2H), 2.89-3.00 (m, 2H), 2.00-2.09 (m, 2H).

### Step 2: [5-(5-{[3-(2-Bromo-5-fluorophenoxy)propyl]amino}-1,3,4-thiadiazol-2-yl)-2H-tetrazol-2-yl]acetic acid

To a solution of 3-(2-bromo-5-fluorophenoxy)propan-1-amine hydrochloride (1 g, 3.13 mmol) and tert-butyl[5-(5-bromo-1,3,4-thiadiazol-2-yl)-2H-tetrazol-2-yl]acetate (1.08 g, 3.13 mmol) in DMF (20 mL) was added K₂ CO₃ (1.51 g, 11 mmol). The mixture was stirred at room temperature overnight and then partitioned between EtOAc (50 mL) and water (100 mL). The EtOAc layer was separated and the water layer was extracted with EtOAc (50 mL). The combined organic layers were washed with brine (30 mL), dried over anhydrous Na₂SO₄, filtered, and concentrated. Purification by preparative TLC and elution with petroleum ether:EtOAc (1:1) afforded tert-butyl[5-(5-{[3-(2-bromo-5-fluorophenoxy)propyl]amino}-1,3,4-thiadiazol-2-yl)-1H-tetrazol-1-yl]acetate and tert-butyl[5-(5-{[3-(2-bromo-5-fluorophenoxy)propyl]amino}-1,3,4-thiadiazol-2-yl)-2H-tetrazol-2-yl]acetate.

The title compound was prepared by treatment of tert-butyl[5-(5-{[3-(2-bromo-5-fluorophenoxy)propyl]amino}-1,3,4-thiadiazol-2-yl)-2H-tetrazol-2-yl]acetate with TFA in CH₂ Cl₂.

¹HNMR (MeOH-d₄, 300 MHz): δ 7.51 (dd, J=6 Hz and 8 Hz, 1H), 6.88 (dd, J=2 Hz and 10 Hz, 1H), 6.62-6.64 (m, 1H), 5.65 (s, 2H), 4.18 (t, J=6 Hz, 2H), 3.71 (t, J=6 Hz, 2H), 2.20-2.24 (m, 2H). MS: m/z 458 (MH⁺).

### Example 24

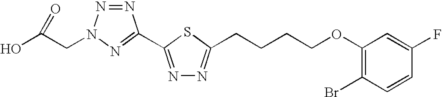

### (5-{5-[4-(2-Bromo-5-fluorophenoxy)butyl]-1,3,4-thiadiazol-2-yl}-2H-tetrazol-2-yl)acetic acid

### Step 1: tert-Butyl {5-[5-(4-hydroxybutyl)-1,3,4-thiadiazol-2-yl]-2H-tetrazol-2-yl}acetate

To a mixture of tert-butyl[5-(5-bromo-1,3,4-thiadiazol-2-yl)-2H-tetrazol-2-yl]acetate (1 g, 2.9 mmol), tert-butyl-but-3-ynyloxy-dimethyl-silane (2.5 g, 36 mmol), PPh₃ (76 mg, 0.29 mmol), Pd(PPh₃)₄ (0.335 g, 0.29 mmol), CuI (55 mg, 0.29 mmol) and Et₃ N (10 mL) in mL of CH₂ Cl₂ was bubbled with Ar gas for 5 min, and then stirred at 60° C. overnight. The mixture was filtered, extracted with CH₂ Cl₂ (50 mL). The combined CH₂ Cl₂ extracts were washed with brine (20 mL), dried over anhydrous Na₂SO₄, concentrated and purified on silica gel to afford tert-butyl {5-[5-(4-{[tert-butyl(dimethyl)silyl]oxy}but-1-yn-1-yl)-1,3,4-thiadiazol-2-yl]-2H-tetrazol-2-yl}acetate.

To a stirred solution of tert-butyl {5-[5-(4-{[tert-butyl(dimethyl)silyl]oxy}but-1-yn-1-yl)-1,3,4-thiadiazol-2-yl]-2H-tetrazol-2-yl}acetate (50 mg, 0.11 mmol) in 5 mL of MeOH was added wet 10% Pd/C (10 mg). The reaction mixture was stirred at room temperature under H₂ atmosphere (18 psi) for 20 h. The mixture was filtered and the filtrate was concentrated. The residue was purified by preparative TLC to afford tert-butyl {5-[5-(4-{[tert-butyl(dimethyl)silyl]oxy}butyl)-1,3,4-thiadiazol-2-yl]-2H-tetrazol-2-yl}acetate.

To a stirred solution of tert-butyl{5-[5-(4-{[tert-butyl(dimethyl)silyl]-oxy}butyl)-1,3,4-thiadiazol-2-yl]-2H-tetrazol-2-yl}acetate (2 g, 4.4 mmol) in 20 mL of THF was added tetrabutylammonium fluoride (3.45 g, 13.4 mmol). The reaction mixture was stirred at room temperature for 4 h. The mixture was partitioned between ethyl acetate (100 mL) and water (100 mL), and the water was extracted with ethyl acetate (100 mL). The combined organic layers were washed with brine, dried over anhydrous Na₂SO₄, concentrated and purified by preparative TLC to afford title compound.

¹HNMR (CDCl₃, 300 MHz): δ 5.42 (s, 2H), 3.72 (d, 1H), 3.27 (d, 1H), 1.94-2.05 (m, 2H), 1.68-1.79 (m, 2H), 1.48 (s, 9H). MS: m/z 268 (MH⁺).

### Step 2: (5-{5-[4-(2-Bromo-5-fluorophenoxy)butyl]-1,3,4-thiadiazol-2-yl}-2H-tetrazol-2-yl)acetic acid

To a solution of tert-butyl {5-[5-(4-hydroxybutyl)-1,3,4-thiadiazol-2-yl]-2H-tetrazol-2-yl}acetate (160 mg, 0.6 mmol), 2-bromo-5-fluorophenol (148 mg, 0.78 mmol) and PPh₃ (204 mg, 0.78 mmol) in CH₂ Cl₂ (10 mL) was added di-isopropyl azodicarboxylate (0.16 mL, 0.78 mmol). After stirring at room temperature for 4 h, the reaction mixture was washed with saturated NaHCO₃ (20 mL) and brine (20 mL). The organic layer was dried over anhydrous sodium sulfate, filtered and evaporated in vacuum. The crude product was purified by preparative TLC to afford tert-butyl (5-{5-[4-(2-bromo-5-fluorophenoxy)butyl]-1,3,4-thiadiazol-2-yl}-2H-tetrazol-2-yl)acetate. Subsquently deprotection with TFA in CH₂ Cl₂ gave the title compound.

¹HNMR (MeOH-d₄, 400 MHz): δ 7.48 (dd, 1H), 6.85 (dd, 1H), 6.59-6.63 (m, 1H), 5.52 (s, 2H), 4.12 (d, 2H), 3.36 (d, 2H), 2.10-2.17 (m, 2H), 1.94-2.01 (m, 2H). MS: m/z 457 (MH⁺).

### Example 25

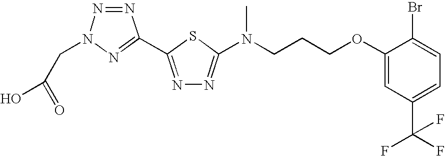

### (5-{5-[{3-[2-Bromo-5-(trifluoromethyl)phenoxy]propyl}(methyl)amino]-1,3,4-thiadiazol-2-yl}-2H-tetrazol-2-yl)acetic acid

### Step 1: tert-Butyl{3-[2-bromo-5-(trifluoromethyl)phenoxy]propyl}carbamate

To a solution of 2-bromo-5-(trifluoromethyl)phenol (3.00 g, 12.46 mmol) and N-(3-hydroxypropyl)carbamic acid tert-butyl ester (3.21 mL, 18.79 mmol) in THF (15 mL) was added di-tert-butyl azodicarboxylate (4.3082 g, 18.71 mmol). The yellow solution was cooled to −78° C. and treated with a solution of triphenylphosphine (4.94 g, 18.83 mmol) in CH₂Cl₂ (15 mL). The final mixture was warmed and stirred 2 h at room temperature. Solvents were removed under diminished pressure to afford the crude product which was purified by column chromatography on silica gel (gradient from 0 to 30% EtOAc/hexanes) to give a colorless oil which solidified as a white solid. ¹H NMR (500 MHz, acetone-d₆): δ 7.83 (d, 1H), 7.37 (s, 1H), 7.25 (d, 1H), 6.16 (br s, 1H), 4.29 (t, 2H), 3.36 (q, 2H), 2.10-2.00 (m, 2H), 1.41 (s, 9H).

### Step 2: 3-[2-Bromo-5-(trifluoromethyl)phenoxy]-N-methylpropan-1-amine

To a stirred solution of tert-butyl {3-[2-bromo-5-(trifluoromethyl)phenoxy]propyl}carbamate (501 mg, 1.258 mmol) in DMF (2 mL) cooled to −78° C. was added 60% NaH in oil (84 mg, 2.10 mmol) and the reaction mixture was allowed to warm to room temperature. After 5 min, the suspension was cooled again to −78° C. and methyl iodide (400 μL, 6.40 mmol) was added. The reaction mixture was warmed and stirred 1 h at room temperature. The reaction was poured into aqueous 1 N HCl, extracted with EtOAc and washed with 1 N HCl and brine.

The organic layer was dried (Na₂SO₄) and filtered. Solvents were removed under diminished pressure to afford the crude tert-butyl carbamate which was dissolved in EtOH (5 mL) and treated with 4 M HCl in dioxane (5 mL) at room temperature. After 3 h, solvents were removed under vacuum. The white solid was poured into aqueous 1 N NaOH, extracted twice with Et₂O and washed with 1 N NaOH and brine. The organic layer was dried (MgSO₄) and filtered. Solvents were removed under diminished pressure to afford the title product as a colorless oil. ¹H NMR (400 MHz, DMSO-d₆): δ 7.83 (d, 1H), 7.40 (s, 1H), 7.25 (d, 1H), 4.22 (t, 2H), 3.33 (br s, 1H), 2.64 (t, 2H), 2.29 (s, 3H), 1.89 (p, 2H).

### Step 3: Ethyl(5-{5-{5-[3-[2-bromo-5-(trifluoromethyl)phenoxy]propyl}(methyl)amino]-1,3,4-thiadiazol-2-yl}-2H-tetrazol-2-yl)acetate

To a solution of 3-[2-bromo-5-(trifluoromethyl)phenoxy]-N-methylpropan-1-amine (105 mg, 0.34 mmol) and Hunig's Base (250 μL, 1.43 mmol) in 1,4-dioxane (1.5 mL) was added ethyl{5-[5-(methylsulfonyl)-1,3,4-thiadiazol-2-yl]-2H-tetrazol-2-yl}acetate (127 mg, 0.40 mmol) (INTERMEDIATE 16). In a sealed vial, the final reaction mixture was heated to 120° C. overnight. Then, the reaction was poured into aqueous NaHCO₃, extracted with EtOAc, washed with brine, dried (Na₂SO₄), filtered and concentrated. The material was purified by column chromatography on silica gel (gradient from 0 to 50% EtOAc/hexanes) to afford the title compound as a waxy oil. ¹H NMR (400 MHz, acetone-d₆): δ 7.81 (d, 1H), 7.37 (s, 1H), 7.22 (d, 1H), 5.76 (s, 2H), 4.37 (t, 2H), 4.27 (q, 2H), 3.90 (t, 2H), 3.31 (s, 3H), 2.34 (p, 2H), 1.27 (t, 3H). MS: m/z=551.9, 549.8 (MH+).

### Step 3: (5-{5-[{3-[2-Bromo-5-(trifluoromethyl)phenoxy]propyl}(methyl)amino]-1,3,4-thiadiazol-2-yl}-2H-tetrazol-2-yl)acetic acid

The ethyl(5-{5-[{3-[2-bromo-5-(trifluoromethyl)phenoxy]propyl}(methyl)amino]-1,3,4-thiadiazol-2-yl}-2H-tetrazol-2-yl)acetate from Step 2 (47 mg, 0.081 mmol) was taken up in MeOH (2 mL) and THF (4 mL) and treated with 1 N NaOH (2 mL). After 15 min, the reaction was poured into aqueous 1 N HCl, extracted with EtOAc and washed with brine.

The organic layer was dried (Na₂SO₄) and filtered. Solvents were removed under diminished pressure and the crude material was triturated with Et₂O/heptane to afford the title compound as a pale yellow solid. ¹H NMR (400 MHz, acetone-d₆): δ 7.84 (d, 1H), 7.41 (s, 1H), 7.26 (d, 1H), 5.79 (s, 2H), 4.40 (t, 2H), 3.94 (t, 2H), 3.34 (s, 3H), 2.41-2.32 (m, 2H). MS: m/z=523.8, 521.9 (MH+).

### Example 26

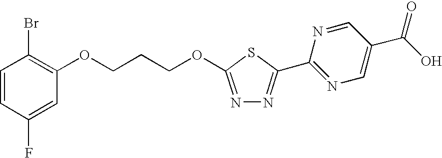

### 2-{5-[3-(2-Bromo-5-fluorophenoxy)propoxy]-1,3,4-thiadiazol-2-yl}pyrimidine-5-carboxylic acid

### Step 1: 5-[3-(2-Bromo-5-fluorophenoxy)propoxy]-1,3,4-thiadiazole-2-carboxamide

To a stirring solution of 3-(2-bromo-5-fluorophenoxy)propan-1-ol (532 mg, 2.136 mmol) (INTERMEDIATE 17) in DMF (5.5 mL) cooled to −78° C. was added 60% NaH in oil (206 mg, 5.15 mmol). The reaction mixture was warmed to room temperature for 5-10 min and cooled again to −78° C. 5-Bromo-1,3,4-thiadiazole-2-carboxamide (406 mg, 1.952 mmol) was then added and the reaction mixture was allowed to warm to room temperature and heated to 60° C. for 1 h. The reaction mixture was cooled to room temperature and poured into 1 N HCl, extracted with EtOAc, washed with brine, dried (Na₂SO₄) and filtered. The organic layer was concentrated to dryness and the residue was triturated with EtOAc/heptane to afford the title compound as a pale yellow solid. ¹H NMR (400 MHz, DMSO-d₆): δ 8.41 (br s, 1H), 8.04 (br s, 1H), 7.62 (dd, 1H), 7.13 (dd, 1H), 6.80 (td, 1H), 4.73 (t, 2H), 4.26 (t, 2H), 2.36-2.28 (m, 2H).

MS: m/z=378.2, 376.0 (MH+).

### Step 2: 5-[3-(2-Bromo-5-fluorophenoxy)propoxy]-1,3,4-thiadiazole-2-carbonitrile

To a suspension of 5-[3-(2-bromo-5-fluorophenoxy)propoxy]-1,3,4-thiadiazole-2-carboxamide (517 mg, 1.37 mmol) and Hunig's Base (2.4 mL, 13.7 mmol) in CH₂Cl₂ (4 mL) was added dropwise trifluoroacetic anhydride (300 μL, 2.12 mmol) at −78° C. The solution was warmed slowly to 0° C. The reaction mixture was then poured into aqueous NH₄Cl, extracted with EtOAc and washed with brine. The organic layer was dried (Na₂SO₄) and filtered. Solvents were removed under diminished pressure and the resulting crude product was purified by column chromatography on silica gel (gradient from 0 to 30% EtOAc/hexanes) to afford the title compound as a solid. ¹H NMR (400 MHz, acetone-d₆): δ 7.61 (dd, 1H), 7.00 (dd, 1H), 6.75 (td, 1H), 4.98 (t, 2H), 4.37 (t, 2H), 2.49 (p, 2H). MS: m/z=359.8, 357.8 (MH+).

### Step 3: Methyl 2-{5-[3-(2-bromo-5-fluorophenoxy)propoxy]-1,3,4-thiadiazol-2-yl}pyrimidine-5-carboxylate

A solution of 5-[3-(2-bromo-5-fluorophenoxy)propoxy]-1,3,4-thiadiazole-2-carbonitrile (235 mg, 0.66 mmol) in DMF (2 mL) was treated with 1.0 M LiHMDS in hexanes (0.722 mL, 0.72 mmol) at −78° C. and warmed to room temperature. After 15 min, NH₄ Cl (108 mg, 2.02 mmol) was added to the reaction mixture followed by sodium 3,3-dimethoxy-2-carbomethoxyprop-1-ene-1-oxide (60% w/w) (380 mg, 1.15 mmol) (INTERMEDIATE 18). The final mixture was heated to 100° C. for 1.5 h. The reaction was then poured into aqueous NH₄Cl, extracted with EtOAc and washed with aqueous NH₄Cl and brine. The organic layer was dried (Na₂SO₄) and filtered. Solvents were removed under diminished pressure and the resulting crude product was purified by column chromatography on silica gel (gradient from 0 to 50% EtOAc/hexanes). The material was triturated with Et₂O/heptane to afford the title compound as a solid. ¹H NMR (400 MHz, acetone-d₆): δ 9.36 (s, 2H), 7.61 (dd, 1H), 7.03 (dd, 1H), 6.75 (td, 1H), 4.93 (t, 2H), 4.39 (t, 2H), 4.02 (s, 3H), 2.49 (p, 2H). MS: m/z=470.8, 468.8 (MH+).

### Step 4: 2-{5-[3-(2-Bromo-5-fluorophenoxy)propoxy]-1,3,4-thiadiazol-2-yl}pyrimidine-5-carboxylic acid

A solution of methyl 2-{5-[3-(2-bromo-5-fluorophenoxy)propoxy]-1,3,4-thiadiazol-2-yl}pyrimidine-5-carboxylate (70 mg, 0.15 mmol) in MeOH (2 mL) and THF (4 mL) was treated with 1N NaOH (2 mL, 2.0 mmol). After 15 min, the reaction was poured into aqueous 1 N HCl, extracted with EtOAc, washed with 1 N HCl, and with brine. The organic layer was dried (Na₂SO₄) and filtered. Solvents were removed under diminished pressure and the product was triturated with Et₂O/heptane to afford the title compound as a solid. ¹H NMR (400 MHz, DMSO-d₆): δ 14.04 (br s, 1H), 9.35 (s, 2H), 7.62 (dd, 1H), 7.15 (dd, 1H), 6.80 (td, 1H), 4.80 (t, 2H), 4.29 (t, 2H), 2.39-2.32 (m, 2H). MS: m/z=454.8, 452.9 (M-H).

### Example 27

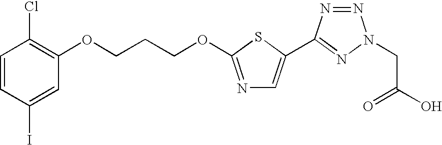

### (5-{2-[3-(2-Chloro-5-iodophenoxy)propoxy]-1,3-thiazol-5-yl}-2H-tetrazol-2-yl)acetic acid

### Step 1: 2-[3-(2-Chloro-5-iodophenoxy)propoxy]-1,3-thiazole-5-carboxamide

To a stirred solution of 3-(2-chloro-5-iodophenoxy)propan-1-ol (1.59 g, 5.09 mmol) in DMF (14 mL) cooled to −78° C. was added 60% NaH in oil (495 mg, 12.38 mmol). The reaction mixture was warmed to room temperature for 5-10 min and cooled again to −78° C. 2-Bromo-1,3-thiazole-5-carboxamide (1.00 g, 4.84 mmol) was then added and the reaction mixture was allowed to warm to room temperature and heated to 60° C. for 45 min. The suspension was cooled to room temperature and poured into 1 N HCl, extracted with EtOAc, washed with 1 N HCl and brine, dried (Na₂SO₄) and filtered. The organic layer was concentrated to dryness to afford a solid which was used in Step 2 without further purification.

### Step 2: 2-[3-(2-Chloro-5-iodophenoxy)propoxy]-1,3-thiazole-5-carbonitrile

To a suspension of 2-[3-(2-chloro-5-iodophenoxy)propoxy]-1,3-thiazole-5-carboxamide (2.37 g, 4.59 mmol) and Hunig's Base (8.0 mL, 45.9 mmol) in CH₂Cl₂ (15 mL) was added dropwise trifluoroacetic anhydride (1.5 mL, 10.62 mmol) at −78° C. The solution was warmed slowly to 0° C. The reaction mixture was then poured into aqueous ammonium chloride, extracted with EtOAc and washed with brine. The organic layer was dried (Na₂SO₄) and filtered. Solvents were removed under diminished pressure to afford a brown oil which was used in Step 3 without further purification.

### Step 3: 5-{2-[3-(2-Chloro-5-iodophenoxy)propoxy]-1,3-thiazol-5-yl}-1H-tetrazole

A suspension of 2-[3-(2-chloro-5-iodophenoxy)propoxy]-1,3-thiazole-5-carbonitrile (2.85 g, 4.40 mmol), NaN₃ (444 mg, 6.83 mmol) and NH₄Cl (752 mg, 14.06 mmol) in DMF (9 mL) was heated to 100° C. for 1.5 h. The reaction mixture was diluted with EtOAc, washed three times with 1 N HCl, brine, dried (Na₂SO₄), filtered and concentrated. The crude material was purified by column chromatography on silica gel (gradient from 0 to 1% AcOH/EtOAc) and then triturated with toluene/heptane to give the title compound as a solid. ¹H NMR (500 MHz, DMSO-d₆): δ 7.88 (s, 1H), 7.50 (s, 1H), 7.32 (d, 1H), 7.22 (d, 1H), 4.66 (t, 2H), 4.26 (t, 2H), 2.31-2.24 (m, 2H).

### Step 4 Ethyl(5-{2-[3-(2-chloro-5-iodophenoxy)propoxy]-1,3-thiazol-5-yl}-2H-tetrazol-2-yl)acetate (Major Isomer) & ethyl(5-{2-[3-(2-chloro-5-iodophenoxy)propoxy]-1,3-thiazol-5-yl}-1H-tetrazol-1-yl)acetate (Minor Isomer)

To a solution of 5-{2-[3-(2-chloro-5-iodophenoxy)propoxy]-1,3-thiazol-5-yl}-1H-tetrazole (1.22 g, 2.62 mmol) in dioxane (15 mL) was added Hunig's Base (1.5 mL, 8.59 mmol) and ethyl bromoacetate (600 μL, 5.39 mmol). The reaction was heated at 90° C. for 1 h. The reaction mixture was poured into 1 N HCl, extracted with EtOAc and washed with brine. The organic layer was dried (Na₂SO₄) and filtered. Solvents were removed under diminished pressure and the crude material was purified by column chromatography on silica gel (gradient from 0 to 20% EtOAc/CHCl₃) to give the title compounds.

Major isomer: Rf: 0.6 with 10% EtOAc/CHCl₃.

Minor isomer: Rf: 0.4 with 10% EtOAc/CHCl₃.

### Step 5: (5-{2-[3-(2-Chloro-5-iodophenoxy)propoxy]-1,3-thiazol-5-yl}-2H-tetrazol-2-yl)acetic acid

Ethyl(5-{2-[3-(2-chloro-5-iodophenoxy)propoxy]-1,3-thiazol-5-yl}-2H-tetrazol-2-yl)acetate (major isomer: Rf: 0.6 with 10% EtOAc/CHCl₃) from Step 4 was taken up in MeOH:THF (1:2) and treated with 1 N NaOH. After 15 min, the reaction was poured into aqueous 1 N HCl, extracted with EtOAc and washed with brine. The organic layer was dried (Na₂SO₄) and filtered. Solvents were removed under diminished pressure and the crude material was triturated with Et₂O/heptane to afford the title compound as a solid. ¹H NMR (500 MHz, DMSO-d₆): δ 13.82 (br s, 1H), 7.93 (s, 1H), 7.50 (s, 1H), 7.32 (d, 1H), 7.22 (d, 1H), 5.75 (s, 2H), 4.66 (t, 2H), 4.26 (t, 2H), 2.32-2.24 (m, 2H). MS: m/z=521.8, 519.7 (M-H).

### Example 28

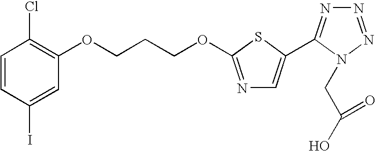

### (5-{2-[3-(2-Chloro-5-iodophenoxy)propoxy]-1,3-thiazol-5-yl}-1H-tetrazol-1-yl)acetic acid

Ethyl(5-{2-[3-(2-chloro-5-iodophenoxy)propoxy]-1,3-thiazol-5-yl}-1H-tetrazol-1-yl)acetate (minor isomer Rf: 0.4 with 10% EtOAc/CHCl₃) prepared in EXAMPLE 27, step 4 was taken up in MeOH:THF (1:2) and treated with 1 N NaOH. After 15 min, the reaction was poured into aqueous 1 N HCl, extracted with EtOAc and washed with brine. The organic layer was dried (Na₂SO₄) and filtered. Solvents were removed under diminished pressure and the crude material was triturated with Et₂O/heptane to afford the title compound as a solid. ¹H NMR (500 MHz, DMSO-d₆): δ 13.93 (br s, 1H), 7.85 (s, 1H), 7.50 (s, 1H), 7.33-7.30 (m, 1H), 7.22 (d, 1H), 5.64 (s, 2H), 4.66 (t, 2H), 4.26 (t, 2H), 2.32-2.24 (m, 2H). MS: m/z=521.8, 519.8 (M-H).

### Example 29

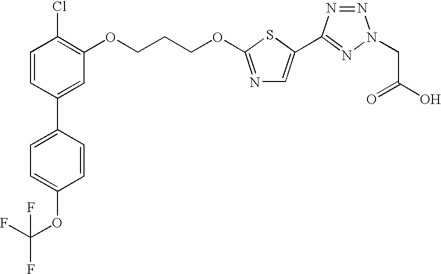

### {5-[2-(3-({[4-Chloro-4′-(trifluoromethoxy)biphenyl-3-yl]oxy}propoxy)-1,3-thiazol-5-yl]-2H-tetrazol-2-yl}acetic acid

To a solution of (5-{2-[3-(2-chloro-5-iodophenoxy)propoxy]-1,3-thiazol-5-yl}-2H-tetrazol-2-yl)acetic acid (208 mg, 0.399 mmol) (EXAMPLE 27) and [4-(trifluoromethoxy)phenyl]boronic acid (128 mg, 0.622 mmol) in toluene (6 mL) was added aqueous 2 M Na₂CO₃ (2 mL, 4.0 mmol), and Pd(Ph₃P)₄ (33 mg, 0.029 mmol). After the resulting heterogeneous mixture was purged with nitrogen, it was gently heated to 80° C. overnight with stirring under a nitrogen atmosphere. After cooling to room temperature, the reaction was poured into a mixture of water and Et₂O, extracted three times with 1 N NaOH, and the aqueous layer was washed again with Et₂O. The aqueous phase and the white suspension were neutralized with 4 N HCl, and extracted with EtOAc. The organic layer was washed with brine and dried (Na₂SO₄). Solvents were removed under reduced pressure and the crude material was purified by column chromatography on silica gel (CH₂ Cl₂/EtOH/H₂O/AcOH: from 98/2/0/0 to 80/20/2/1, and to 70/30/3/1). After concentration, the white solid was diluted with EtOAc and washed with 1 N HCl and brine, dried (Na₂SO₄), filtered and concentrated. The resulting solid was triturated with Et₂O/heptane to afford the title compound as a solid. ¹H NMR (400 MHz, DMSO-d₆): δ 13.82 (br s, 1H), 7.94 (s, 1H), 7.85 (d, 2H), 7.54 (d, 1H), 7.49-7.45 (m, 3H), 7.28 (dd, 1H), 5.74 (s, 2H), 4.71 (t, 2H), 4.39 (t, 2H), 2.36-2.31 (m, 2H). MS: m/z=556.0, 554.0 (M-H).

### Example 30

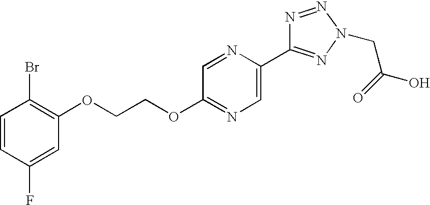

### (5-{5-[2-(2-Bromo-5-fluorophenoxy)ethoxy]pyrazin-2-yl}-2H-tetrazol-2-yl)acetic acid

### Step 1: Methyl 5-[2-(2-bromo-5-fluorophenoxy)ethoxy]pyrazine-2-carboxylate

To a stirring solution of 2-(2-bromo-5-fluorophenoxy)ethanol (2.00 g, 8.49 mmol) (INTERMEDIATE 6) in DMF (10 mL) cooled to −78° C., was added 60% NaH in oil (358 mg, 8.95 mmol). The reaction mixture was warmed to room temperature for 20-30 min and cooled again to −78° C. Methyl 5-chloropyrazine-2-carboxylate (1.02 g, 5.88 mmol) was then added and the reaction mixture was allowed to warm to room temperature. After 1 h at room temperature, the reaction mixture was poured into 1 N HCl, extracted with EtOAc, washed twice with 1 N HCl and brine, dried (Na₂SO₄) and filtered. The organic layer was concentrated to dryness. The crude material was subjected to column chromatography on silica gel (gradient from 0 to 50% EtOAc/hexanes). The resulting product was used in Step 2 without further purification.

### Step 2: 5-[2-(2-Bromo-5-fluorophenoxy)ethoxy]pyrazine-2-carboxamide

In a sealed tube, a solution of methyl 5-[2-(2-bromo-5-fluorophenoxy)ethoxy]pyrazine-2-carboxylate in THF (10 mL) was treated with ammonia in MeOH (7.0 M) (10 mL, 70.0 mmol) and the reaction mixture was heated to 125° C. for 5 h. Solvents were removed under diminished pressure and the resulting crude material was recrystallized from EtOAc/heptane to afford a 1:1 mixture of the title compound and 5-methoxypyrazine-2-carboxamide. This material was used in Step 3 without further purification.

### Step 3: 5-[2-(2-Bromo-5-fluorophenoxy)ethoxy]pyrazine-2-carbonitrile

To a suspension of 5-[2-(2-bromo-5-fluorophenoxy)ethoxy]pyrazine-2-carboxamide from Step 2 (856 mg, 1.68 mmol) and Hunig's Base (3 mL, 17.2 mmol) in CH₂Cl₂ (10 mL) was added dropwise trifluoroacetic anhydride (750 L, 5.31 mmol) at −78° C. The solution was warmed slowly to 0° C. The reaction mixture was then poured into aqueous NH₄Cl, extracted with EtOAc and washed with brine. The organic layer was dried (Na₂SO₄) and filtered. Solvents were removed under diminished pressure and the resulting crude material was purified by column chromatography on silica gel (gradient from 0 to 30% EtOAc/hexanes) to afford the title compound as a solid. ¹H NMR (400 MHz, acetone-d₆): δ 8.76 (d, 1H), 8.44 (d, 1H), 7.61 (dd, 1H), 7.07 (dd, 1H), 6.77 (td, 1H), 4.96-4.93 (m, 2H), 4.61-4.57 (m, 2H). MS: m/z=340.0, 338.0 (MH+).

### Step 4: 2-[2-(2-Bromo-5-fluorophenoxy)ethoxy]-5-(1H-tetrazol-5-yl)pyrazine

A stirred suspension of 5-[2-(2-bromo-5-fluorophenoxy)ethoxy]pyrazine-2-carbonitrile (300 mg, 0.89 mmol), NH₄Cl (150 mg, 2.80 mmol) and NaN₃ (100 mg, 1.54 mmol) in DMF (2 mL) was heated to 100° C. for 2 h. The reaction mixture was diluted with EtOAc, washed three times with 1 N HCl, brine, dried (Na₂SO₄), filtered and concentrated. The crude material was triturated with Et₂O/hexanes to give the title compound as a solid. ¹H NMR (400 MHz, DMSO-d₆): δ 9.01 (d, 1H), 8.61 (d, 1H), 7.62 (dd, 1H), 7.21 (dd, 1H), 6.82 (td, 1H), 4.85-4.80 (m, 2H), 4.54-4.49 (m, 2H). MS: m/z=381.0, 379.0 (M-H).

### Step 5: Ethyl(5-{5-[2-(2-bromo-5-fluorophenoxy)ethoxy]pyrazin-2-yl}-2H-tetrazol-2-yl)acetate (major isomer) & ethyl(5-{5-[2-(2-bromo-5-fluorophenoxy)ethoxy]pyrazin-2-yl}-1H-tetrazol-1-yl)acetate (Minor Isomer)

To a solution of 2-[2-(2-bromo-5-fluorophenoxy)ethoxy]-5-(1H-tetrazol-5-yl)pyrazine (271 mg, 0.71 mmol) in dioxane (4 mL) was added Hunig's Base (370 μL, 2.12 mmol) and ethyl bromoacetate (160 μL, 1.44 mmol). The reaction was heated at 90° C. for 1 h.

The reaction mixture was poured into 1 N HCl, extracted with EtOAc and washed with brine.

The organic layer was dried (Na₂SO₄) and filtered. Solvents were removed under diminished pressure and the crude material obtained was purified by column chromatography on silica gel (gradient from 0 to 30% EtOAc/toluene) to give the two title compounds as regioisomers.

Major isomer: Rf: 0.4 with 20% EtOAc/toluene.

Minor isomer: Rf: 0.6 with 20% EtOAc/toluene.

### Step 6: (5-{5-[2-(2-Bromo-5-fluorophenoxy)ethoxy]pyrazin-2-yl}-2H-tetrazol-2-yl)acetic acid

Ethyl(5-{5-[2-(2-bromo-5-fluorophenoxy)ethoxy]pyrazin-2-yl}-2H-tetrazol-2-yl)acetate (major isomer: Rf: 0.4 with 20% EtOAc/toluene) from Step 5 was taken up in MeOH:THF (1:2) and treated with 1 N NaOH. After 15 min, the reaction was poured into aqueous 1 N HCl, extracted with EtOAc and washed with brine. The organic layer was dried (Na₂SO₄) and filtered. Solvents were removed under diminished pressure and the crude material was triturated with Et₂O/heptane to afford the title compound as a solid. ¹H NMR (400 MHz, DMSO-d₆): δ 13.82 (br s, 1H), 8.95 (d, 1H), 8.55 (d, 1H), 7.62 (dd, 1H), 7.21 (dd, 1H), 6.82 (td, 1H), 5.81 (s, 2H), 4.84-4.79 (m, 2H), 4.54-4.49 (m, 2H). MS: m/z=438.8, 436.8 (M-H).

### Example 31

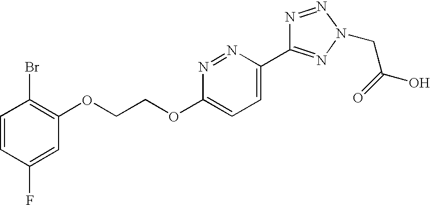

### (5-{6-[2-(2-Bromo-5-fluorophenoxy)ethoxy]pyridazin-3-yl}-2H-tetrazol-2-yl)acetic acid

### Step 1: 3-[2-(2-Bromo-5-fluorophenoxy)ethoxy]-6-(1H-tetrazol-5-yl)pyridazine

To a stirred solution of 2-(2-bromo-5-fluorophenoxy)ethanol (497 mg, 2.11 mmol) (INTERMEDIATE 6) in DMF (3 mL) cooled to −78° C. was added 60% NaH in oil (92 mg, 2.30 mmol). The reaction mixture was warmed to room temperature for 5-10 min and cooled again to −78° C. 6-Chloropyridazine-3-carbonitrile (252 mg, 1.81 mmol) was then added and the reaction mixture was allowed to warm to room temperature. After 1 h at room temperature, NH₄ Cl (332 mg, 6.21 mmol) and NaN₃ (206 mg, 3.17 mmol) were added and the final reaction mixture was heated to 100° C. for 2 h. The reaction mixture was diluted with EtOAc, washed three times with 1 N HCl, brine, dried (Na₂SO₄), filtered and concentrated. The crude material was triturated with EtOAc/hexanes to afford the title compound as a solid. ¹H NMR (400 MHz, DMSO-d₆): δ 8.36 (d, 1H), 7.62 (dd, 1H), 7.57 (d, 1H), 7.22 (dd, 1H), 6.82 (td, 1H), 4.97-4.93 (m, 2H), 4.58-4.54 (m, 2H). MS: m/z=380.8, 379.0 (M-H).

### Step 2: Ethyl(5-{6-[2-(2-bromo-5-fluorophenoxy)ethoxy]pyridazin-3-yl}-2H-tetrazol-2-yl)acetate (major isomer) & ethyl(5-{6-[2-(2-bromo-5-fluorophenoxy)ethoxy]pyridazin-3-yl}-1H-tetrazol-1-yl)acetate (Minor Isomer)

To a solution of 3-[2-(2-bromo-5-fluorophenoxy)ethoxy]-6-(1H-tetrazol-5-yl)pyridazine (460 mg, 1.21 mmol) in dioxane (8 mL) was added Hunig's Base (650 μL, 3.72 mmol) and ethyl bromoacetate (270 μL, 2.43 mmol). The reaction was heated at 90° C. for 1 h. The reaction mixture was poured into 1 N HCl, extracted with EtOAc and washed with brine. The organic layer was dried (Na₂SO₄) and filtered. Solvents were removed under diminished pressure and the crude material obtained was purified by column chromatography on silica gel (gradient from 0 to 30% EtOAc/CHCl₃) to give the two title compounds.

Major isomer: Rf: 0.2 with 10% EtOAc/CHCl₃.

Minor isomer: Rf: 0.4 with 10% EtOAc/CHCl₃.

Step 3: (5-{6-[2-(2-Bromo-5-fluorophenoxy)ethoxy]pyridazin-3-yl}-2H-tetrazol-2-yl)acetic acid

Ethyl(5-{6-[2-(2-bromo-5-fluorophenoxy)ethoxy]pyridazin-3-yl}-2H-tetrazol-2-yl)acetate (major isomer: Rf: 0.2 with 10% EtOAc/CHCl₃) from step 2 was taken up in MeOH:THF (1:2) and treated with 1 N NaOH. After 15 min, the reaction was poured into aqueous 1 N HCl, extracted with EtOAc and washed with brine. The organic layer was dried (Na₂SO₄) and filtered. Solvents were removed under diminished pressure and the crude material was triturated with EtOAc/heptane to afford the title compound as a solid. ¹H NMR (400 MHz, DMSO-d₆): δ 13.86 (br s, 1H), 8.30 (d, 1H), 7.63 (dd, 1H), 7.52 (d, 1H), 7.23 (dd, 1H), 6.82 (td, 1H), 5.86 (s, 2H), 4.96-4.92 (m, 2H), 4.58-4.53 (m, 2H). MS: m/z=439.0, 437.0 (M-H).

### Example 32

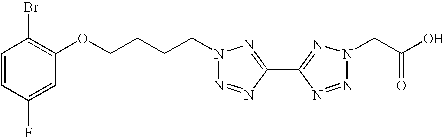

### {2′-[4-(2-Bromo-5-fluorophenoxy)butyl]-2H,2′H-5,5′-bitetrazol-2-yl}acetic acid

### Step 1: Ethyl 2-[4-(2-bromo-5-fluorophenoxy)butyl]-2H-tetrazole-5-carboxylate (Major Isomer) & ethyl 1-[4-(2-bromo-5-fluorophenoxy)butyl]-1H-tetrazole-5-carboxylate (Minor Isomer)

To a solution of 1-bromo-2-(4-bromobutoxy)-4-fluorobenzene (INTERMEDIATE 19) (1.01 g, 3.10 mmol) in DMF (2 mL) was added 1H-tetrazole-5-carboxylic acid ethyl ester sodium salt (758 mg, 4.62 mmol) at room temperature and the final suspension was heated to 60° C. for 2 h. The reaction mixture was then poured into 1 N HCl, extracted with EtOAc, washed with brine, dried (Na₂SO₄) and filtered. Solvents were removed under diminished pressure and the crude material obtained was purified by column chromatography on silica gel (gradient from 0 to 20% EtOAc/toluene) to give the two title regioisomers as colorless oils.

Major isomer (regioisomeric ratio 15:1): Rf: 0.7 with 20% EtOAc/toluene. ¹H NMR (400 MHz, DMSO-d₆): δ 7.61 (dd, 1H), 7.07 (dd, 1H), 6.78 (td, 1H), 4.92 (t, 2H), 4.43 (q, 2H), 4.13 (t, 2H), 2.21-2.12 (m, 2H), 1.86-1.74 (m, 2H), 1.36 (t, 3H). MS: m/z=389.0, 387.0 (MH+).

Minor isomer (regioisomeric ratio 7:1): Rf: 0.6 with 20% EtOAc/toluene. ¹H NMR (400 MHz, DMSO-d₆): δ 7.61 (dd, 1H), 7.08 (dd, 1H), 6.78 (td, 1H), 4.79 (t, 2H), 4.45 (q, 2H), 4.12 (t, 2H), 2.14-2.04 (m, 2H), 1.85-1.75 (m, 2H), 1.37 (t, 3H). MS: m/z=389.0, 387.0 (MH+).

### Step 2: 2-[4-(2-Bromo-5-fluorophenoxy)butyl]-2H-tetrazole-5-carboxamide

A solution of ethyl 2-[4-(2-bromo-5-fluorophenoxy)butyl]-2H-tetrazole-5-carboxylate (major isomer: Rf: 0.7 with 20% EtOAc/toluene) (547 mg, 1.413 mmol) in THF (6 mL) was treated with NH₃ in MeOH (7.0 M) (10 mL, 70.0 mmol). The reaction mixture was heated in a sealed tube at 125° C. for 1 h. The solvents were then evaporated under diminished pressure and the resulting crude material was triturated with ether/hexanes to afford the title compound as a solid (regioisomeric ratio 16:1). ¹H NMR (400 MHz, DMSO-d₆): δ 8.44 (br s, 1H), 8.11 (br s, 1H), 7.72 (dd, 1H), 7.19 (dd, 1H), 6.89 (td, 1H), 4.98 (t, 2H), 4.24 (t, 2H), 2.32-2.20 (m, 2H), 1.96-1.85 (m, 2H). MS: m/z=381.9, 380.0 (M+Na), 360.0, 358.0 (MH+).

### Step 3: 2-[4-(2-Bromo-5-fluorophenoxy)butyl]-2H-tetrazole-5-carbonitrile

To a suspension of 2-[4-(2-bromo-5-fluorophenoxy)butyl]-2H-tetrazole-5-carboxamide (450 mg, 1.26 mmol) and Hunig's Base (2.2 mL, 12.6 mmol) in CH₂ Cl₂ (4 mL) was added dropwise trifluoroacetic anhydride (270 L, 1.91 mmol) at −78° C. The suspension was warmed and stirred at room temperature for 1 h. An additional amount of trifluoroacetic anhydride (130 μL, 0.92 mmol) was added at room temperature. After 30 min, the reaction mixture was diluted with EtOAc and poured into aqueous NH₄ Cl, extracted with EtOAc and washed with brine. The organic layer was dried (Na₂SO₄) and filtered. Solvents were removed under diminished pressure and the resulting crude product was purified by column chromatography on silica gel (gradient from 0 to 30% EtOAc/Hexanes) to afford the title compound as a yellow oil (regioisomeric ratio 9:1). ¹H NMR (400 MHz, acetone-d₆): δ 7.76 (dd, 1H), 7.12 (dd, 1H), 6.90 (td, 1H), 5.24 (t, 2H), 4.40 (t, 2H), 2.60-2.47 (m, 2H), 2.22-2.12 (m, 2H).

### Step 4: 2′-[4-(2-Bromo-5-fluorophenoxy)butyl]-1H,2′H-5,5′-bitetrazole

A suspension of 2-[4-(2-bromo-5-fluorophenoxy)butyl]-2H-tetrazole-5-carbonitrile (200 mg, 0.53 mmol), NaN₃ (171 mg, 2.63 mmol) and pyridinium hydrochloride (125 mg, 1.08 mmol) (dried by heating under high vacuum) in NMP (1.5 mL) was heated to 150° C. for 5 h. The reaction mixture was diluted with EtOAc, washed four times with 1 N HCl, brine and dried (Na₂SO₄). The organic phase was treated with active charcoal and filtered through a pad of Celite. Solvents were removed under diminished pressure to give the title compound as a brown oil. ¹H NMR (400 MHz, DMSO-d₆): δ 7.57 (dd, 1H), 7.06 (dd, 1H), 6.75 (td, 1H), 4.95 (t, 2H), 4.12 (t, 2H), 2.24-2.13 (m, 2H), 1.85-1.76 (m, 2H). MS: m/z=383.0, 381.0 (M-H).

### Step 5: Ethyl{2′-[4-(2-bromo-5-fluorophenoxy)butyl]-2H,2′H-5,5′-bitetrazol-2-yl}acetate (Major Isomer) & ethyl{2′-[4-(2-bromo-5-fluorophenoxy)butyl]-1H,2′H-5,5′-bitetrazol-1-yl}acetate (Minor Isomer)

To a solution of 2′-[4-(2-bromo-5-fluorophenoxy)butyl]-1H,2′H-5,5′-bitetrazole (149 mg, 0.389 mmol) in dioxane (3 mL) was added Hunig's Base (210 μL, 1.20 mmol) and ethyl bromoacetate (100 μL, 0.90 mmol). The reaction was heated at 90° C. for 1.5 h. The reaction mixture was poured into 1 N HCl, extracted with EtOAc and washed with brine. The organic layer was dried (Na₂SO₄) and filtered. Solvents were removed under diminished pressure and the resulting crude material was purified by column chromatography on silica gel (gradient from 0 to 20% EtOAc/CHCl₃) to give the two title regioisomers.

Major isomer: Rf: 0.5 with 10% EtOAc/CHCl₃.

Minor isomer: Rf: 0.4 with 10% EtOAc/CHCl₃.

### Step 6: {2′-[4-(2-Bromo-5-fluorophenoxy)butyl]-2H,2′H-5,5′-bitetrazol-2-yl}acetic acid

Ethyl{2′-[4-(2-bromo-5-fluorophenoxy)butyl]-2H,2′H-5,5′-bitetrazol-2-yl}acetate (major isomer: Rf: 0.5 with 10% EtOAc/CHCl₃) from step 5 was taken up in MeOH:THF (1:2) and treated with 1 N NaOH. After 15 min, the reaction was poured into aqueous 1 N HCl, extracted with EtOAc and washed with brine. The organic layer was dried (Na₂SO₄) and filtered.

Solvents were removed under diminished pressure and the crude material was triturated with Et₂O/hexanes to afford the title compound as a solid (regioisomeric ratio 23:1). ¹H NMR (400 MHz, DMSO-d₆): δ 13.88 (br s, 1H), 7.60 (dd, 1H), 7.09 (dd, 1H), 6.78 (td, 1H), 5.90 (s, 2H), 4.97 (t, 2H), 4.15 (t, 2H), 2.27-2.16 (m, 2H), 1.89-1.79 (m, 2H). MS: m/z=441.0, 439.0 (M-H).

The additional Examples listed in the Table below were prepared following the methods described for Examples 1-32.

| | Characterisation by |
|---|---|
| Examples 33- | Mass Spectrometry |
| 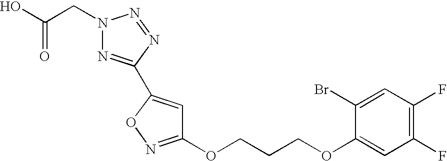 | MS: m/z 461.7, 459.8 (MH+) |
| 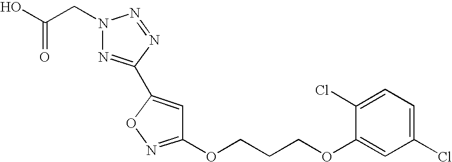 | MS: m/z 415.8, 413.9 (MH+) |
| 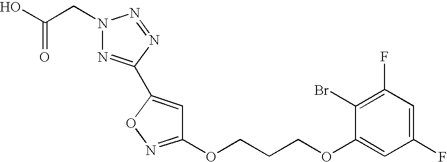 | MS: m/z = 483.9, 481.9 (M + Na) |
| 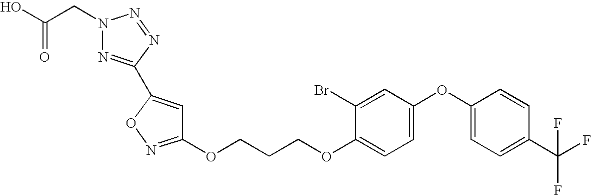 | MS: m/z = 608.1, 606.0 (M + Na) |
| 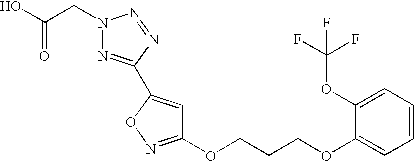 | MS: m/z = 452.2 (M + Na) |
| 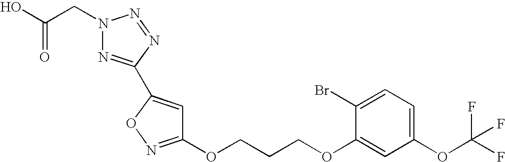 | MS: m/z = 532.1, 530.1 (M + Na) |
| 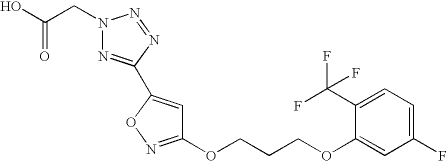 | MS: m/z = 454.2 (M + Na) |
| 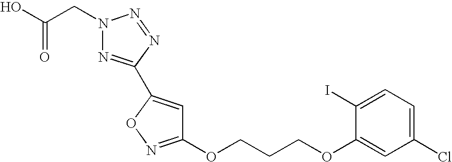 | MS: m/z 506.0, 503.9 (M − H) |
| 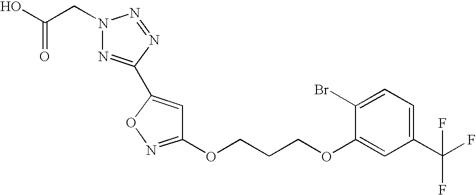 | MS: m/z 492.0, 490.0 (M − H) |
| 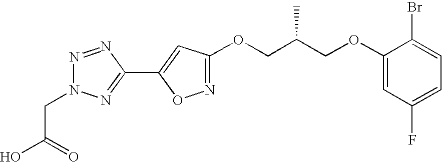 | MS: m/z 456.0, 453.9 (M − H) |
| 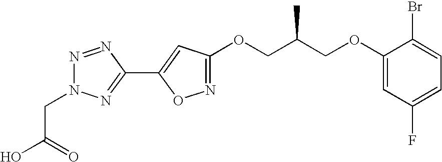 | MS: m/z 455.9, 454.0 (M − H) |
| 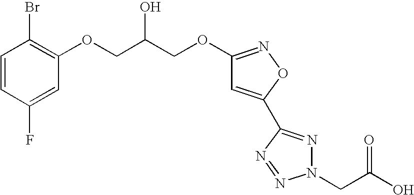 | MS: m/z = 460.1, 458.1 (MH+) |
| 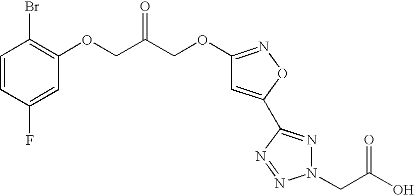 | MS: m/z = 458.0, 456.1 (MH+) |
| 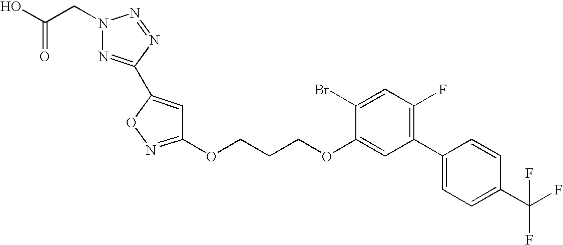 | MS: m/z 585.9, 583.8 (M − H) |
| 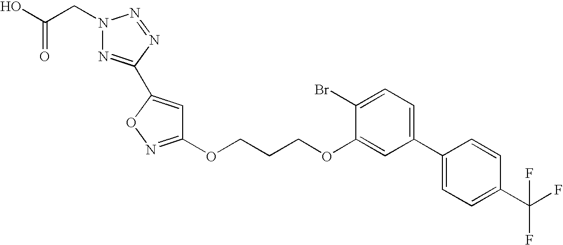 | MS: m/z 567.9, 566.0 (M − H) |
| 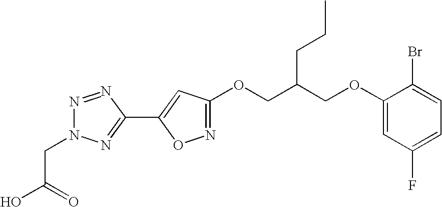 | MS: m/z = 484.0, 481.9 (M − H) |
| 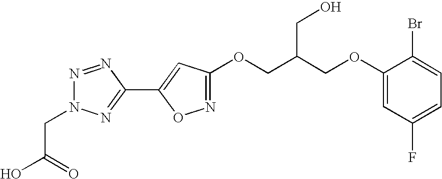 | MS: m/z = 471.9, 470.0 (M − H) |
| 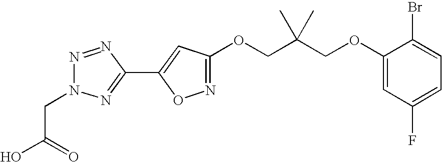 | MS: m/z = 469.9, 468.0 (M − H) |
| 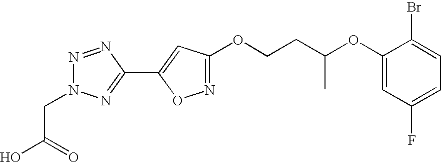 | MS: m/z = 457.8, 455.9 (MH+) |
| 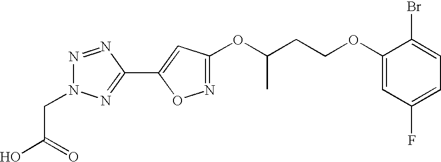 | MS: m/z = 458.0, 455.9 (MH+) |
| 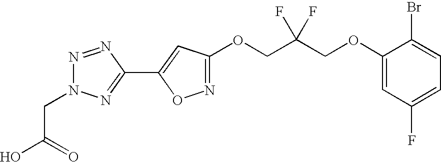 | MS: m/z = 479.8, 477.8 (MH+) |
| 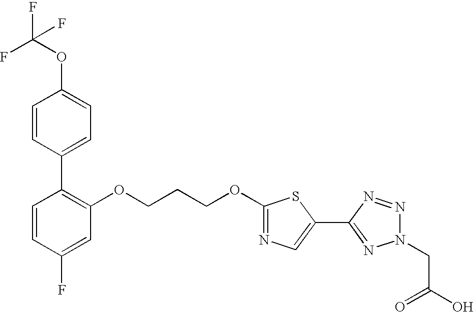 | MS: m/z = 538.0 (M − H) |
| 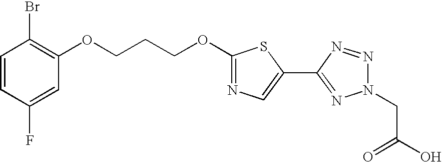 | MS: m/z = 457.8, 455.9 (M − H) |
| 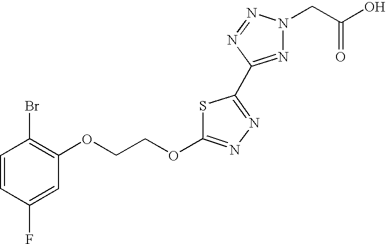 | MS: m/z = 444.8, 443.2 (M − H) |
| 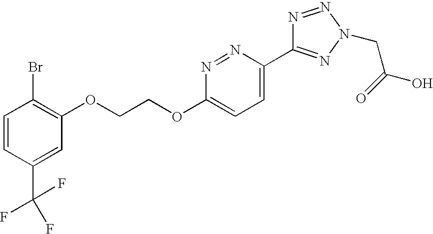 | MS: m/z = 488.9, 486.9 (M − H) |
| 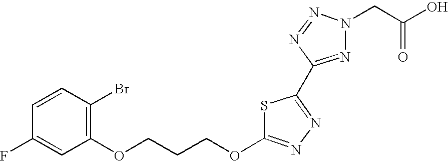 | MS: m/z = 460.9, 458.9 (MH+) |
| 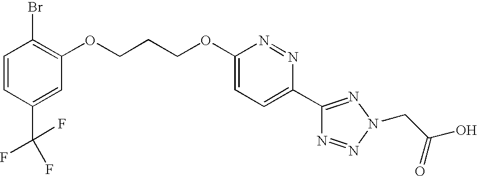 | MS: m/z = 504.9, 502.8 (MH+) |
| 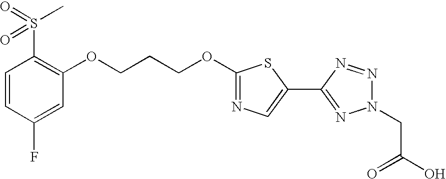 | MS: m/z = 458.1 (MH+) |
| 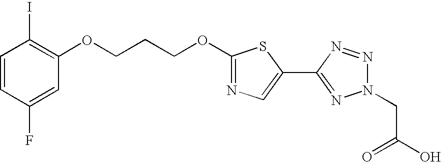 | MS: m/z = 506.1 (MH+) |
| 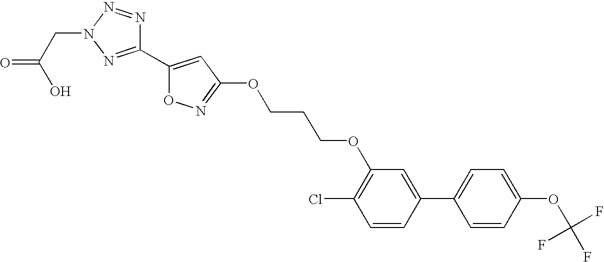 | MS: m/z = 540.4, 542.5 (MH+) |
| 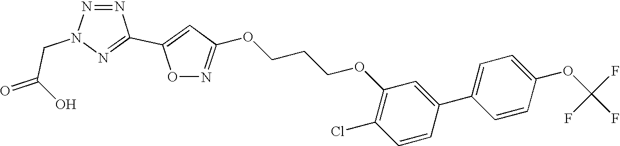 | MS: m/z = 526.4, 528.4 (MH+) |
| 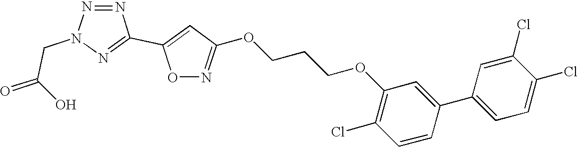 | MS: m/z = 524.3, 526.3 (MH+) |
| 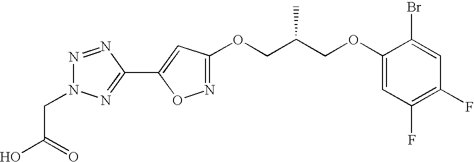 | MS: m/z = 476.0, 473.9 (MH+) |
| 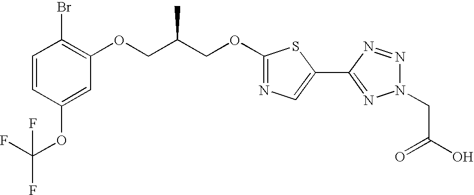 | MS: m/z = 539.9, 537.9 (MH+) |

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 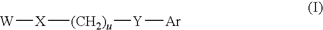
or a pharmaceutically acceptable salt thereof; wherein any methylene (CH₂) carbon atom in (CH₂)ᵤ is optionally substituted with one to two R⁵ substituents independently selected from fluorine, hydroxy, oxo, hydroxymethyl, and C₁₋₄ alkyl; or two R⁵ substituents, when on the same (CH₂) carbon atom, are taken together with the carbon atom to which they are attached to form a C₃₋₆ cycloalkyl group; or any two methylene (CH₂) carbon atoms are taken together to form a saturated or monounsaturated five- or six-membered cycloalkyl group;
X and Y are each independently a bond, —O—, —S—, —S(O)—, —S(O)₂—, —NR⁶—, 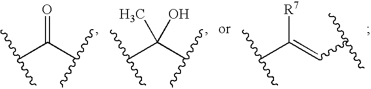
W is heteroaryl selected from the group consisting of: 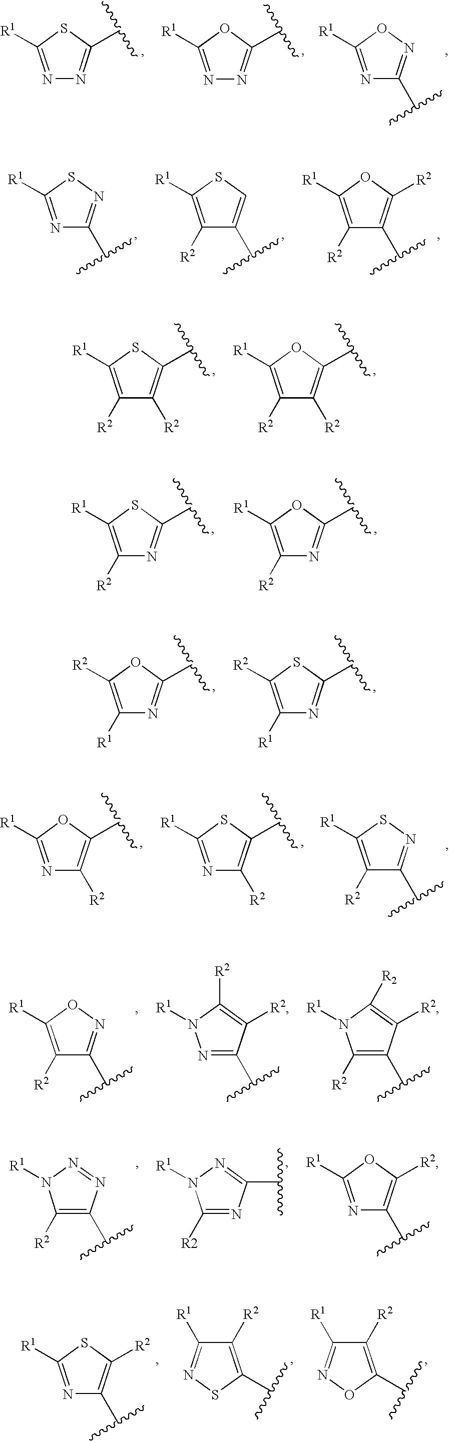 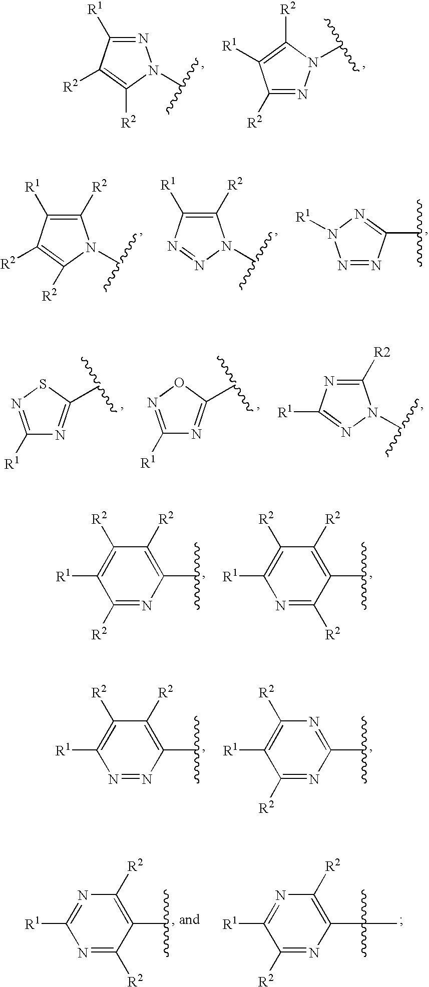
R¹ is heteroaryl selected from the group consisting of: 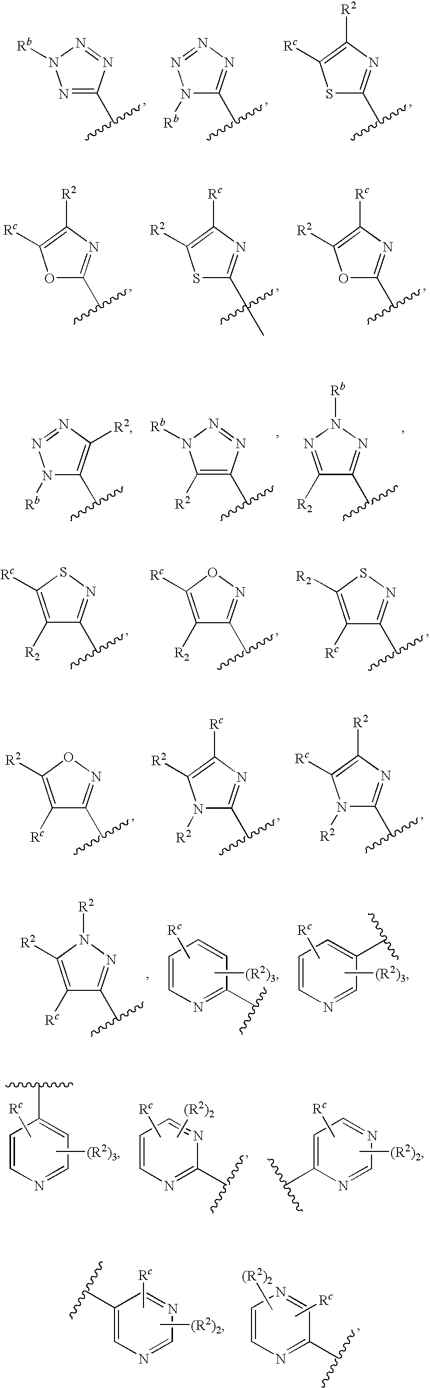 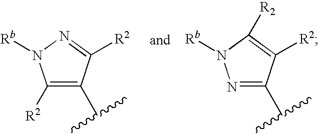
wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
and wherein said R¹ heteroaryl ring is optionally substituted with one substituent independently selected from the group consisting of cyano, halogen, C₁₋₄ alkyl, C₁₋₄ alkoxy, C₁₋₄ alkylthio, C₁₋₄ alkylsulfonyl, and trifluoromethyl;
each R² is independently selected from the group consisting of:
hydrogen,
halogen,
hydroxy,
cyano,
amino,
nitro,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl,
carboxy,
C₁₋₄ alkyloxycarbonyl, and
C₁₋₄ alkylcarbonyl;
Ar is phenyl or naphthyl optionally substituted with one to five R³ substituents;
each R³ is independently selected from the group consisting of:
C₁₋₆ alkyl,
C₂₋₆ alkenyl,
(CH₂)ₙ-phenyl,
(CH₂)ₙ-naphthyl,
(CH₂)ₙ-heteroaryl,
(CH₂)ₙ-heterocyclyl,
(CH₂)ₙC₃₋₇ cycloalkyl,
halogen,
nitro,
(CH₂)ₙOR⁴,
(CH₂)ₙN(R⁴)₂,
(CH₂)ₙC≡N,
(CH₂)ₙCO₂R⁴,
(CH₂)ₙNR⁴SO₂R⁴
(CH₂)ₙSO₂N(R⁴)₂,
(CH₂)ₙS(O)₀₋₂R⁴,
(CH₂)ₙNR⁴C(O)N(R⁴)₂,
(CH₂)ₙC(O)N(R⁴)₂,
(CH₂)ₙNR⁴C(O)R⁴,
(CH₂)ₙNR⁴CO₂R⁴,
(CH₂)ₙC(O)R⁴,
O(CH₂)ₙC(O)N(R⁴)₂,
(CH₂)ₛ—Z—(CH₂)ₜ-phenyl,
(CH₂)ₛ—Z—(CH₂)ₜ-naphthyl,
(CH₂)ₛ—Z—(CH₂)ₜ-heteroaryl,
(CH₂)ₛ—Z—(CH₂)ₜ-heterocyclyl,
(CH₂)ₛ—Z—(CH₂)ₜ—C₃₋₇ cycloalkyl,
(CH₂)ₛ—Z—(CH₂)ₜ—OR⁴,
(CH₂)ₛ—Z—(CH₂)ₜ—N(R⁴)₂,
(CH₂)ₛ—Z—(CH₂)ₜ—NR⁴SO₂R⁴,
(CH₂)ₛ—Z—(CH₂)ₜ—C≡N,
(CH₂)ₛ—Z—(CH₂)ₜ—CO₂R⁴,
(CH₂)ₛ—Z—(CH₂)ₜ—SO₂N(R⁴)₂,
(CH₂)ₛ—Z—(CH₂)ₜ—S(O)₀₋₂R⁴,
(CH₂)ₛ—Z—(CH₂)ₜ—NR⁴C(O)N(R⁴)₂,
(CH₂)ₛ—Z—(CH₂)ₜ—C(O)N(R⁴)₂,
(CH₂)ₛ—Z—(CH₂)ₜ—NR⁴C(O)R⁴,
(CH₂)ₛ—Z—(CH₂)ₜ—NR⁴CO₂R⁴,
(CH₂)ₛ—Z—(CH₂)ₜ—C(O)R⁴,
CF₃,
CH₂CF₃,
OCF₃, and
OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy optionally substituted with one to five fluorines; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
each R⁴ is independently selected from the group consisting of
hydrogen,
C₁₋₆ alkyl,
(CH₂)ₙ-phenyl,
(CH₂)ₙ-heteroaryl,
(CH₂)ₙ-naphthyl, and
(CH₂)ₙC₃₋₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
each R⁶ and R⁷ are independently hydrogen or C₁₋₃ alkyl, wherein alkyl is optionally substituted with one to five fluorines;
u is an integer from 1 to 4;
r is an integer from 1 to 3;
m is an integer from 0 to 3;
each p is independently an integer from 1 to 3;
each n is independently an integer from 0 to 2;
each s is independently an integer from 1 to 3; and
each t is independently an integer from 1 to 3.

2. The compound of claim 1 wherein X and Y are both O.

3. The compound of claim 1 wherein u is 3.

4. The compound of claim 3 wherein X and Y are both O.

5. The compound of claim 3 wherein X is S and Y is O.

6. The compound of claim 1 wherein Ar is phenyl substituted with one to three R³ substituents.

7. The compound of claim 1 wherein W is heteroaryl selected from the group consisting of:

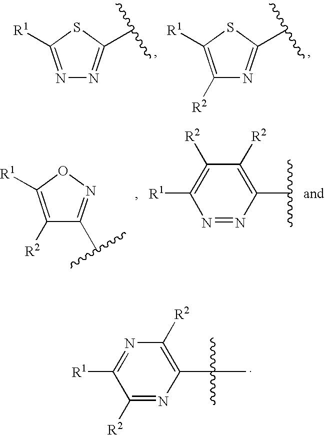

8. The compound of claim 7 wherein R² is hydrogen.

9. The compound of claim 7 wherein W is

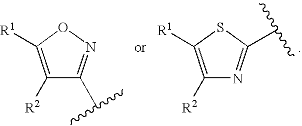

10. The compound of claim 9 wherein R² is hydrogen.

11. The compound of claim 9 wherein W is

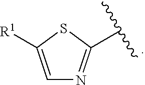

12. The compound of claim 1 wherein R¹ is heteroaryl selected from the group consisting of: 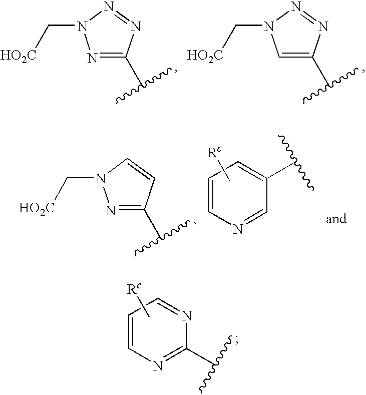
wherein R^{c} is —CO₂H, —CO₂C₁₋₃ alkyl, —CH₂CO₂H, or —CH₂CO₂C₁₋₃ alkyl.

13. The compound of claim 12 wherein R¹ is

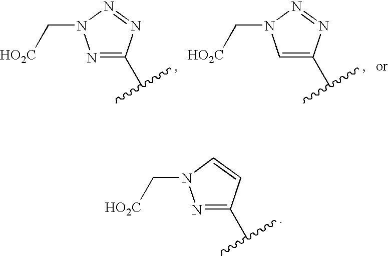

14. The compound of claim 1 wherein W is heteroaryl selected from the group consisting of: 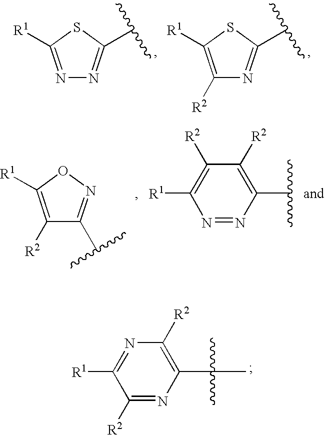
and R¹ is heteroaryl selected from the group consisting of: 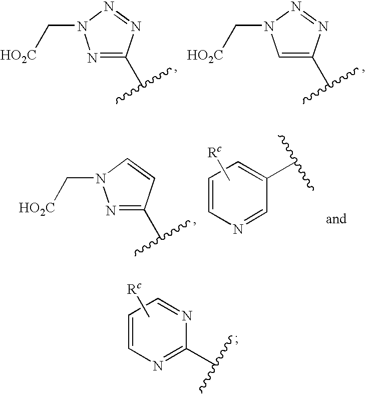
wherein R^{c} is —CO₂H, —CO₂C₁₋₃ alkyl, —CH₂CO₂H, or —CH₂CO₂C₁₋₃ alkyl.

15. The compound of claim 14 wherein W is 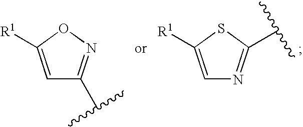
and R¹ is

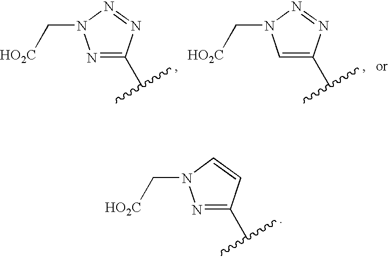

16. The compound of claim 15 wherein W is

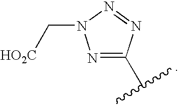

17. A compound which is selected from the group consisting of: 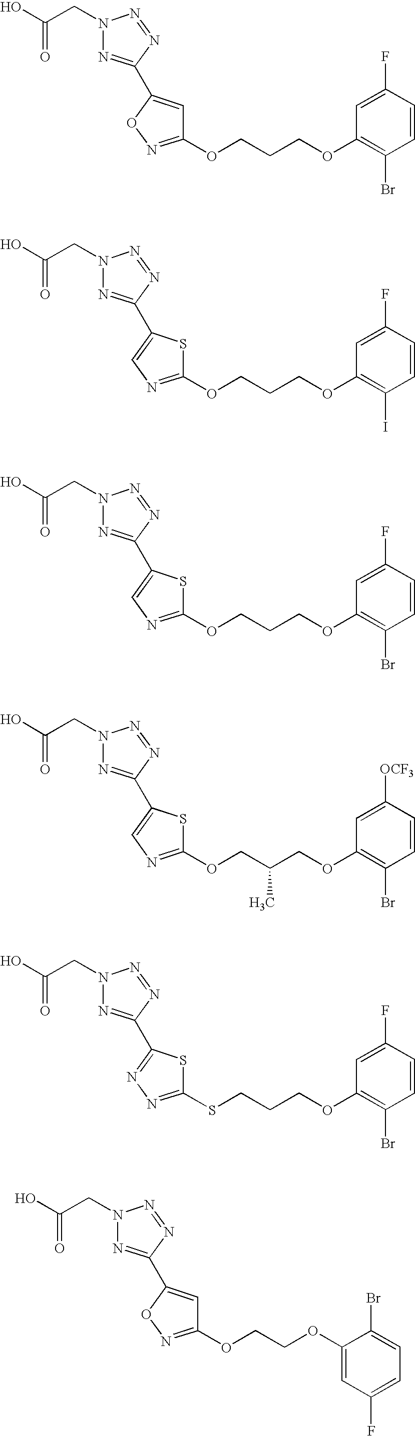 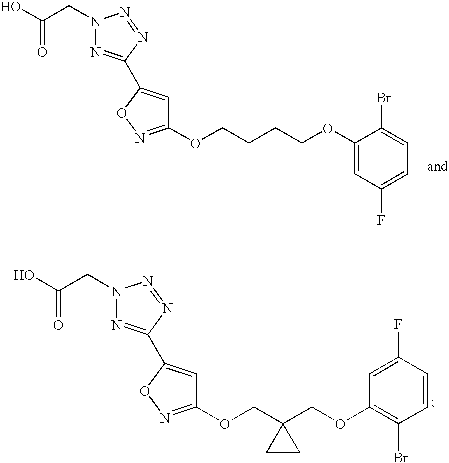
or a pharmaceutically acceptable salt thereof.

18. A pharmaceutical composition comprising a compound in accordance with claim 1 in combination with a pharmaceutically acceptable carrier.

19-23. (canceled)

24. A method for treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

25. The method of claim 24 wherein said lipid disorder is selected from the group consisting of dyslipidemia, hyperlipidemia, hypertriglyceridemia, atherosclerosis, hypercholesterolemia, low HDL, and high LDL.
